(19) United States
(12) Patent Application Publication
(10) Pub. No.: US 2009/0318561 A1
(43) Pub. Date: Dec. 24, 2009

(54) COLCHICINE PRODUCTS, METHOD OF MANUFACTURE, AND METHODS OF USE

(75) Inventors: Matthew W. Davis, Erwinna, PA (US); Jie Du, Lansdale, PA (US); Kurt R. Nielsen, Chadds Ford, PA (US); Richard H. Roberts, Lakewood, NJ (US)

Correspondence Address: CANTOR COLBURN, LLP, 20 Church Street, 22nd Floor, Hartford, CT, 06103, US

(73) Assignee: MUTUAL PHARMACEUTICAL COMPANY, INC., Philadelphia, PA (US)

(21) Appl. No.: 12/489,750

(22) Filed: Jun. 23, 2009

Related U.S. Application Data
(60) Provisional application No. 61/074,818, filed on Jun. 23, 2008.

(51) Int. Cl. A61K 31/165; A61P 19/06; G06Q 50/00

(52) U.S. Cl. 514/625; 705/2

(57) ABSTRACT

Disclosed herein is a method of using colchicine. In one embodiment, the method comprises administering to a patient colchicine and a substrate of cytochrome P450 1A2 and monitoring the patient during administration of colchicine and the substrate for an adverse event. Also disclosed are articles of manufacture comprising a container containing a dosage form of colchicine and a method of manufacturing a colchicine product.

### CROSS REFERENCE TO RELATED APPLICATION

This application claims the benefit of U.S. Provisional Application Ser. No. 61/074,818 filed Jun. 23, 2008, hereby incorporated by reference in its entirety.

### BACKGROUND

This application relates to colchicine products for therapeutic purposes, and in particular to improved methods of use of colchicine.

Colchicine is an alkaloid originally prepared from the dried corms and seeds of Colchicum autumnale, the autumn crocus or meadow saffron. The chemical name for colchicine is (S)N-(5, 6, 7, 9-tetrahydro-1,2,3,10-tetramethoxy-9-oxobenzo[alpha]heptaien-7-yl) acetamide. It is a pale yellow powder soluble in water in 1:25 dilution.

Colchicine is used for treatment and relief of pain and other symptoms of attacks of acute gouty arthritis (also called acute gouty flares or plainly, acute gout), which may include swelling, redness and warmth. It is also recommended for regular use between attacks as a prophylactic measure for chronic gout.

Colchicine is a microtubule-disrupting agent used in the treatment of gout, particularly in the treatment of acute gouty arthritis. Colchicine impairs the motility of granulocytes and can prevent the inflammatory phenomena that initiate an attack of gout. Colchicine also inhibits mitosis, thus affecting cells with high turnover such as those in the gastrointestinal tract and bone marrow; therefore, the primary side effects include gastrointestinal upset such as diarrhea and nausea. Colchicine is typically administered in 1- to 1.2-mg doses, with follow-up doses of 0.5 to 0.6 mg twice daily. The beneficial effects of colchicine in the treatment of acute gouty flares has traditionally taken up to 48 hours to manifest; therefore, multi-dose therapy is likely during the treatment of gout.

One of the most important groups of Phase I metabolic enzymes are the cytochrome p450 monooxygenase system enzymes. The cytochrome p450 enzymes are a highly diverse superfamily of enzymes. NADPH is required as a coenzyme and oxygen is used as a substrate. Each enzyme is termed an isoform or isozyme since each derives from a different gene.

Many members of the cytochrome p450 family are known to metabolize active agents in humans. Active agent interactions associated with metabolism by cytochrome p450 isoforms generally result from enzyme inhibition or enzyme induction. Enzyme inhibition often involves competition between two active agents for the substrate-binding site of the enzyme, although other mechanisms for inhibition exist. Enzyme induction occurs when an active agent activates an enzyme or stimulates the synthesis of more enzyme protein, enhancing the enzyme's metabolizing capacity.

Cytochrome p450 isozymes identified as important in active agent metabolism are CYP1A2, CYP2A6, CYP2B6, CYP2C8, CYP2C9, CYP2C19, CYP2D6, CYP2E1, and CYP3A4. Examples of cytochrome p450 enzymes known to be involved in active agent interactions are the CYP3A subfamily, which is involved in many clinically significant active agent interactions, including those involving non-sedating antihistamines and cisapride, and CYP2D6, which is responsible for the metabolism of many psychotherapeutic agents, such as thioridazine. CYP1A2 and CYP2E1 enzyme are involved in active agent interactions involving theophylline. CYP2C9, CYP1A2, and CYP2C19 are involved in active agent interactions involving warfarin. Phenyloin and fosphenyloin are metabolized by CYP2C9, CYP2C19, and CYP3A4.

Additionally, several cytochrome p450 isozymes are known to be genetically polymorphic, leading to altered substrate metabolizing ability in some individuals. Allelic variants of CYP2D6 are the best characterized, with many resulting in an enzyme with reduced, or no, catalytic activity. Gene duplication also occurs. As a result, four phenotypic subpopulations of metabolizers of CYP2D6 substrates exist: poor (PM), intermediate (IM), extensive (EM), and ultrarapid (UM). The genetic polymorphisms vary depending on the population in question. For example, Caucasian populations contain a large percentage of individuals who are poor metabolizers, due to a deficiency in CYP2D6—perhaps 5-10% of the population, while only 1-2% of Asians are PMs. CYP2C9, which catalyzes the metabolism of a number of commonly used active agents, including that of warfarin and phenyloin, is also polymorphic. The two most common CYP2C9 allelic variants have reduced activity (5-12%) compared to the wild-type enzyme. Genetic polymorphism also occurs in CYP2C19, for which at least 8 allelic variants have been identified that result in catalytically inactive protein. About 3% of Caucasians are poor metabolizers of active agents metabolized by CYP2C19, while 13-23% of Asians are poor metabolizers of active agents metabolized by CYP2C19. Allelic variants of CYP2A6 and CYP2B6 have also been identified as affecting enzyme activity. At least one inactive CYP2A6 variant occurs in Caucasians at a frequency of 1-3%, resulting in a PM phenotype. A whole gene deletion has been identified in a Japanese population, with an allelic frequency of 21%; homozygotes in this mutation show a PM phenotype. For CYP2B6, about 3-4% of Caucasians have a polymorphism producing a PM phenotype.

Tateishi et al. (Biochem. Pharmacol. (1997) 53:111-116) studied the biotransformation of colchicine in human liver microsomes in order to identify particular human cytochrome P450 isozymes responsible for the formation of its demethylated metabolites. Formation of 3-demethylchochicine and 2-demethylcolchicine was correlated with CYP3A4 activity, but not with activity of CYP2A6, CYP2C9, CYP2C19, CYP2D6, or CYP2E1. Metabolism of colchicine by CYP3A4 was confirmed by using antibodies against CYP3A4 and chemical inhibition of CYP3A4.

Studies on the effect of colchicine on expression of selected cytochrome P450 isozymes in primary cultures of human hepatocytes have also been published. Dvorak et al. (Acta Univ. Palacki. Olomuc., Fac. Med. (2000) 143:47-50) provided preliminary data on the effect of colchicine and several of its derivatives on protein levels of CYP1A2, CYP2A6, CYP2C9/19, CYP2E1, and CYP3A4 by immunoblotting. Colchicine caused an increase in CYP2E1 protein levels and appeared to decrease protein levels of CYP1A2, CYP2C9/19, and CYP3A4, with 10 μM colchicine causing a greater reduction in each isozyme than 1 μM colchicine. The colchicine metabolite 3-demethylchochicine caused a decrease in protein for CYP1A2, CYP2C9/19, CYP2E1, and CYP3A4. The levels of CYP2A6 were unaffected by colchicine or any of the tested metabolites. In a more complete report on expression of CYP1A2, CYP2A6, CYP2C9, CYP2C19, CYP2E1, and CYP3A4 in primary cultures of human hepatocytes, Dvorak et al. (Toxicology in Vitro (2002) 16:219-227) concluded that CYP1A2 protein content in 1 μM colchicine treated cells was not different from that in control cells, while the inducer TCDD increased the level of CYP1A2 protein by an average of three-fold. The levels of CYP2A6 protein were also unaffected by colchicine. The enzyme activities of CYP3A4 and CYP2C9 were significantly decreased by colchicine, whereas activity of CYP2E1 was not affected. Northern blots showed that colchicine suppressed CYP2C9 mRNA levels by about 20% and did not alter CYP3A4 mRNA levels as compared to control cells. A subsequent study by Dvorak et al. (Mol. Pharmacol. (2003) 64:160-169) showed that colchicine decreased both basal and rifampicin-inducible and phenobarbital-inducible expression of CYP2B6, CYP2C8/9, and CYP3A4.

Active agent interactions present a health risk to patients and a medical challenge for all medical care workers. Various studies of adverse reactions from exposure to active agents have found that 6.5-23% of the adverse reactions result from active agent interactions. Unfortunately, each year a number of deaths occur as the direct result of patients taking a new prescription pharmaceutical product in combination with their existing medication regimen. By understanding the unique functions and characteristics of Phase I and Phase II metabolic enzymes, such as the cytochrome p450 enzyme superfamily, medical care workers such as physicians and pharmacists may better avoid or safely manage active agent interactions and may better anticipate or explain an individual's response to a particular therapeutic regimen.

There accordingly remains a need in the art for improved methods for the administration and use of colchicine, in particular methods that take into account the effects of colchicine on metabolism by cytochrome P450 isozymes.

### SUMMARY

Disclosed herein are methods of using colchicine. Colchicine can be used in prevention or treatment of various diseases or conditions, including, for example, attacks of acute gouty arthritis and pain and other symptoms in attacks of acute gouty arthritis, chronic gout (prophylaxis of gouty arthritis), a cystic disease, for example polycystic kidney disease or cystic fibrosis, a lentiviral infection, demyelinating diseases of central or peripheral origin, multiple sclerosis, cancer, an inflammatory disorder such as rheumatoid arthritis, glaucoma, Dupuytren's contracture, idiopathic pulmonary fibrosis, primary amyloidosis, recurrent pericarditis, acute pericarditis, asthma, postpericardiotomy syndrome, proliferative vitreoretinopathy, Behçet's disease, Familial Mediterranean fever, idiopathic thrombocytopenic purpura, primary biliary cirrhosis, and pyoderma gangrenosum, or in enhancing bone formation or bone mineral density.

In an embodiment, the method comprises administering colchicine and a substance that is a substrate of cytochrome P450 1A2, 2A6, 2C19, 2D6, or 2E1 to a patient; and monitoring the patient during administration of colchicine and the substance.

In an embodiment, the method comprises administering colchicine to a patient in need thereof; determining that the patient is taking a substance that is a substrate of cytochrome P450 1A2, 2A6, 2C19, 2D6, or 2E1; and adjusting administration of colchicine or the substance to the patient to avoid an adverse reaction.

In an embodiment, the method comprises determining that a patient in need colchicine therapy is taking a substance that is a substrate of cytochrome P450 1A2, 2A6, 2C19, 2D6, or 2E1, and adjusting administration to the patient of colchicine or the substance to avoid an adverse event associated with suppression of metabolism of the substance by colchicine.

In an embodiment, the method comprises administering colchicine and a substrate of cytochrome P450 1A2, 2A6, 2C19, 2D6, or 2E1 to a patient; and altering dosing of the substrate or colchicine for the patient if substrate plasma concentration of the patient increases during coadministration with colchicine.

In an embodiment, the method comprises administering colchicine and a substrate of cytochrome P450 1A2, 2A6, 2C19, 2D6, or 2E1 to a patient; determining that the patient experiences a substrate-associated toxicity during coadministration with colchicine; and altering dosing of the substrate or colchicine such that the substrate-associated toxicity is reduced.

In an embodiment, the method comprises administering colchicine to a patient in need of colchicine therapy; determining that a substance that is a substrate of cytochrome P450 1A2, 2A6, 2C19, 2D6, or 2E1 is administered to the patient; and monitoring the patient during administration of colchicine and the substance.

In an embodiment, the method comprises determining for a patient to whom colchicine is going to be administered or is being administered whether a substance that is currently being or will be administered to the patient is a substrate of a cytochrome P450 1A2 (CYP1A2); and determining risk for the patient of an adverse event resulting from reduced metabolism of the substance by CYP1A2 during coadministration of colchicine and the substance.

In an embodiment, the method comprises determining a dosing regimen for a substrate of cytochrome P450 1A2 to be administered to a patient in need thereof; determining that colchicine is administered to the patient, and altering the determined dosing regimen of the substrate during coadministration of colchicine to prevent a substrate-associated toxicity.

In an embodiment, the method comprises informing a user that colchicine is metabolized by cytochrome P450 2A6, 2B6, 2C8, 2C9 or 2C19; inhibited cytochrome P450 2A6 or 2C8 enzyme activity in an in vitro inhibition study; activated CYP3A4 enzyme activity in an in vitro inhibition study; suppressed enzyme activity of cytochrome P450 1A2, 2A6, 2C19, 2D6, or 2E1 in an in vitro induction study; or suppressed mRNA expression of cytochrome P450 1A2 in an in vitro induction study.

In an embodiment, the method comprises obtaining colchicine from a container associated with published material providing information that colchicine is metabolized by cytochrome P450 2A6, 2B6, 2C8, 2C9 or 2C19; inhibited cytochrome P450 2A6 or 2C8 enzyme activity in an in vitro inhibition study; activated CYP3A4 enzyme activity in an in vitro inhibition study; suppressed enzyme activity of cytochrome P450 1A2, 2A6, 2C19, 2D6, or 2E1 in an in vitro induction study; or suppressed mRNA expression of cytochrome P450 1A2 in an in vitro induction study.

Also disclosed herein are methods of manufacturing a colchicine product.

In one embodiment, the method comprises packaging a colchicine dosage form with published material providing information that colchicine is metabolized by cytochrome P450 2A6, 2B6, 2C8, 2C9 or 2C19; inhibited cytochrome P450 2A6 or 2C8 enzyme activity in an in vitro inhibition study; activated CYP3A4 enzyme activity in an in vitro inhibition study; suppressed enzyme activity of cytochrome P450 1A2, 2A6, 2C19, 2D6, or 2E1 in an in vitro induction study; or suppressed mRNA expression of cytochrome P450 1A2 in an in vitro induction study.

Also disclosed herein are articles of manufacture comprising a container containing a dosage form of colchicine.

In one embodiment, the container is associated with published material informing that colchicine is metabolized by cytochrome P450 2A6, 2B6, 2C8, 2C9 or 2C19; inhibited cytochrome P450 2A6 or 2C8 enzyme activity in an in vitro inhibition study; activated CYP3A4 enzyme activity in an in vitro inhibition study; suppressed enzyme activity of cytochrome P450 1A2, 2A6, 2C19, 2D6, or 2E1 in an in vitro induction study; or suppressed mRNA expression of cytochrome P450 1A2 in an in vitro induction study

These and other embodiments, advantages and features of the present invention become clear when detailed description and examples are provided in subsequent sections.

### DETAILED DESCRIPTION

Disclosed herein are methods of using colchicine and colchicine products. The inventors have determined certain effects of colchicine on the activity of various cytochrome P450 isozymes and identified risks associated with administration of colchicine with another substance resulting from these effects of colchicine on the activity of the cytochrome P450 isozymes. With the knowledge of the particular information, a medical care worker can better avoid or safely manage an active agent interaction in a patient between colchicine and the substance, and its resultant effects on efficacy or safety of colchicine or the substance. Specifically, knowledge of the particular information permits the administration of colchicine or the substance to be optimized for the patient by a medical care worker to provide safe use of colchicine or the substance, while oftentimes reducing or minimizing side effects or adverse events resulting from the effects. Knowledge of the particular information permits a medical care worker to use colchicine to treat a patient that is taking another substance more effectively and with fewer risks by allowing proper dosing, dispensing, and administration of colchicine or the substance to the patient by the patient's medical care worker to avoid, or reduce risk of occurrence of a sub-therapeutic effect, a side effect, an adverse reaction, or an active agent interaction between colchicine and the substance and alerts the patient and the patient's medical care worker to the need to monitor the patient for symptoms of a sub-therapeutic effect, a side effect, an adverse reaction, or an active agent interaction between colchicine and the substance.

Enzymes involved in Phase I and Phase II active agent metabolism, such as the cytochrome p450 isozymes, respond to the constantly changing types and amounts of substrates they encounter. For example, changes in active agent metabolism due to competition for the same cytochrome P450 isoform can change the clinical effectiveness or safety of an active agent by altering the plasma concentration of the active agent or its metabolite(s). Similarly, inhibition or induction of the cytochrome P450 isoform that metabolizes a particular active agent can change the clinical effectiveness or safety of that active agent. For the case in which the active agent is a narrow therapeutic index active agent, such as warfarin or phenyloin, too little of the active agent in the blood stream can lead to insufficient therapeutic activity, while a too large dose of the active agent can lead to excessive therapeutic activity or toxicity, either of which can be detrimental to the patient. For example, since colchicine down-regulates CYP1A2 mRNA expression and enzyme activity in primary cultures of human hepatocytes, the administration of colchicine with a substance that is a substrate of CYP1A2 can decrease the metabolism by CYP1A2 of that substrate.

Colchicine therapy can be considered optimal when effective plasma levels are reached when required. In addition, peak plasma values (Cₘₐₓ) should be as low as possible so as to reduce the incidence and severity of possible side effects.

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

An “active agent” means a compound (including for example, colchicine), element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates), and co-crystals of the free compound or salt, crystalline forms, non-crystalline forms, and any polymorphs of the compound are contemplated herein. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, free compound, and salts) of colchicine or other active agent may be employed either alone or in combination.

“Active agent interaction” refers to a change in the metabolism or the pharmacology of an active agent in a patient that can occur with co-administration of a second active agent. A “potential active agent interaction” refers to an active agent interaction between two active agents that is theoretically possible based on knowledge that one of the active agents is metabolized by a given cytochrome p450 isozyme and that the second of the active agents is a substrate, inhibitor, or inducer of that cytochrome p450 isozyme.

“Administering colchicine with a substance”, “administering colchicine and a substance”, or “co-administering colchicine and a substance” means colchicine and the substance are administered simultaneously in a single dosage form, administered concomitantly in separate dosage forms, or administered in separate dosage forms separated by some amount of time that is within the time in which both colchicine and the substance are within the blood stream of a patient. The colchicine and the substance need not be prescribed for a patient by the same medical care worker. The substance need not require a prescription. Administration of colchicine or the substance can occur via any appropriate route, for example, oral tablets, oral capsules, oral liquids, inhalation, injection, suppositories, or topical contact.

“Adverse event” means any untoward medical occurrence in a patient administered an active agent and which does not necessarily have to have a causal relationship with this treatment. An adverse event (AE) can therefore be any unfavorable and unintended sign (including an abnormal laboratory finding, for example), symptom, or disease temporally associated with the use of the active agent, whether or not considered related to the active agent.

“Adverse reaction” means a response to an active agent which is noxious and unintended and which occurs at doses normally used in humans for prophylaxis, diagnosis, or therapy of disease or for modification of physiological function. The unintended response can be an unexpected diminished or enhanced pharmacologic activity or toxicity of the active agent, e.g., a colchicine-associated toxicity. An adverse reaction also includes any undesirable or unexpected event requiring discontinuation of the active agent, modification of the dose, prolonged hospitalization, or the administration of supportive treatment.

“Affects” include an increase or decrease in degree, level, or intensity; a change in time of onset or duration; a change in type, kind, or effect, or a combination comprising at least one of the foregoing.

As used herein, “allelic variant” means one of the alternative forms at a genetic locus on a single chromosome. For loci in most of the human genome, a human has two chromosomes, which may carry the same or two different allelic variants.

“Adjusting administration of an active agent”, “altering administration of an active agent”, or “altering” or “adjusting” dosing of an active agent are all equivalent and mean making no change in the dose or dosing regimen of the active agent; tapering off, reducing or increasing the dose or the interval between doses of the active agent, ceasing to administer the active agent to the patient, or substituting a different active agent for the active agent.

“Dosing regimen” means the dose of an active agent taken at a first time by a patient and the interval (time or symptomatic) at which any subsequent doses of the active agent are taken by the patient. The additional doses of the active agent can be different from the dose taken at the first time.

A “dose” means the measured quantity of an active agent to be taken at one time by a patient.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

A “dosage form” means a unit of administration of an active agent. Examples of dosage forms include tablets, capsules, injections, suspensions, liquids, emulsions, creams, ointments, suppositories, inhalable forms, transdermal forms, and the like.

The term “effective amount” or “therapeutically effective amount” means an amount effective, when administered to a patient, to provide any therapeutic benefit. A therapeutic benefit may be an amelioration of symptoms, e.g., an amount effective to decrease the symptoms of acute gouty arthritis, for example pain associated with an attack of acute gouty arthritis. The amount that is “effective” will vary from subject to subject, depending on the age and general condition of the individual, the particular active agent, and the like. Thus, it is not always possible to specify an exact “effective amount.” However, an appropriate “effective” amount in any individual case may be determined by one of ordinary skill in the art using routine experimentation. In certain circumstances a patient may not present symptoms of a condition for which the patient is being treated. A therapeutically effective amount of an active agent may also be an amount sufficient to provide a significant positive effect on any indicium of a disease, disorder, or condition, e.g. an amount sufficient to significantly reduce the severity of an attack of acute gouty arthritis. A significant effect on an indicium of a disease, disorder, or condition is statistically significant in a standard parametric test of statistical significance, for example Student's T-test, where p≦0.05. An “effective amount” or “therapeutically effective amount” of colchicine may also be an amount of about 10 mg per day or less, specifically about 8 mg per day or less, or of any dosage amount approved by a governmental authority such as the United States Food and Drug Administration (FDA), for use in treatment. For example, an effective amount can be up to 4.8 mg colchicine per incident of acute gout, or 0.5 or 0.6 mg colchicine twice daily for either prophylaxis of chronic gout or treatment of Behçet's disease or Familial Mediterranean fever. In some embodiments amounts of 8 mg colchicine per day, 1.0 or 1.2 mg colchicine per unit dosage form, or 0.5 or 0.6 mg colchicine or less per unit dosage form is an “effective amount” or “therapeutically effective amount” of colchicine.

“Efficacy” means the ability of an active agent administered to a patient to produce a therapeutic effect in the patient.

“Enhancing the safety profile” of an active agent means implementing actions or articles designed or intended to help reduce the incidence of adverse events associated with administration of the active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

“Informing” means referring to or providing published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

“Labeling” means all labels or other means of written, printed, graphic, electronic, verbal, or demonstrative communication that is upon a dosage form or packaging of a pharmaceutical product or that accompanies a dosage form in a pharmaceutical product.

A “medical care worker” means a worker in the health care field who may need or utilize information regarding an active agent, including a dosage form thereof, including information on safety, efficacy, dosing, administration, or pharmacokinetics. Examples of medical care workers include physicians, pharmacists, physician's assistants, nurses, aides, caretakers (which can include family members or guardians), emergency medical workers, and veterinarians.

As used herein, an enzyme “metabolizing” a substance means the substance is a substrate of the enzyme, i.e., the enzyme can chemically transform the substance.

A substance having a “narrow therapeutic index” (NTI) means a substance falling within any definition of narrow therapeutic index as promulgated by the U.S. Food and Drug Administration or any successor agency thereof. For example, a substance having a “narrow therapeutic index” can be a substance having a less than 2-fold difference in median lethal dose (LD50) and median effective dose (ED50) values or having a less than 2-fold difference in the minimum toxic concentration and minimum effective concentration in the blood; and for which safe and effective use of the substance requires careful titration and patient monitoring.

“Oral dosage form” includes a dosage form for oral administration.

A “patient” means a human or non-human animal in need of medical treatment. Medical treatment can include treatment of an existing condition, such as a disease or disorder, prophylactic or preventative treatment, or diagnostic treatment. In some embodiments the patient is a human patient.

A “pharmaceutical supplier” means a person (other than a medical care worker), business, charitable organization, governmental organization, or other entity involved in the transfer of active agent, including a dosage form thereof, between entities, for profit or not. Examples of pharmaceutical suppliers include pharmaceutical distributors, pharmaceutical wholesalers, pharmaceutical benefits managers, pharmacy chains, pharmacies (online or physical), hospitals, HMOs, supermarkets, the Veterans Administration, or foreign businesses or individuals importing active agent into the United States.

“Pharmacokinetic parameters” describe the in vivo characteristics of an active agent (or surrogate marker for the active agent) over time, such as plasma concentration (C), Cₘᵢₙ, Cₘₐₓ, Cₙ, C₂₄, Tₘₐₓ, and AUC. “Cₘₐₓ” is the measured concentration of the active agent in the plasma at the point of maximum concentration. “Cₘᵢₙ” is the measured concentration of the active agent in the plasma at the point of minimum concentration at steady state. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC_{0-INF} is the calculated area under the curve of plasma concentration versus time from time 0 to time infinity.

“Pharmaceutically acceptable salts” include derivatives of the active agent (e.g., colchicine), wherein the parent compound is modified by making acid or base addition salts thereof, and further refers to pharmaceutically acceptable solvates, including hydrates, and co-crystals of such compounds and such salts. All forms of such derivatives of colchicine are contemplated herein, including all crystalline, amorphous, and polymorph forms. Examples of pharmaceutically acceptable salts include, but are not limited to, mineral or organic acid addition salts; and the like, and combinations comprising one or more of the foregoing salts. The pharmaceutically acceptable salts include salts, for example, from inorganic or organic acids. For example, acid salts include those derived from inorganic acids such as hydrochloric, hydrobromic, sulfuric, sulfamic, phosphoric, nitric and the like. Pharmaceutically acceptable organic salts includes salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′ dibenzylethylenediamine salt, and the like; and amino acid salts such as arginate, asparginate, glutamate, and the like; and combinations comprising one or more of the foregoing salts. Specific colchicine salts include colchicine hydrochloride, colchicine dihydrochloride, and co-crystals, hydrates or solvates thereof.

“Phenotype” means an observable trait of an organism resulting from the interplay of environment and genetics. Examples include apparent rate of metabolism of substrates by a cytochrome p450 isozyme of an organism, such as the “poor metabolizer” (PM) or “ultrarapid metabolizer” (UM) phenotypes identified in humans for metabolism of substrates metabolized by CYP2D6.

A “product” or “pharmaceutical product” means a dosage form of an active agent plus published material, and optionally packaging.

“Providing” means giving, administering, selling, distributing, transferring (for profit or not), manufacturing, compounding, or dispensing.

“Published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, an audio recording, or other recording or electronic medium.

“Product insert” means the professional labeling (prescribing information) for a pharmaceutical product, a patient package insert for the pharmaceutical product, or a medication guide for the pharmaceutical product.

“Professional labeling” or “prescribing information” means the official description of a pharmaceutical product approved by a regulatory agency (e.g., FDA or EMEA) regulating marketing of the pharmaceutical product, which includes a summary of the essential scientific information needed for the safe and effective use of the drug, such as, for example indication and usage; dosage and administration; who should take it; adverse events (side effects); instructions for use in special populations (pregnant women, children, geriatric, etc.); safety information for the patient, and the like.

“Patient package insert” means information for patients on how to safely use a pharmaceutical product that is part of the FDA-approved labeling. It is an extension of the professional labeling for a pharmaceutical product that may be distributed to a patient when the product is dispensed which provides consumer-oriented information about the product in lay language, for example it may describe benefits, risks, how to recognize risks, dosage, or administration.

“Medication Guide” means an FDA-approved patient labeling for a pharmaceutical product conforming to the specifications set forth in 21 CFR 208 and other applicable regulations which contains information for patients on how to safely use a pharmaceutical product. A medication guide is scientifically accurate and is based on, and does not conflict with, the approved professional labeling for the pharmaceutical product under 21 CFR 201.57, but the language need not be identical to the sections of approved labeling to which it corresponds. A medication guide is typically available for a pharmaceutical product with special risk management information.

As used herein, “colchicine therapy” refers to medical treatment of a symptom, disorder, or condition by administration of colchicine.

“Risk” means the probability or chance of adverse reaction, injury, or other undesirable outcome arising from a medical treatment. An “acceptable risk” means a measure of the risk of harm, injury, or disease arising from a medical treatment that will be tolerated by an individual or group. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small, or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At risk” means in a state or condition marked by a high level of risk or susceptibility. Risk assessment consists of identifying and characterizing the nature, frequency, and severity of the risks associated with the use of a product.

“Safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

A “sensitive plasma concentration profile active agent” means an active agent for which a moderate change in plasma concentration can have a deleterious effect on the prescribed therapeutic intent.

“Side effect” means a secondary effect resulting from taking an active agent. The secondary effect can be a negative (unfavorable) effect or a positive (favorable) effect.

Solid dosage forms of colchicine comprise up to about 10 mg colchicine, specifically about 0.25 to about 8 mg colchicine, more specifically about 0.5 to about 4 mg colchicine, yet more specifically about 0.5 to about 1.2 mg colchicine. In an embodiment, solid dosage forms of colchicine comprise about 0.5 to about 0.6 mg colchicine. Amounts in dosage forms are given for colchicine free base, however equivalent amounts of other forms of colchicine can be used. In one embodiment, the solid dosage form is an oral dosage form, for example, a tablet.

A “substance” taken or administered with colchicine means a substance that affects the safety, bioavailability, plasma concentration, efficacy, or a combination comprising at least one of the foregoing of colchicine or the substance. A “substance” can be an active agent, an herbal supplement, a nutritional supplement, a vitamin, a xenobiotic, or an environmental contaminant.

A substance is a “substrate” of enzyme activity when it can be chemically transformed by action of the enzyme on the substance. “Enzyme activity” refers broadly to the specific activity of the enzyme (i.e., the rate at which the enzyme transforms a substrate per mg or mole of enzyme) as well as to the metabolic effect of such transformations. Thus, a substance is an “inhibitor” of enzyme activity when the specific activity or the metabolic effect of the specific activity of the enzyme can be decreased by the presence of the substance, without reference to the precise mechanism of such decrease. For example a substance can be an inhibitor of enzyme activity by competitive, non-competitive, allosteric or other type of enzyme inhibition, or other direct or indirect mechanisms. Similarly, a substance is an “activator” of enzyme activity when the specific activity or the metabolic effect of the specific activity of the enzyme can be increased by the presence of the substance, without reference to the precise mechanism of such increase. For example a substance can be an activator of enzyme activity by increasing reaction rate, by allosteric activation or other direct or indirect mechanisms. Any of these effects on enzyme activity can occur at a given concentration of active agent in a single sample, donor, or patient without regard to clinical significance. It is possible for a substance to be a substrate, inhibitor, or activator of an enzyme activity. For example, the substance can be an inhibitor of enzyme activity by one mechanism and an activator of enzyme activity by another mechanism. The function (substrate, inhibitor, or activator) of the substance with respect to activity of an enzyme can depend on environmental conditions.

A substance is a “suppressor” of observed enzyme activity in an in vitro induction study when the measured activity per unit number of cells is decreased by the presence of the substance, without reference to the precise mechanism of such decrease. For example, a substance can be a suppressor of enzyme activity in an induction study by decreasing specific activity of a fixed amount of enzyme or by decreasing enzyme level per cell for example by decreasing translation of the enzyme's mRNA or by decreasing transcription of the enzyme's gene, or by other direct or indirect mechanisms for decreasing measured enzyme activity per unit number of cells. A substance is an “inducer” of observed enzyme activity in an in vitro induction study when the measured activity per unit number of cells can be increased by the presence of the substance, without reference to the precise mechanism of such increase. For example, a substance can be an inducer of enzyme activity in the induction study by increasing specific activity of a fixed amount of enzyme, by increasing enzyme level per cell for example by increasing translation of the enzyme's mRNA or increasing transcription of the enzyme's gene, or by other direct or indirect mechanisms for increasing measured enzyme activity per unit number of cells.

A substance is a “suppressor” of enzyme expression in an in vitro induction study when the expression of the gene of the enzyme can be decreased by the presence of the substance, without reference to the precise mechanism of such decrease. For example, a substance can be a suppressor of enzyme expression by decreasing translation of the enzyme's mRNA, by decreasing transcription of the enzyme's gene, or other direct or indirect mechanisms for decreasing expression of the enzyme. A substance is an “inducer” of enzyme expression in an in vitro induction study when the expression of the gene of the enzyme can be increased by the presence of the substance, without reference to the precise mechanism of such increase. For example, a substance can be an inducer of enzyme expression by increasing translation of the enzyme's mRNA, by increasing transcription of the enzyme's gene, or other direct or indirect mechanisms for increasing expression of the enzyme.

The function (suppressor or inducer) of the substance in an in vitro induction study with respect to measured enzyme activity or expression of the gene of an enzyme can depend on environmental conditions.

A “strongly significant” result from an in vitro study means a result which is a strong indicator of a potential in vivo interaction between an active agent and another co-administered substance. In vivo evaluation of the potential interaction between the active agent and another co-administered substance can be warranted to determine whether the interaction is sufficiently large to necessitate a dosage adjustment of one or both substances, or whether the interaction would require additional therapeutic monitoring.

For an in vitro study, a strongly significant level of observed induction by the active agent of a cytochrome p450 isozyme means induction that is at least 40% of the change in induction observed for a positive control inducer of the cytochrome p450 isozyme or at least a two-fold induction of the cytochrome p450 isozyme. Specifically, for a study using cultured primary hepatocytes, this level of induction is obtained in samples from a majority of the donors tested. More specifically, this level of induction is obtained using a concentration of the active agent in the range of plasma concentrations observed in vivo after administration of the active agent or the level of observed induction shows a concentration dependent trend in the samples of each donor showing at least 40% of the change in induction observed for a positive control inducer or at least a two-fold induction of the cytochrome p450 isozyme.

Additionally, for an in vitro study, a strongly significant level of observed inhibition of a cytochrome p450 isozyme by the active agent means that the active agent reduced the activity of the enzyme by 50% or more. Specifically, reduction in activity is observed to occur in a dose dependent way to produce this level of inhibition. More specifically, this level of reduction is obtained at a concentration of the active agent in the range of plasma concentrations observed in vivo after administration of the active agent. Yet more specifically, when primary cultures of hepatocytes are used in the enzyme activity assay, the level of reduction is observed in the samples from a majority of the donors tested.

“Subtherapeutic outcome” means a response to an active agent that is less than that anticipated from a dosing regimen of the active agent used for treatment of disease or for modification of physiological function.

The terms “treating” and “treatment” mean implementation of therapy with the intention of reducing in severity or frequency symptoms, elimination of symptoms or underlying cause, prevention of the occurrence of symptoms or their underlying cause, or improvement or remediation of damage.

A “user” means a patient, a medical care worker, or a pharmaceutical supplier.

The cytochrome p450 enzymes are a highly diverse superfamily of enzymes. Each cytochrome p450 enzyme is termed an “isoform” or “isozyme” since each derives from a different gene. Cytochrome p450 enzymes are categorized into families and subfamilies by amino acid sequence similarities. These enzymes are designated by the letters “CYP” followed by an Arabic numeral representing the family, a letter representing the sub-family and another Arabic numeral representing a specific gene (e.g., CYP2D6). Particular isozymes discussed herein are named as per the recommendations of the P450 Gene Superfamily Nomenclature Committee (see e.g., “P450 superfamily: Update on new sequences, gene mapping, accession numbers, and nomenclature” Pharmacogenetics 6, 1-42 1996, part A pp. 1-21). Herein, the designation for a cytochrome p450 isozyme may encompass the homolog from any species identified as having such an isozyme. For example, CYP1A2 genes are known in at least rat, human, rabbit, hamster, dog, guinea pig, mouse, and chicken and the designation “CYP1A2” includes the CYP1A2 protein from any species known to have a CYP1A2 gene. In some embodiments, the designation for a cytochrome p450 isozyme is the human isozyme.

In one embodiment, CYP1A2 is human CYP1A2 (Entrez Gene ID: 1544; reference protein sequence Genbank NP_{—}000752), and includes any allelic variants. Specifically, CYP1A2 includes any allelic variants included in the list of human CYP1A2 allelic variants maintained by the Human Cytochrome P450 (CYP) Allele Nomenclature Committee; more specifically it includes any of the *1 through *16 alleles. Additional reference amino acid sequences for human CYP1A2 include Genbank AAK25728, AAY26399, AAA35738, AAA52163, AAA52163, AAF13599, AAH67424, AAH67425, AAH67426, AAH67427, AAH67428, AAH67429, AAA52154, AAA52146, CAA77335, P05177, Q6NWU3, Q6NWU5, Q9BXX7, and Q9UK49.

In one embodiment, CYP2A6 is human CYP2A6 (Entrez Gene ID: 1548; reference protein sequence Genbank NP_{—}000753), and includes any CYP2A6 allelic variants. Specifically, CYP2A6 includes any allelic variants included in the list of human CYP2A6 allelic variants maintained by the Human Cytochrome P450 (CYP) Allele Nomenclature Committee; more specifically it includes any of the *1 through *22 alleles. Additional reference amino acid sequences for human CYP2A6 include Genbank AAG45229, AAB40518, AAF13600, AAH96253, AAH96254, AAH96255, AAH96256, AAA52067, CAA32097, CAA32117, P11509, Q13120, and Q4VAU0.

In one embodiment, CYP2B6 is human CYP2B6 (Entrez Gene ID: 1555; reference protein sequence Genbank NP_{—}000758), and includes any CYP2B6 allelic variants. Specifically, CYP2B6 includes any allelic variants included in the list of human CYP2B6 allelic variants maintained by the Human Cytochrome P450 (CYP) Allele Nomenclature Committee; more specifically it includes any of the *1 through *25 alleles. Additional reference amino acid sequences for human CYP2B6 include Genbank AAF32444, AAD25924, ABB84469, AAF13602, AAH67430, AAH67431, AAA52144, P20813, Q6NWU1, Q6NWU2, and Q9UNX8.

In one embodiment, CYP2C8 is human CYP2C8 (Entrez Gene ID: 1558; reference protein sequence Genbank NP_{—}110518), and includes any CYP2C8 allelic variants. Specifically, CYP2B8 includes any allelic variants included in the list of human CYP2C8 allelic variants maintained by the Human Cytochrome P450 (CYP) Allele Nomenclature Committee; more specifically it includes any of the *1 through *10 alleles. Additional reference amino acid sequences for human CYP2C8 include Genbank CAH71307, AAR89907, CAA38578, AAH20596, AAA35739, AAA35740, AAA52160, AAA52161, CAA35915, CAA68550, P10632, Q5VX93, Q8WWB1, and Q9UCZ9.

In one embodiment, CYP2C9 is human CYP2C9 (Entrez Gene ID: 1559; reference protein sequence Genbank NP_{—}000762), and includes any CYP2C9 allelic variants. Specifically, CYP2C9 includes any allelic variants included in the list of human CYP2C9 allelic variants maintained by the Human Cytochrome P450 (CYP) Allele Nomenclature Committee; more specifically it includes any of the *1 through *24 alleles. Additional reference amino acid sequences for human CYP2C9 include Genbank CAH71303, AAP88931, AAT94065, AAW83816, AAD13466, AAD13467, AAH20754, AAH70317, BAA00123, AAA52159, AAB23864, P11712, Q5EDC5, Q5VX92, Q61RV8, Q8WW80, Q9UEH3, and Q9UQ59.

In one embodiment, CYP2C19 is human CYP2C19 (Entrez Gene ID: 1557; reference protein sequence Genbank NP_{—}000760), and includes any CYP2C19 allelic variants. Specifically, CYP2C19 includes any allelic variants included in the list of human CYP2C19 allelic variants maintained by the Human Cytochrome P450 (CYP) Allele Nomenclature Committee; more specifically it includes any of the *1 through *21 alleles. Additional reference amino acid sequences for human CYP2C19 include Genbank BAD02827, CAH73444, CAH74068, AAV41877, AAL31347, AAL31348, AAA36660, AAB59426, CAA46778, P33261, Q16743, Q767A3, Q8WZB1, and Q8WZB2.

In one embodiment, CYP2D6 is human CYP2D6 (Entrez Gene ID: 1565; reference protein sequence Genbank NP_{—}000097), and includes any CYP2D6 allelic variants. Specifically, CYP2D6 includes any allelic variants included in the list of human CYP2D6 allelic variants maintained by the Human Cytochrome P450 (CYP) Allele Nomenclature Committee; more specifically it includes any of the *1 through *58 alleles. Additional reference amino acid sequences for human CYP2D6 include Genbank AAS55001, ABB01370, ABB01371, ABB01372, ABB01373, AAA35737, AAA53500, BAD92729, AAU87043, AAH66877, AAH67432, AAH75023, AAH75024, AAI06758, AAI06759, CAG30316, AAA52153, AAA36403, CAA30807, and P10635.

In one embodiment, CYP2E1 is human CYP2E1 (Entrez Gene ID: 1571; reference protein sequence Genbank NP_{—}000764), and includes any CYP2E1 allelic variants. Specifically, CYP2E1 includes any allelic variants included in the list of human CYP2E1 allelic variants maintained by the Human Cytochrome P450 (CYP) Allele Nomenclature Committee; more specifically it includes any of the *1 through *7 alleles. Additional reference amino acid sequences for human CYP2E1 include Genbank CAH70047, BAA00902, BAA08796, AAA52155, AAD13753, AAF13601, CAI47002, AAH67433, AAH67435, AAZ77710, AAA35743, AAD14267, P05181, Q16868, Q5VZD5, Q6LER5, Q6NWT7, and Q6NWT9.

In one embodiment, CYP3A4 is human CYP3A4 (Entrez Gene ID: 1576; reference protein sequence Genbank NP_{—}059488), and includes any CYP3A4 allelic variants. Specifically, CYP3A4 includes any allelic variants included in the list of human CYP3A4 allelic variants maintained by the Human Cytochrome P450 (CYP) Allele Nomenclature Committee; more specifically it includes any of the *1 through *20 alleles. Additional reference amino acid sequences for human CYP3A4 include Genbank AAF21034, AAG32290, AAG53948, EAL23866, AAF13598, CAD91343, CAD91645, CAD91345, AAH69418, AAI01632, BAA00001, AAA35747, AAA35742, AAA35744, AAA35745, CAA30944, PO₅₁₈₄, P08684, Q6GRK0, Q7Z448, Q86SK2, Q86SK3, and Q9BZM0.

Various laboratory methods are known, including ones that are commercially available, for detecting the presence of allelic variants of cytochrome p450 isozymes in an individual or determining the metabolizer phenotype of an individual for a particular cytochrome p450 isozyme. Any suitable method known in the art may be used. Methods include analyzing a blood sample from the individual to determine the allelic variant of a particular cytochrome p450 isozyme gene present in the individual (for example by genotyping or haplotyping DNA or RNA from the gene using mass spectrometry, gel electrophoresis, or TAQMAN assays; or analyzing the protein sequence expressed by the gene). The metabolizer phenotype of the individual can be inferred based on the known properties of the allelic variants determined to be present in the individual. Alternatively, the blood sample can be used to measure enzyme activity of the cytochrome p450 isozyme using a suitable assay and isozyme-selective substrate. Among suitable isozyme-selective substrates are those used in the studies herein, or those suggested in publications of the United States Food and Drug Administration (FDA) directed to collecting cytochrome p450 isozyme data for regulatory submissions relating to an active agent, for example, the document “Drug Interaction Studies-Study Design, Data Analysis, and Implications For Dosing and Labeling; Draft Guidance”, dated September 2006, and available from the Center for Drug Evaluation and Research (CDER) Guidance Documents web page of the FDA website.

The ability of colchicine to affect enzyme activity of various cytochrome P450 isozymes was determined in studies described in the Examples.

In the inhibition study in which enzyme activity was determined in human liver microsomes with the simultaneous presence of colchicine and a cytochrome P450-isozyme specific substrate, colchicine inhibited CYP2A6 and CYP2C8 at a statistically significant level and activated CYP3A4 at a statistically significant level. In these experiments, colchicine was not found to affect enzyme activity of CYP1A2, CYP2B6, CYP2C8, CYP2C9, CYP2C19, CYP2D6, and CYP2E1 at a statistically significant level.

In the induction study in which enzyme activity was determined after preincubation of colchicine in the growth medium of primary cultured human hepatocytes for 48 hours, CYP1A2, CYP2A6, CYP2B6, CYP2C8, CYP2C9, CYP2C19, CYP2D6, CYP2E1, and CYP3A4 enzyme activities were not induced by colchicine. Instead, colchicine was determined to suppress enzymatic activity of each of the cytochrome P450 isozymes examined, CYP1A2, CYP2A6, CYP2B6, CYP2C8, CYP2C9, CYP2C19, CYP2D6, CYP2E1, and CYP3A4, at a statistically significant level.

Experiments were performed to look in more detail at the suppression of CYP1A2 activity by colchicine observed in the induction study. Effects of colchicine on enzyme activity levels and mRNA expression levels of CYP1A2 were determined and compared to the effects observed using vinblastine, another microtubule-binding active agent. Colchicine was observed to suppress enzyme activity by down-regulating mRNA expression, whereas vinblastine suppressed neither enzyme activity nor mRNA expression of CYP1A2.

Additionally, experiments were performed to identify cytochrome P450 isozymes that metabolize colchicine. CYP1A2, CYP2D6, and CYP2E1 showed no metabolism of colchicine. Metabolism of colchicine by CYP2A6, CYP2B6, CYP2C8, CYP2C9, CYP2C19, and CYP3A4 was observed. The cytochrome P450 isozyme showing the greatest amount of colchicine metabolism in these experiments was CYP3A4.

The invention provides methods of using colchicine. These methods include using colchicine in the treatment or prevention of various diseases or conditions in a patient, including for example, gout, attacks of acute gouty arthritis, pain in attacks of acute gouty arthritis; a cystic disease (for example polycystic kidney disease or cystic fibrosis), a lentiviral infection, demyelinating diseases of central or peripheral origin, multiple sclerosis, cancer, an inflammatory disorder such as rheumatoid arthritis, glaucoma, Dupuytren's contracture, idiopathic pulmonary fibrosis, primary amyloidosis, recurrent pericarditis, acute pericarditis, asthma, postpericardiotomy syndrome, proliferative vitreoretinopathy, Behçet's disease, Familial Mediterranean fever, idiopathic thrombocytopenic purpura, primary biliary cirrhosis, and pyoderma gangrenosum, or in enhancing bone formation or bone mineral density. Using colchicine in the treatment or prevention of a disease or condition in a patient can include administering colchicine to a patient, dispensing colchicine to a patient, or dispensing colchicine to a medical care worker for administering to a patient.

In an embodiment, the method comprises informing a user that colchicine affects the activity of a cytochrome P450 isozyme. In one embodiment, the method comprises informing a user that colchicine is metabolized by cytochrome P450 2A6, 2B6, 2C8, 2C9 or 2C19; inhibited cytochrome P450 2A6 or 2C8 enzyme activity in an in vitro inhibition study; activated CYP3A4 enzyme activity in an in vitro inhibition study; suppressed enzyme activity of cytochrome P450 1A2, 2A6, 2C19, 2D6, or 2E1 in an in vitro induction study; or suppressed mRNA expression of cytochrome P450 1A2 in an in vitro induction study. In certain embodiments the cytochrome P450 isozyme is a human enzyme. The method can further comprise providing the user with colchicine.

Informing the user that colchicine affects the activity of a cytochrome P450 isozyme includes providing a user with information about any effect of colchicine on the activity of the cytochrome P450 isozyme disclosed herein. Informing the user that colchicine affects the activity of a cytochrome P450 isozyme includes informing a user of any of the following: that colchicine is metabolized by cytochrome P450 2A6, 2B6, 2C8, 2C9 or 2C19; that colchicine inhibited cytochrome P450 2A6 or 2C8 enzyme activity in an in vitro inhibition study; that colchicine activated CYP3A4 enzyme activity in an in vitro inhibition study; that colchicine reduced enzyme activity of cytochrome P450 1A2, 2A6, 2B6, 2C8, 2C9, 2C19, 2D6, 2E1, or 3A4 in an in vitro induction study; that colchicine significantly reduced enzyme activity of cytochrome P450 1A2, 2A6, 2B6, 2C8, 2C9, 2D6, 2E1, or 3A4 in an in vitro induction study, wherein a significant reduction is at least a 50% reduction; that colchicine reduced mRNA expression of cytochrome P450 1A2 in an in vitro induction study.

The method can further comprise informing the user that administration of colchicine with a substance can affect the plasma concentration, bioavailability, safety, efficacy, or a combination comprising at least one of the foregoing of colchicine or the substance. In some embodiments, the method further comprises providing the user with the substance.

Informing the user that administration of colchicine with a substance can affect the plasma concentration, bioavailability, safety, efficacy, or a combination comprising at least one of the foregoing of colchicine or the substance includes providing a user with information about any effect of colchicine on plasma concentration, bioavailability, safety, efficacy, or a combination comprising at least one of the foregoing of colchicine or the substance. This includes informing a user of any of the following: that taking colchicine with an active agent can affect the bioavailability, safety, or efficacy of the active agent or colchicine; that administration of colchicine and a substance that is a substrate, inhibitor, activator, inducer, or suppressor of cytochrome P450 1A2, 2A6, 2B6, 2C8, 2C9, 2C19, 2D6, 2E1, or 3A4 can affect plasma concentration, bioavailability, safety, efficacy, or a combination comprising at least one of the foregoing of colchicine or the substance; that administration of colchicine and a substance that is a known substrate, inhibitor, activator, inducer, or suppressor of cytochrome P450 1A2, 2A6, 2B6, 2C8, 2C9, 2C19, 2D6, 2E1, or 3A4 can result in altered metabolism of colchicine or the substance; that administration of colchicine with a substance that is a known substrate of cytochrome P450 2A6 or 2C8 can result in reduced metabolism of the substance or increased plasma concentration of the substance; that administration of colchicine with a substance that is metabolized by CYP3A4 can result in increased metabolism of the substance or decreased plasma concentration of the substance; that administration of colchicine with a substance that is metabolized by cytochrome P450 1A2, 2A6, 2B6, 2C8, 2C9, 2C19, 2D6, 2E1, or 3A4 can result in decreased metabolism of the substance or increased plasma concentration of the substance; that administration of colchicine with a substance that is a known substrate of cytochrome P450 1A2 can result in reduced metabolism of the substance or increased plasma concentration of the substance; that administration of colchicine with a substance that is metabolized by cytochrome P450 1A2, 2A6, 2B6, 2C8, 2C9, 2C19, 2D6, 2E1, or 3A4 can affect plasma concentration, bioavailability, safety, efficacy, or a combination comprising at least one of the foregoing of the substance or colchicine; that administration of colchicine with a substance that is a substrate or an inhibitor of cytochrome P450 2A6, 2B6, 2C8, 2C9, 2C19, or 3A4 can result in reduced metabolism of colchicine or increased plasma concentration of colchicine; that caution is recommended when administering colchicine with a substance, wherein the substance is an active agent that has a sensitive plasma concentration profile or a narrow therapeutic index; that there is a potential active agent interaction between colchicine and a substance that is a substrate of cytochrome P450 1A2, 2A6, 2B6, 2C8, 2C9, 2C19, 2D6, 2E1, or 3A4; that there is a potential active agent interaction between colchicine and a substance that is an inhibitor, activator, suppressor, or inducer of cytochrome P450 2A6, 2B6, 2C8, 2C9, or 2C19; that there is a potential active agent interaction between colchicine and a substance that is a substrate of cytochrome P450 1A2, 2A6, 2C19, 2D6, or 2E1; that caution is recommended when colchicine and a substrate of CYP2A6, CYP2B6, CYP2C9, 2C19, or 2D6 are administered to a patient having a poor metabolizer phenotype for or reduced activity of the cytochrome P450 isozyme; that the allelic variants of CYP2A6, CYP2B6, CYP2C9, 2C19, or 2D6 present in the patient can further affect a potential active agent interaction between colchicine and a substance; or that there is a potential active agent interaction of colchicine with theophylline, warfarin, or phenyloin.

The effect of administration of colchicine with the substance can be determined by comparison of the plasma concentration, bioavailability, safety, efficacy, or a combination comprising at least one of the foregoing of the substance with and without administration of colchicine or by comparison of the plasma concentration, bioavailability, safety, efficacy, or a combination comprising at least one of the foregoing of colchicine with and without administration of the substance.

In some embodiments, the method of using colchicine can further comprise administering colchicine or a substance. Administration may be to a patient by the patient, a medical care worker, or other user. Colchicine can be administered in a therapeutically effective amount. The substance can be an active agent. The active agent can have a sensitive plasma concentration profile or a narrow therapeutic index. The method can also comprise monitoring a patient, for example, monitoring the patient for an adverse reaction, a side effect, a subtherapeutic outcome, or a symptom of an active agent interaction or monitoring a patient's plasma concentration of colchicine or the substance. The method can also comprise adjusting administration or dosing of the substance or colchicine for the patient based on the results of monitoring, for example, a determined plasma concentration of the active agent or colchicine.

In all of the embodiments herein, a medical care worker can determine the plasma concentration of a substance such as an active agent, including colchicine, by performing or ordering the performance of any suitable method. For example, the medical care worker could order a test using blood drawn from the patient for determining the plasma concentration of colchicine or the substance.

Medical information provided in any of the methods described herein concerning the effects of administering colchicine with an additional substance may alternatively be provided in layman's terms, so as to be better understood by patients or non-medical professionals. Those of skill in the medical art are familiar with the various layman's terms that can be used to describe the effects of active agent interactions.

In an embodiment, the method of using colchicine comprises obtaining colchicine from a container associated with published material providing information that colchicine affects the activity of a cytochrome P450 isozyme. Information can also be provided that administering colchicine with a substance can affect plasma concentration, bioavailability, safety, efficacy, or a combination comprising at least one of the foregoing of the substance or colchicine. The information provided by the published material can comprise any combination of any information disclosed herein concerning the effects of colchicine on the activity or expression of a cytochrome P450 isozyme or any information disclosed herein concerning the effects of colchicine when administered with a substance on the plasma concentration, bioavailability, safety, efficacy, or a combination comprising at least one of the foregoing of the substance or colchicine. The method can also comprise providing colchicine in the container providing such information. The method can further comprise ingesting the colchicine or the substance.

In an embodiment, the method comprises determining for a patient to whom colchicine is going to be administered or is being administered whether a substance that is currently being or will be administered to the patient is a substrate of CYP1A2; and determining risk for the patient of an adverse event during coadministration of colchicine and the substance resulting from reduced metabolism of the substance by CYP1A2.

Depending on the determined risk of an adverse event, such as an active agent-related toxicity or a subtherapeutic outcome, the methods can further comprise administering colchicine or the substance to the patient. For example, if there is no risk of an adverse event, or if the risk is determined to be acceptable, colchicine and the substance can be administered to the patient. Alternatively, if there is a risk of an adverse event, or if the risk is determined to be unacceptable, either colchicine can be administered to the patient but not the substance, or the substance can be administered to the patient but not colchicine.

The method can further comprise determining that the patient has a poor metabolizer phenotype for CYP2A6, CYP2C19, or CYP2D6 or determining that the patient belongs to an ethnic group in which there is a high frequency of a poor metabolizer phenotype of CYP2A6, CYP2C19, or CYP2D6, e.g., for CYP2C19, an Asiatic or Oceanic ethnic group.

Determining risk of an adverse reaction, such as a toxicity or a subtherapeutic outcome, resulting from coadministration of colchicine and a substance is based on an appropriate set of risk parameters. As will be evident to those of skill in the art, the risk parameters to be considered will be based upon factors which influence the risk that a known or suspected adverse reaction will occur if the patient receives colchicine with or without the substance, and will vary depending upon the substance in question for coadministration with colchicine. Factors that may define the relevant risk parameters include effect of the substance or colchicine on activity of the relevant cytochrome P450 isozyme(s), e.g. CY3A4 or CYP1A2; the likelihood that certain preexisting conditions may exist in the patient; information collected from the patient including information relating to the patient's conduct; the patient's past or ongoing medical treatment, such as other procedures or medication which the patient may have received or is still receiving; results of certain diagnostic tests which have been performed; and the like. For example, if the substance is theophylline, risk factors identified as reducing theophylline clearance include the age of the patient, whether or not the patient is a smoker, and whether the patient has any of the following concurrent diseases or conditions: acute pulmonary edema, congestive heart failure, cor-pulmonale, fever, hypothyroidism, liver disease (e.g., cirrhosis or acute hepatitis), sepsis with multi-organ failure, and shock. Information collected from the patient for determining risk may be obtained prior to the initial dispensation of colchicine or the substance to the patient or may be obtained from the patient on a periodic basis. For example, after treatment with colchicine and the substance is begun, information on the onset of certain symptoms which may be indicative of the need for changes in the patient's treatment regimen may be obtained from the patient on a periodic basis. For example if colchicine and theophylline are coadministered, information on development of nausea or vomiting, particularly repetitive vomiting, or other signs or symptoms consistent with theophylline toxicity should be obtained.

Determining risk can comprise accessing a computer-hosted database to obtain information relevant to assessing risk, for example adverse reactions associated with an active agent, active agent interactions, risk factors for an adverse reaction in administration of an active agent, dosing, and the like. The database may be in the form of a look-up table, or similar structure, that provides output information based on the input of information. The database can also be a component of a pharmacy management system.

Pharmacy management systems are computer-based systems that are widely used by commercial pharmacies to manage prescriptions and to provide pharmacy and medical personnel with warnings and guidance regarding active agents being administered to individuals. Such systems typically provide alerts warning either or both of medical care providers and patients when an active agent that may be harmful to the particular patient is prescribed. For example, such systems can provide alerts warning that a patient has an allergy to a prescribed active agent, or is receiving concomitant administration of an active agent that can have a dangerous interaction with a prescribed active agent. U.S. Pat. Nos. 5,758,095, 5,833,599, 5,845,255, 6,014,631, 6,067,524, 6,112,182, 6,317,719, 6,356,873, and 7,072,840, each of which is incorporated herein by reference, disclose various pharmacy management systems and aspects thereof. Many pharmacy management systems are now commercially available, e.g., CENTRICITY Pharmacy from BDM Information Systems Ltd., General Electric Healthcare, Waukesha, Wis., Rx30 Pharmacy Systems from Transaction Data Systems, Inc., Ocoee, Fla., SPEED SCRIPT from Digital Simplistics, Inc., Lenexa, Kans., and various pharmacy management systems from OPUS-ISM, Hauppauge, N.Y.

Alternatively, determining risk can comprise obtaining information relevant to assessing risk from standard treatment guidelines, textbooks, compendial literature, journals, drug manufacturer guidelines, internet websites providing information on active agent interactions (e.g., “Drug Interaction Checker” at the MEDScape website or the drug interaction website maintained by Dr. D. Flockhart, Indiana University School of Medicine); or FDA requirements for particular active agents.

Diagnostic tests may be probative of the concentration of one or more active agents, including a prescribed active agent, to assure that appropriate dosing is maintained in the patient. Such diagnostic testing may be conducted on any bodily fluid or waste product of the patient, including the blood, serum, plasma, saliva, semen or urine, as well as the feces. Diagnostic testing may also be performed on a biopsy of any tissue of the patient or may include genetic testing, which may be indicative of a genetic predisposition to a particular adverse side effect. Other forms of diagnostic testing, such as diagnostic imaging, or tests which may be probative of the proper functioning of any tissue, organ, or system are also contemplated. Preferably, appropriate information or diagnostic test results are obtained and considered in determining risk.

In an embodiment, the method comprises administering colchicine to a patient; and monitoring the patient during administration of colchicine if the patient is taking a substance that is a known substrate of cytochrome P450 1A2, 2A6, 2C19, 2D6, or 2E1. Adjusting administration of colchicine or the substance to the patient to avoid an adverse event in the patient can be performed.

In an embodiment, the method comprises determining that a substance that is a substrate of cytochrome P450 1A2, 2A6, 2C19, 2D6, or 2E1 is administered to the patient; and adjusting administration of colchicine or the substance to the patient to avoid an adverse reaction.

In an embodiment, the method comprises determining that colchicine reduced enzyme activity of cytochrome P450 1A2, 2A6, 2C19, 2D6, or 2E1 in an in vitro induction study; or reduced mRNA expression of cytochrome P450 1A2 in an in vitro induction study; and monitoring the patient during administration of colchicine if a substance that is a substrate of cytochrome P450 1A2, 2A6, 2C19, 2D6, or 2E1 is coadministered to the patient. The method can further comprise determining that a substance that is a substrate of cytochrome P450 1A2, 2A6, 2C19, 2D6, or 2E1 is coadministered to the patient or adjusting administration of colchicine or the substance to the patient to avoid an adverse reaction.

Such methods can include informing the patient receiving a substance or the patient's medical care worker that administration of colchicine with a substance can affect the plasma concentration, bioavailability, safety, efficacy, or a combination comprising at least one of the foregoing of colchicine or the substance. The method can include informing the patient receiving a substance or the patient's medical care worker of any information disclosed herein about the effects of colchicine on cytochrome P450s and any information disclosed herein about the effect of colchicine or the substance on the plasma concentration, bioavailability, safety, efficacy, or a combination comprising at least one of the foregoing of colchicine or the substance when colchicine is used with the substance.

Determining that a substance that is a known substrate, inhibitor, or inducer of a particular cytochrome P450 isozyme is administered to a patient in need of colchicine therapy can be performed by consulting with the patient regarding substances, e.g., medications, taken in by the patient; a medical care worker administering medications to the patient; a prescription database including medications prescribed to the patient; or by any other method known in the art.

Determining that colchicine is metabolized by cytochrome P450 2A6, 2B6, 2C8, 2C9 or 2C19; inhibited cytochrome P450 2A6 or 2C8 enzyme activity in an in vitro inhibition study; activated CYP3A4 enzyme activity in an in vitro inhibition study; suppressed enzyme activity of cytochrome P450 1A2, 2A6, 2C19, 2D6, or 2E1 in an in vitro induction study; or suppressed mRNA expression of cytochrome P450 1A2 in an in vitro induction study or determining that co-administration of colchicine and a substance that is a substrate of cytochrome P450 1A2, 2A6, 2C19, 2D6, or 2E1 may result in an increased plasma concentration of the substance, or that co-administration of colchicine and a substance that is a substrate of cytochrome P450 1A2, 2A6, 2C19, 2D6, or 2E1 may result in decreased metabolism of the substance can be performed by consulting the package insert for the colchicine product or for the substance administered to the patient; consulting a database including prescribing information and potential risks for colchicine or the substance, e.g., a pharmacy management system; or by any other method known in the art.

Monitoring the patient can comprise monitoring the patient's plasma concentration of colchicine or the substance; monitoring the patient for symptoms of an active agent interaction between the substance and colchicine; monitoring the patient for an adverse reaction (e.g., a toxicity or a subtherapeutic outcome) resulting from administration of the substance and colchicine; monitoring the patient for an adverse reaction (e.g., a toxicity or a subtherapeutic outcome) associated with colchicine; monitoring the patient for decreased efficacy of colchicine; monitoring the patient for an adverse reaction associated with elevated plasma concentration of the substance; monitoring the patient for an adverse reaction or side effect associated with the substance; monitoring the patient for a substance-associated toxicity; or monitoring the patient for a symptom of elevated plasma concentration of the substance.

Monitoring the patient can be monitoring any appropriate patient-specific, disease-specific, or substance-specific parameter appropriate to avoid or safely manage an active agent interaction. Monitoring the patient can be, for example, monitoring the patient for an adverse reaction, a subtherapeutic outcome, a side effect, or a symptom of an active agent interaction for example by physical examination or visual identification; monitoring the blood level of colchicine or the substance in the patient; monitoring clinical laboratory tests appropriate for colchicine, the substance, or a medical diagnosis for the patient; monitoring therapeutic effect of colchicine or the substance on the patient's condition; monitoring occurrence in the patient of a known side effect or adverse reaction of colchicine or the substance; monitoring the patient for a symptom of an active agent interaction between the substance and colchicine; monitoring the patient for an adverse reaction or side effect associated with altered plasma concentration of colchicine or the substance; monitoring the patient for occurrence of an unexpected response during treatment; monitoring changes in control, signs, or symptoms of a condition of the patient, or determining a complete list of medical diagnoses for the patient. Monitoring the patient can be performed by the patient or by a medical care worker.

Most active agents have adverse side effects having widely variable incidence, according to individual sensitivity. For colchicine, the most frequently reported adverse reactions to colchicine therapy are abdominal pain with cramps, diarrhea, nausea, and vomiting. Less frequently or rarely reported adverse reactions associated with colchicine therapy include anorexia, agranulocytosis, allergic dermatitis, allergic reactions, alopecia, angioedema, aplastic anemia, bone marrow depression, myopathy, neuropathy, skin rash, thrombocytopenic disorder, and urticaria.

Determining that a patient experiences an adverse reaction can be performed by obtaining information from the patient regarding onset of certain symptoms which may be indicative of the adverse reaction, results of diagnostic tests indicative of the adverse reaction, and the like.

Determining the level of metabolism of a substance or colchicine in a subject may be performed for example by determining plasma concentrations of colchicine or the substance or of an appropriate metabolite of colchicine or the substance, or any other methods known in the art.

Adjusting administration of colchicine or the substance to the patient to avoid an adverse reaction or a subtherapeutic outcome, or adjusting dosing regimens can be performed by one of ordinary skill in the art, taking into consideration the physiology of the patient, including such factors as the age, sex, and health of the patient, as well as active agents the patient may be taking at the time. Optionally, the patient can be monitored at the initial, or a subsequent, stage of treatment to ensure therapeutic plasma levels of colchicine or the substance are achieved or maintained.

In another aspect, herein disclosed are methods for using colchicine which methods involve the use of pharmacy management systems.

In one aspect, one such method comprises a pharmacy receiving a prescription for colchicine for a patient who is suffering from a condition treatable with colchicine and who is concomitantly being treated with a second active agent that is a substrate of CYP1A2, followed by the pharmacy dispensing colchicine in response to receipt of the prescription, wherein the dispensing is preceded by entry into a first computer readable storage medium, in functional communication with a computer, of a unique patient identifier for said patient and at least one active agent identifier for colchicine linked to the patient identifier so as to indicate that colchicine is to be administered to the patient. The computer is programmed to issue an active agent interaction alert when the at least one active agent identifier for colchicine is entered linked to the patient identifier so as to indicate that colchicine is to be administered to the patient and when the patient identifier is also linked to an identifier indicating that a second active agent that is a substrate of CYP1A2 is being concomitantly administered to the patient. Upon entry of the at least one active agent identifier for colchicine linked to the patient identifier, an active agent interaction alert is issued to one or more of a pharmacy technician, a pharmacist, or a pharmacy customer obtaining the colchicine, said alert indicating that a second active agent that is a substrate of CYP1A2 is being concomitantly administered to the patient and that prior to the colchicine being dispensed, the prescribed colchicine and second active agent dosing regimens must be reviewed and, if necessary adjusted by the prescribing medical care worker.

The active agent interaction alert is preferably issued as one or both of a written warning on a display screen of the pharmacy management computer system, and a printed warning. The printed warning may be attached to or packaged with the dispensed prescription.

Methods of using colchicine include methods in which the user is a patient in need of treatment with colchicine and additionally comprising administering colchicine and an active agent to the patient. The patient in need of treatment with colchicine may be, for example, a human patient, a patient in need of treatment of attacks of acute gouty arthritis and pain in attacks of acute gouty arthritis, a cystic disease, for example polycystic kidney disease or cystic fibrosis, a lentiviral infection, demyelinating diseases of central or peripheral origin, multiple sclerosis, cancer, an inflammatory disorder such as rheumatoid arthritis, glaucoma, Dupuytren's contracture, idiopathic pulmonary fibrosis, primary amyloidosis, recurrent pericarditis, acute pericarditis, asthma, postpericardiotomy syndrome, proliferative vitreoretinopathy, Behçet's disease, Familial Mediterranean fever, idiopathic thrombocytopenic purpura, primary biliary cirrhosis, and pyoderma gangrenosum, or in enhancing bone formation or bone mineral density, a patient receiving prophylactic colchicine treatment, or a patient undergoing colchicine therapy. The active agent administered to the patient with colchicine can be for treatment or prophylaxis of a condition of the patient other than the condition needing treatment with colchicine. The amount of colchicine or the active agent administered may be a therapeutically effective amount.

In an embodiment, a method can additionally include monitoring the patient's plasma concentration of the active agent or colchicine. When colchicine is administered together with another active agent, methods of use can include determining the plasma concentration of the active agent or colchicine and adjusting the dosing regimen of the active agent or colchicine for the patient based on the determined plasma concentration of the active agent or colchicine.

When the substance administered with colchicine is an NTI or sensitive plasma concentration profile active agent, methods using a blood test to monitor plasma levels of the NTI or sensitive plasma concentration profile active agent comprise administering to a patient colchicine and the NTI or sensitive plasma concentration profile active agent, and monitoring the blood levels of the NTI or sensitive plasma concentration profile active agent. Methods can also include adjusting dosing of the NTI or sensitive plasma concentration profile active agent for the patient based on the determined plasma concentration of the active agent.

In some embodiments, the NTI active agent comprises warfarin. Warfarin, 3-(a-acetonylbenzyl)-4-hydroxycoumarin, is an anticoagulant, which is eliminated by metabolism by cytochrome p450 isoforms including CYP2C9, CYP2C19, CYP2C8, CYP2C18, CYP1A2, and CYP3A4. Warfarin has a narrow therapeutic index such that too little can lead to excessive clotting, while excessive warfarin can lead to excessive bleeding. The dosing of warfarin is individualized according to the patient's sensitivity to the active agent as indicated, for example, by the Prothrombin Time/International Normalized Ratio (PT/INR). The PT/INR gives an indication of how fast blood is clotting. The recommended initial dose is 2-5 mg/day, with 2-10 mg/day as the maintenance dose. Warfarin tablets for oral administration include tablets comprising 1, 2, 2.5, 3, 4, 5, 6, 7.5, and 10 mg of warfarin. The INR may be adjusted to 2.0-4.5, or 2.0-3.0 or 2.5-3.5 depending on whether the warfarin is being administered to treat venous thromboembolism, non-valvular atrial fibrillation, post-myocardial infarction, heart valve prophylaxis, or recurrent systemic embolism.

In the PT test, a reagent which induces coagulation is added to a sample of the patient's plasma. The reagent typically primarily comprises thromboplastin and calcium chloride. Many commercially available PT reagents contain crude thromboplastin extracted from natural sources, e.g., rabbit brain, rabbit brain/lung mixtures, human placenta, or bovine brain, although recombinant thromboplastin may also be employed. Prothrombin time assays are performed by mixing the plasma sample and reagent at a constant temperature such as 37° C., and monitoring the progress of the reaction until a perceptible clot (or “gel clot”) is detected. The development of a gel clot is the end point of the reaction. This end point may be detected in various ways such as by viscosity change, by electrode reaction, and, most commonly, by photometric means. The test result is generally compared to a result using a normal (control) plasma and converted to an INR.

The International Normalized Ratio, or INR, was developed to standardize PT values, so that test results from different thromboplastins and coagulation analyzers become equivalent. Under the INR system, a thromboplastin is assigned an International Sensitivity Index (ISI) value. The ISI indicates the relative sensitivity of the thromboplastin compared to an international reference thromboplastin. If a thromboplastin has the same sensitivity as the reference thromboplastin, then its ISI is 1.0. A higher ISI value indicates that a thromboplastin is less sensitive than the reference thromboplastin. The ISI is used in the following formula to calculate an INR value from a PT value: INR=(patient PT/mean normal PT)ISI. The ISI is usually determined by the thromboplastin manufacturer. Different ISI values are assigned for different models or classes of coagulation analyzers.

In an embodiment of the method of using colchicine in which the substance is warfarin, the method comprises administering to a patient colchicine and warfarin, and monitoring the blood levels of warfarin and colchicine or monitoring the Prothrombin Time/International Normalized Ratio.

In another embodiment, the method comprises administering colchicine and warfarin to a patient in need of colchicine and an anticoagulant, and monitoring the Prothrombin Time/International Normalized Ratio. Monitoring the Prothrombin Time/International Normalized Ratio may be performed daily, every other day, weekly, every other week, or monthly, for example. The method may further comprise providing to the patient or medical care worker instructions regarding measuring the Prothrombin Time/International Normalized Ratio daily, every other day, weekly, every other week, monthly, or according to another schedule or time criteria.

The NTI active agent can also comprise phenyloin. Phenyloin, 5,5-diphenylhydantoin, is an antiepileptic active agent useful in the treatment of epilepsy which is eliminated by metabolism by cytochrome P450 isoforms including CYP1A2, CYP2C9, CYP2C19, and CYP3A4. Phenyloin has a narrow therapeutic index such that too little can lead to insufficient results and excessive phenyloin can lead to phenyloin toxicity. The typical clinically effective serum level is about 10 to about 20 mg/mL. The recommended initial dose is one 100 mg capsule 3 to 4 times per day, with 300 mg/day dose in three divided doses or one single dose per day. The dosing of phenyloin can be individualized according to the patient's sensitivity to the active agent by measuring plasma concentration of phenyloin.

In an embodiment of the method of using colchicine in which the substance is phenyloin, the method comprises administering colchicine and phenyloin to a patient in need of colchicine and an antiepileptic, and monitoring the blood levels of phenyloin.

The NTI active agent can also comprise theophylline. Theophylline is a bronchodilator structurally classified as a xanthine derivative. Theophylline is a substrate of CYP1A2 and CYP2E1. Adverse reactions associated with theophylline are generally mild when peak serum theophylline concentrations are less than about 20 μg/mL and mainly consist of transient caffeine-like adverse effects such as nausea, vomiting, headache, and insomnia. When peak serum theophylline concentrations exceed 20 μg/mL, however, theophylline produces a wide range of adverse reactions including persistent vomiting, cardiac arrhythmias, and intractable seizures which can be lethal. The dose of theophylline must be individualized on the basis of peak serum theophylline concentration measurements in order to achieve a dose that will provide maximum potential benefit with minimal risk of adverse events.

In an embodiment, the method of using colchicine when the substance is theophylline comprises administering colchicine and theophylline to a patient in need of colchicine and a bronchodilator; monitoring the blood levels of theophylline; and adjusting dosing of theophylline to avoid an adverse reaction.

Also disclosed herein are methods of manufacturing a colchicine pharmaceutical product.

In one embodiment, the method comprises packaging a colchicine dosage form with published material providing information on the effects of colchicine on a cytochrome p450 isozyme. The information can include any information disclosed herein concerning colchicine effects on a cytochrome p450 isozyme. The information can also include any information disclosed herein about the effects of administering colchicine and a substance on the plasma concentration, bioavailability, safety, efficacy, or a combination comprising at least one of the foregoing of colchicine or the substance when the substance is used with colchicine.

The invention provides articles of manufacture.

In some embodiments, the article of manufacture comprises a container containing a dosage form of colchicine and labeling or published material, e.g., as a product insert or a patient package insert. The published material can indicate quantities of the components to be administered, guidelines for administration, safety issues, and the like.

In some embodiments, the container is associated with published material informing that colchicine affects a cytochrome p450 isozyme. The information provided by the published material can include any information disclosed herein concerning effects of colchicine on a cytochrome p450 isozyme. The published material comprised in the article of manufacture can also include any information disclosed herein concerning the effect of administering colchicine and a substance on the plasma concentration, bioavailability, safety, efficacy, or a combination comprising at least one of the foregoing of colchicine or the substance when the substance is used with colchicine. The published material may be in the form of printed labeling, or in some other form.

Also disclosed herein is an article of manufacture comprising packaging material and a dosage form contained within the packaging material, wherein the dosage form comprises colchicine, and wherein the packaging material comprises a label approved by a regulatory agency for the product. Examples of regulatory agencies are the US FDA or the European Agency for the Evaluation of Medicinal Products (EMEA). The label can inform of any information disclosed herein about the effect of colchicine on metabolism by a cytochrome p450 isozyme or any information disclosed herein about the effects of administering colchicine and a substance on the plasma concentration, bioavailability, safety, efficacy, or a combination comprising at least one of the foregoing of colchicine or the substance when the substance is used with colchicine.

In embodiments of the articles of manufacture, the dosage form will typically be contained in a suitable container capable of holding and dispensing the dosage form and which will not significantly interact with the active agent(s) in the dosage form. Further, the container will be in physical relation with the published material. The published material may be associated with the container by any means that maintains physical proximity of the two. By way of example, the container and the published material can both be contained in a packaging material such as a box or plastic shrink wrap. Alternatively, the published material can be bonded to the container, such as with glue that does not obscure the published material, or with other bonding or holding means. Yet another alternative is that the published material is placed within the container with the dosage form.

Someone can also hand the published material to the patient, for example a pharmacist can hand a product insert, patient package insert, or medication guide to a patient in conjunction with dispensing the dosage form. The published material may be a product insert, patient package insert, medication guide, flyer, brochure, or a packaging material for the dosage form such as a bag, or the like.

In any of the embodiments disclosed herein the published material or information associated with or provided by a container can be contained in any fixed and tangible medium. For example, the information can be part of a leaflet, brochure, or other printed material provided with a container or separate from a container. The information can also take the form of a flyer, advertisement, or the label for marketing the active agent approved by a regulatory agency. The information can also be recorded on a compact disk, DVD or any other recording or electronic medium.

The container can be in the form of bubble or blister pack cards, optionally arranged in a desired order for a particular dosing regimen. Suitable blister packs that can be arranged in a variety of configurations to accommodate a particular dosing regimen are well known in the art or easily ascertained by one of ordinary skill in the art.

Colchicine dosage forms existing as liquids, solutions, emulsions, or suspensions can be packaged in a container for convenient dosing of pediatric or geriatric patients. For example, prefilled droppers (such as eye droppers or the like), prefilled syringes, and similar containers housing the liquid, solution, emulsion, or suspension form are contemplated.

Colchicine can be formulated as a dosage form for administration where the formulation generally contains colchicine and a pharmaceutically acceptable excipient. As used herein, “pharmaceutically acceptable excipient” means any other component added to the pharmaceutical formulation other than the active agent. Excipients may be added to facilitate manufacture, enhance stability, control release, enhance product characteristics, enhance bioavailability, enhance patient acceptability, etc. Pharmaceutical excipients include carriers, fillers, binders, disintegrants, lubricants, glidants, compression aids, colors, sweeteners, preservatives, suspending agents, dispersing agents, film formers, flavors, printing inks, buffer agents, pH adjusters, preservatives etc.

The substance used with colchicine in the methods and articles of manufactures described herein may have certain effects, direct or indirect, on the activity of a cytochrome P450 enzyme.

In any of the above methods or articles, the substance can be an active agent.

Examples of substrates of CYP1A2 include aminophylline, amitriptyline, caffeine, clomipramine, clozapine, cyclobenzaprine, estradiol, fluvoxamine, haloperidol, imipramine, mexiletine, naproxen, olanzapine, ondansetron, phenacetin, acetaminophen, propranolol, riluzole, ropivacaine, tacrine, theophylline, tizanidine, verapamil, (R)-warfarin, zileuton, and zolmitriptan. Examples of inhibitors of CYP1A2 include amiodarone, cimetidine, a fluoroquinolone (e.g., ciprofloxacin, gatifloxacin, levofloxacin, lomefloxacin, moxifloxacin, norfloxacin, or ofloxacin), fluvoxamine, furafylline, interferon, methoxsalen, and mibefradil. Examples of inducers of CYP1A2 include chemicals released from digestion of broccoli, brussel sprouts, and char-grilled meat; chemicals inhaled when smoking tobacco; insulin, methyl cholanthrene, modafinil, nafcillin, beta-naphthoflavone, and omeprazole.

Examples of substrates of CYP2A6 include aflatoxin B₁, cotinine, coumarin, 1,7-dimethylxanthine, disulfuram, fadrozole, halothane, losigamone, letrozole, methoxyflurane, nicotine, tobacco-specific nitrosamines, SM-12502, tegafur, and valproic acid. Examples of inhibitors of CYP2A6 include tranylcypromine, methoxsalen, pilocarpine, and tryptamine. Examples of inducers of CYP2A6 include dexamethasone and pyrazole.

Examples of substrates of CYP2B6 include bupropion, cyclophosphamide, efavirenz, ifosfamide, and methadone. Examples of inhibitors of CYP2B6 include thiotepa and ticlopidine. Examples of inducers of CYP2B6 include phenobarbital and rifampin.

Examples of substrates of CYP2C8 include amodiaquine, cerivastatin, paclitaxel, repaglinide, and torsemide. Examples of inhibitors of CYP2C8 include quercetin, a glitazone (e.g., rosiglitazone or pioglitazone), gemfibrozil, montelukast, and trimethoprim. Examples of inducers of CYP2C8 include rifampin.

Examples of substrates of CYP2C9 include diclofenac, ibuprofen, meloxicam, S-naproxen, piroxicam, suprofen, tolbutamide, glipizide, losartan, irbesartan, glyburide (glibenclamide), glipizide, glimepiride, amitriptyline, celecoxib, fluoxetine, fluvastatin, nateglinide, phenyloin, rosiglitazone, tamoxifen, torsemide, and S-warfarin. Examples of inhibitors of CYP2C9 include amiodarone, fenofibrate, fluconazole, fluvastatin, fluvoxamine, isoniazid, lovastatin, phenylbutazone, probenicid, sertraline, sulfamethoxazole, sulfaphenazole, teniposide, voriconazole, and zafirlukast. Examples of inducers of CYP2C9 include rifampin and secobarbital.

Examples of substrates of CYP2C19 include the proton pump inhibitors: lansoprazole, omeprazole, pantoprazole, and E-3810; the anti-epileptics: diazepam, phenyloin, fosphenyloin, S-mephenyloin, and phenobarbitone (Phenobarbital); as well as amitriptyline, carisoprodol, citalopram, clomipramine, cyclophosphamide, hexobarbital, imipramine, indomethacin, R-mephobarbital, moclobemide, nelfinavir, nilutamide, primidone, progesterone, proguanil, propranolol, teniposide, and R-warfarin. Examples of inhibitors of CYP2C19 include chloramphenicol, cimetidine, felbamate, fluoxetine, fluvoxamine, indomethacin, ketoconazole, lansoprazole, modafinil, omeprazole, oxcarbazepine, probenicid, ticlopidine, and topiramate. Examples of inducers of CYP2C19 include carbamazepine, norethindrone, prednisone, and nifampin (rifampicin).

Examples of substrates of CYP2D6 include carvedilol, S-metoprolol, propafenone, timolol; amitriptyline, clomipramine, desipramine, imipramine, paroxetine; haloperidol, perphenazine, risperidone, thioridazine; alprenolol, amphetamine, aripiprazole, atomoxetine, bufuralol, chlorpheniramine, chlorpromazine, codeine, debrisoquine, dexfenfluramine, dextromethorphan, duloxetine, encamide, flecamide, fluoxetine, fluvoxamine, lidocaine, metoclopramide, methoxyamphetamine, mexiletine, minaprine, nebivolol, nortriptyline, ondansetron, perhexyline, phenacetin, phenformin, propranolol, sparteine, tamoxifen, tramadol, and venlafaxine. Examples of inhibitors of CYP2D6 include amiodarone, bupropion, celecoxib, chlorpromazine, chlorpheniramine, cimetidine, citalopram, clomipramine, cocaine, doxepin, doxorubicin, duloxetine, escitalopram, fluoxetine, halofantrine, red-haloperidol, levomepromazine, metoclopramide, methadone, mibefradil, midodrine, moclobemide, paroxetine, quinidine, ranitidine, ritonavir, sertraline, terbinafine, ticlopidine, histamine H1 receptor antagonists, diphenhydramine, chlorpheniramine, clemastine, perphenazine, hydroxyzine, and tripelennamine. Examples of inducers of CYP2D6 include rifampicin and dexamethasone.

Examples of substrates of CYP2E1 include enflurane, halothane, isoflurane, methoxyflurane, sevoflurane; acetaminophen, aniline, benzene, chlorzoxazone, ethanol, N,N-dimethyl formamide, and theophylline. Examples of inhibitors of CYP2E1 include diethyl-dithiocarbamate and disulfuram. Examples of inducers of CYP2E1 include ethanol and isoniazid.

Examples of substrates of CYP3A4 include clarithromycin, erythromycin, telithromycin: quinidine; alprazolam, diazepam, midazolam, triazolam; cyclosporine, tacrolimus (FK506); indinavir, nelfinavir, ritonavir, saquinavir; cisapride; astemizole, chlorpheniramine, terfenadine; amlodipine, diltiazem, felodipine, lercanidipine, nifedipine, nisoldipine, nitrendipine, verapamil; atorvastatin, cerivastatin, lovastatin, simvastatin; estradiol, hydrocortisone, progesterone, testosterone; alfentanyl, aripiprazole, buspirone, cafergot, caffeine, cilostazol, cocaine, codeine, dapsone, dextromethorphan, docetaxel, domperidone, eplerenone, fentanyl, finasteride, gleevec, haloperidol, irinotecan, Levo-Alpha Acetyl Methadol (LAAM), lidocaine, methadone, nateglinide, odanestron, pimozide, propranolol, quinine, salmeterol, sildenafil, sirolimus, tamoxifen, taxol, terfenadine, trazodone, vincristine, zaleplon, and zolpidem. Examples of inhibitors of CYP3A4 include HIV Antivirals (e.g., delavirdine, indinavir, nelfinavir, and ritonavir); amiodarone, aprepitant, cinchloramphenicol, cimetidine, clarithromycin, diethyl-dithiocarbamate, diltiazem, erythromycin, fluconazole, fluvoxamine, gestodene, grapefruit juice, Seville orange juice, imatinib, itraconazole, ketoconazole, mifepristone, nefazodone, norfloxacin, norfluoxetine, mibefradil, star fruit, verapamil, and voriconazole. Examples of inducers of CYP3A4 include HIV Antivirals (e.g., efavirenz, and nevirapine); barbiturates (e.g., allobarbital, amobarbital, aprobarbital, alphenal, barbital, brallobarbital, mephobarbital, secobarbital, and phenobarbital), carbamazepine, efavirenz, glucocorticoids (e.g., prednisone, prednisilone, methylprednisilone, dexamethasone, and hydrocortisone), modafinil, nevirapine, phenyloin, rifampin, St. John's wort, troglitazone, oxcarbazepine, pioglitazone, and rifabutin.

In any of the embodiments described herein, the substance can be a sensitive plasma concentration profile active agent. Examples of a sensitive plasma concentration profile active agent include cyclophosphamide, efavirenz, fosphenyloin, glimepiride, mexiletine, phenyloin, progesterone, tamoxifen, theophylline, warfarin, and any active agent having a narrow therapeutic index.

In any of the embodiments described herein, the substance can be an active agent having a narrow therapeutic index. Examples of narrow therapeutic index active agents include aprindine, carbamazepine, clindamycin, clonazepam, clonidine, cyclosporine, digitoxin, digoxin, disopyramide, ethinyl estradiol, ethosuximide, fosphenyloin, guanethidine, isoprenaline, lithium, methotrexate, phenobarbital, phenyloin, pimozide, prazosin, primidone, procainamide, quinidine, sulfonylurea compounds (e.g., acetohexamide, glibenclamide, gliclazide, glyclopyramide, tolazamide, tolbutamide), tacrolimus, theophylline compounds (e.g., aminophylline, choline theophylline, diprophylline, proxyphylline, and theophylline), thioridazine, valproic acid, warfarin, and zonisamide.

The invention is further illustrated by the following examples.

### Example 1

### Colchicine Inhibition of Cytochrome P450 Isozymes in Human Microsomes

The study of this example was performed to determine the potential of colchicine to inhibit the activities of cytochrome P450 isoforms CYP1A2, CYP2A6, CYP2B6, CYP2C8, CYP2C9, CYP2C19, CYP2D6, CYP2E1, and CYP3A4 in human liver microsomes. Human liver microsomes were incubated in the presence of colchicine and a substrate selective for each CYP isoform. A table of the substrate, substrate concentration, solvent, metabolite formed and metabolite assay method for each CYP isozyme studied is below.

TABLE 1
Isoform-selective substrates for cytochrome P450 isozymes.

| CYP | Isoform-selective | Substrate | | | | Metabolite |
|---|---|---|---|---|---|---|
| isoform | substrate | concentration | | Solvent | Metabolite formed | Assay |
| CYP1A2 | Phenacetin | 50 | μM | ACN | acetaminophen | LC/MS |
| CYP2A6 | Coumarin | 8 | μM | ACN | 7-hydroxycoumarin | HPLC-UV |
| CYP2B6 | S-Mephenytoin | 1 | mM | ACN | nirvanol | LC/MS |
| CYP2C8 | Paclitaxel | 5 | μM | ACN | 6-hydroxypaclitaxel | HPLC-UV |
| CYP2C9 | Tolbutamide | 150 | μM | ACN | 4′-methylhydroxytolbutamide | LC/MS |
| CYP2C19 | S-Mephenytoin | 50 | μM | ACN | 4′-hydroxymephenytoin | LC/MS |
| CYP2D6 | Dextromethorphan | 5 | μM | Water | dextrorphan | LC/MS |
| CYP2E1 | Chlorzoxazone | 50 | μM | ACN | 6-hydroxychlorzoxazone | LC/MS |
| CYP3A4 | Testosterone | 100 | μM | ACN | 6β-hydroxytestosterone | HPLC-UV |

Colchicine stock solutions were prepared in water at 100 times the final concentration and added to incubation mixtures to obtain final concentrations of 0.2, 2, 10, 20, and 50 μM, each containing 1% water and 1% acetonitrile.

Microsomes were prepared by differential centrifugation of liver homogenates pooled from at least ten human donors.

Incubation mixtures were prepared in 0.1 M Tris buffer and contained microsomes (0.25 mg protein/mL for CYP2C9, CYP2D6, CYP2E1, and CYP3A4; 0.5 mg protein/mL for CYP1A2, CYP2A6, CYP2B6, CYP2C8, and CYP2C19), colchicine, and a CYP isoform-selective substrate. All incubations were conducted at 37±1° C. in a shaking water bath. After a 5 minute preincubation, NADPH regenerating system (NRS) was added to initiate the reaction. CYP2A6 and CYP3A4 incubations were for 10 minutes. All other incubations were for 30 minutes.

Incubations for CYP2C8 were terminated by adding 1.5 mL of ACN, while all other incubations were terminated by adding 1.0 mL of methanol. Samples were transferred to cryovials and analyzed for metabolite after storage at −70° C. Three replicates were performed at each concentration of colchicine for each cytochrome P450 isozyme.

To verify that the test system was responsive to inhibitors, a positive control using ketoconazole, a selective inhibitor of CYP3A4, was added to a microsome incubation. Four replicates were performed. The test system was considered responsive to inhibitors since the mean specific activity of CYP3A4 in the positive control samples treated with ketoconazole was <22.1% of the mean specific activity in the corresponding vehicle control samples.

Vehicle control experiments were performed to establish a baseline value for enzyme activity. Incubation mixtures without added colchicine were prepared as described above. Reactions were initiated, run, and terminated as described above. Four replicates were performed.

Colchicine interference control samples were also included to eliminate the possibility of interference by colchicine or its metabolites in detection of the metabolite formed from the isoform-selective substrate. Incubation mixtures were prepared as described above containing 50 μM colchicine, but no added isoform-selective substrate. In place of the substrate, substrate solvent was added to yield a final concentration of 1%. Reactions were initiated, run, and terminated as described above. Two replicates of the interference control experiments were performed. No interference was detected in any of the metabolite assay methods used.

Results for each CYP isoform, in the presence and absence of colchicine, are reported in Tables 2-10.

TABLE 2
Colchicine Effects on CYP1A2 Activity in Pooled Human Liver Microsomes

| | Acetaminophen formation | | | | Specific Activity | | | |
|---|---|---|---|---|---|---|---|---|
| Colchicine | Raw | Adjusted (μM) | | | (pmol/min/mg protein) | | | Percent |
| (μM) | (μM) | Individual | Mean | ±SD | Individual | Mean | ±SD | of VC |
| 0 | 0.21210 | 0.212 | 0.209 | ±0.0105 | 28.3 | 27.8 | ±1.40 | 100 |
| (VC) | 0.21693 | 0.217 | | | 28.9 | | | |
| | 0.19336 | 0.193 | | | 25.8 | | | |
| | 0.21273 | 0.213 | | | 28.4 | | | |
| 0.2 | 0.18336 | 0.183 | 0.190 | ±0.00549 | 24.4 | 25.3 | ±0.732 | 90.9 |
| | 0.19283 | 0.193 | | | 25.7 | | | |
| | 0.19291 | 0.193 | | | 25.7 | | | |
| 2 | 0.20043 | 0.200 | 0.208 | ±0.00942 | 26.7 | 27.7 | ±1.26 | 99.5 |
| | 0.20457 | 0.205 | | | 27.3 | | | |
| | 0.21842 | 0.218 | | | 29.1 | | | |
| 10 | 0.21659 | 0.217 | 0.205 | ±0.0106 | 28.9 | 27.3 | ±1.42 | 98.0 |
| | 0.19632 | 0.196 | | | 26.2 | | | |
| | 0.20098 | 0.201 | | | 26.8 | | | |
| 20 | 0.18961 | 0.190 | 0.188 | ±0.00232 | 25.3 | 25.1 | ±0.309 | 90.0 |
| | 0.18534 | 0.185 | | | 24.7 | | | |
| | 0.18905 | 0.189 | | | 25.2 | | | |
| 50 | 0.19763 | 0.198 | 0.198 | ±0.00349 | 26.4 | 26.4 | ±0.465 | 94.7 |
| | 0.20139 | 0.201 | | | 26.9 | | | |
| | 0.19442 | 0.194 | | | 25.9 | | | |

Abbreviations:
SD, standard deviation;
VC, vehicle control (1% water/1% acetonitrile)
Note:
For all calculations above, the resulting values are shown with at least three significant figures for display purposes only.

TABLE 3
Colchicine Effects on CYP2A6 Activity in Pooled Human Liver Microsomes

| | 7-Hydrxoycoumarin formation | | | Specific Activity | | |
|---|---|---|---|---|---|---|
| Colchicine | Raw | Adjusted (μM) | | (pmol/min/mg protein) | | Percent |
| (μM) | (μM) | Individual | Mean ± SD | Individual | Mean ± SD | of VC |
| 0 | 0.47134 | 0.471 | 0.478 ± 0.00675 | 189 | 191 ± 2.70 | 100 |
| (VC) | 0.48746 | 0.487 | | 195 | | |
| | 0.47661 | 0.477 | | 191 | | |
| | 0.47708 | 0.477 | | 191 | | |
| 0.2 | 0.44915 | 0.449 | 0.433 ± 0.0144 | 180 | 173 ± 5.75 | 90.5 |
| | 0.42306 | 0.423 | | 169 | | |
| | 0.42562 | 0.426 | | 170 | | |
| 2 | 0.48653 | 0.487 | 0.477 ± 0.00866 | 195 | 191 ± 3.47 | 99.7 |
| | 0.47163 | 0.472 | | 189 | | |
| | 0.47142 | 0.471 | | 189 | | |
| 10 | 0.44006 | 0.440 | 0.436 ± 0.00799 | 176 | 174 ± 3.20 | 91.2 |
| | 0.44100 | 0.441 | | 176 | | |
| | 0.42671 | 0.427 | | 171 | | |
| 20 | 0.44257 | 0.443 | 0.426 ± 0.0178 | 177 | 170 ± 7.12 | 89.1 |
| | 0.42829 | 0.428 | | 171 | | |
| | 0.40719 | 0.407 | | 163 | | |
| 50 | 0.43671 | 0.437 | 0.429 ± 0.00703 | 175 | 172 ± 2.81 | 89.8 |
| | 0.42271 | 0.423 | | 169 | | |
| | 0.42865 | 0.429 | | 171 | | |

Abbreviations:
SD, standard deviation;
VC, vehicle control (1% water/1% acetonitrile)
Note:
For all calculations above, the resulting values are shown with at least three significant figures for display purposes only.

TABLE 4
Colchicine Effects on CYP2B6 Activity in Pooled Human Liver Microsomes

| | Nirvanol formation | | | Specific Activity | | |
|---|---|---|---|---|---|---|
| Colchicine | Raw | Adjusted (μM) | | (pmol/min/mg protein) | | Percent |
| (μM) | (μM) | Individual | Mean ± SD | Individual | Mean ± SD | of VC |
| 0 | 0.19120 | 0.191 | 0.185 ± 0.00593 | 25.5 | 24.7 ± 0.791 | 100 |
| (VC) | 0.18768 | 0.188 | | 25.0 | | |
| | 0.18460 | 0.185 | | 24.6 | | |
| | 0.17726 | 0.177 | | 23.6 | | |
| 0.2 | 0.14661 | 0.147 | 0.153 ± 0.00674 | 19.5 | 20.5 ± 0.899 | 82.8 |
| | 0.16009 | 0.160 | | 21.3 | | |
| | 0.15348 | 0.153 | | 20.5 | | |
| 2 | 0.19659 | 0.197 | 0.175 ± 0.0191 | 26.2 | 23.4 ± 2.54 | 94.7 |
| | 0.15951 | 0.160 | | 21.3 | | |
| | 0.17025 | 0.170 | | 22.7 | | |
| 10 | 0.16170 | 0.162 | 0.188 ± 0.0383 | 21.6 | 25.1 ± 5.10 | 102 |
| | 0.23223 | 0.232 | | 31.0 | | |
| | 0.17127 | 0.171 | | 22.8 | | |
| 20 | 0.16287 | 0.163 | 0.164 ± 0.00809 | 21.7 | 21.8 ± 1.08 | 88.4 |
| | 0.17220 | 0.172 | | 23.0 | | |
| | 0.15609 | 0.156 | | 20.8 | | |
| 50 | 0.17995 | 0.180 | 0.178 ± 0.00646 | 24.0 | 23.7 ± 0.861 | 96.0 |
| | 0.18303 | 0.183 | | 24.4 | | |
| | 0.17063 | 0.171 | | 22.8 | | |

Abbreviations:
SD, standard deviation;
VC, vehicle control (1% water/1% acetonitrile)
Note:
For all calculations above, the resulting values are shown with at least three significant figures for display purposes only.

TABLE 5
Colchicine Effects on CYP2C8 Activity in Pooled Human Liver Microsomes

| | 6-Hydroxypaclitaxel formation | | | Specific Activity | | |
|---|---|---|---|---|---|---|
| Colchicine | Raw | Adjusted (μM) | | (pmol/min/mg protein) | | Percent |
| (μM) | (μM) | Individual | Mean ± SD | Individual | Mean ± SD | of VC |
| 0 | 0.13697 | 0.137 | 0.134 ± 0.00312 | 22.8 | 22.4 ± 0.520 | 100 |
| (VC) | 0.13730 | 0.137 | | 22.9 | | |
| | 0.13127 | 0.131 | | 21.9 | | |
| | 0.13229 | 0.132 | | 22.0 | | |
| 0.2 | 0.11728 | 0.117 | 0.120 ± 0.00319 | 19.5 | 20.1 ± 0.532 | 89.5 |
| | 0.12013 | 0.120 | | 20.0 | | |
| | 0.12365 | 0.124 | | 20.6 | | |
| 2 | 0.12201 | 0.122 | 0.123 ± 0.00307 | 20.3 | 20.6 ± 0.512 | 91.8 |
| | 0.12121 | 0.121 | | 20.2 | | |
| | 0.12689 | 0.127 | | 21.1 | | |
| 10 | 0.12658 | 0.127 | 0.121 ± 0.00593 | 21.1 | 20.2 ± 0.989 | 90.3 |
| | 0.11493 | 0.115 | | 19.2 | | |
| | 0.12270 | 0.123 | | 20.5 | | |
| 20 | 0.12701 | 0.127 | 0.122 ± 0.00503 | 21.2 | 20.3 ± 0.838 | 90.8 |
| | 0.12213 | 0.122 | | 20.4 | | |
| | 0.11695 | 0.117 | | 19.5 | | |
| 50 | 0.11860 | 0.119 | 0.113 ± 0.00619 | 19.8 | 18.9 ± 1.03 | 84.3 |
| | 0.10650 | 0.107 | | 17.8 | | |
| | 0.11484 | 0.115 | | 19.1 | | |

Abbreviations:
SD, standard deviation;
VC, vehicle control (1% water/1% acetonitrile)
Note:
For all calculations above, the resulting values are shown with at least three significant figures for display purposes only.

TABLE 6
Colchicine Effects on CYP2C9 Activity in Pooled Human Liver Microsomes

| | 4′-Methylhydroxytolbutamide | | | | | |
|---|---|---|---|---|---|---|
| | formation | | | Specific Activity | | |
| Colchicine | Raw | Adjusted (μM) | | (pmol/min/mg protein) | | Percent |
| (μM) | (μM) | Individual | Mean ± SD | Individual | Mean ± SD | of VC |
| 0 | 0.23379 | 0.234 | 0.165 ± 0.0543 | 62.3 | 44.1 ± 14.5 | 100 |
| (VC) | 0.12478 | 0.125 | | 33.3 | | |
| | 0.11865 | 0.119 | | 31.6 | | |
| | 0.18400 | 0.184 | | 49.1 | | |
| 0.2 | 0.13035 | 0.130 | 0.116 ± 0.0181 | 34.8 | 30.8 ± 4.82 | 69.9 |
| | 0.12104 | 0.121 | | 32.3 | | |
| | 0.09543 | 0.0954 | | 25.4 | | |
| 2 | 0.27491 | 0.275 | 0.254 ± 0.0185 | 73.3 | 67.8 ± 4.94 | 154 |
| | 0.24949 | 0.249 | | 66.5 | | |
| | 0.23889 | 0.239 | | 63.7 | | |
| 10 | 0.15320 | 0.153 | 0.133 ± 0.0205 | 40.9 | 35.5 ± 5.46 | 80.6 |
| | 0.11226 | 0.112 | | 29.9 | | |
| | 0.13406 | 0.134 | | 35.7 | | |
| 20 | 0.17773 | 0.178 | 0.158 ± 0.0216 | 47.4 | 42.2 ± 5.75 | 95.8 |
| | 0.16209 | 0.162 | | 43.2 | | |
| | 0.13510 | 0.135 | | 36.0 | | |
| 50 | 0.16757 | 0.168 | 0.147 ± 0.0181 | 44.7 | 39.3 ± 4.82 | 89.2 |
| | 0.14235 | 0.142 | | 38.0 | | |
| | 0.13254 | 0.133 | | 35.3 | | |

Abbreviations:
SD, standard deviation;
VC, vehicle control (1% water/1% acetonitrile)
Note:
For all calculations above, the resulting values are shown with at least three significant figures for display purposes only.

TABLE 7
Colchicine Effects on CYP2C19 Activity in Pooled Human Liver Microsomes

| | 4′-Hydroxymephenytoin formation | | | Specific Activity | | |
|---|---|---|---|---|---|---|
| Colchicine | Raw | Adjusted (μM) | | (pmol/min/mg protein) | | Percent |
| (μM) | (μM) | Individual | Mean ± SD | Individual | Mean ± SD | of VC |
| 0 | 0.09628 | 0.0963 | 0.0920 ± 0.00522 | 12.8 | 12.3 ± 0.696 | 100 |
| (VC) | 0.09414 | 0.0941 | | 12.6 | | |
| | 0.09304 | 0.0930 | | 12.4 | | |
| | 0.08440 | 0.0844 | | 11.3 | | |
| 0.2 | 0.08140 | 0.0814 | 0.0840 ± 0.00266 | 10.9 | 11.2 ± 0.355 | 91.3 |
| | 0.08384 | 0.0838 | | 11.2 | | |
| | 0.08672 | 0.0867 | | 11.6 | | |
| 2 | 0.09459 | 0.0946 | 0.0986 ± 0.00359 | 12.6 | 13.1 ± 0.479 | 107 |
| | 0.10150 | 0.102 | | 13.5 | | |
| | 0.09975 | 0.0998 | | 13.3 | | |
| 10 | 0.08531 | 0.0853 | 0.0856 ± 0.00226 | 11.4 | 11.4 ± 0.301 | 93.1 |
| | 0.08351 | 0.0835 | | 11.1 | | |
| | 0.08800 | 0.0880 | | 11.7 | | |
| 20 | 0.09475 | 0.0948 | 0.0953 ± 0.000979 | 12.6 | 12.7 ± 0.131 | 104 |
| | 0.09641 | 0.0964 | | 12.9 | | |
| | 0.09468 | 0.0947 | | 12.6 | | |
| 50 | 0.09257 | 0.0926 | 0.0927 ± 0.00115 | 12.3 | 12.4 ± 0.153 | 101 |
| | 0.09156 | 0.0916 | | 12.2 | | |
| | 0.09385 | 0.0939 | | 12.5 | | |

Abbreviations:
SD, standard deviation;
VC, vehicle control (1% water/1% acetonitrile)
Note:
For all calculations above, the resulting values are shown with at least three significant figures for display purposes only.

TABLE 8
Colchicine Effects on CYP2D6 Activity in Pooled Human Liver Microsomes

| | Dextrorphan formation | | | Specific Activity | | |
|---|---|---|---|---|---|---|
| Colchicine | Raw | Adjusted (μM) | | (pmol/min/mg protein) | | Percent |
| (μM) | (μM) | Individual | Mean ± SD | Individual | Mean ± SD | of VC |
| 0 | 0.06696 | 0.0670 | 0.0616 ± 0.00836 | 17.9 | 16.4 ± 2.23 | 100 |
| (VC) | 0.06211 | 0.0621 | | 16.6 | | |
| | 0.06770 | 0.0677 | | 18.1 | | |
| | 0.04962 | 0.0496 | | 13.2 | | |
| 0.2 | 0.06445 | 0.0645 | 0.0664 ± 0.00171 | 17.2 | 17.7 ± 0.457 | 108 |
| | 0.06687 | 0.0669 | | 17.8 | | |
| | 0.06776 | 0.0678 | | 18.1 | | |
| 2 | 0.07072 | 0.0707 | 0.0691 ± 0.00296 | 18.9 | 18.4 ± 0.790 | 112 |
| | 0.07097 | 0.0710 | | 18.9 | | |
| | 0.06572 | 0.0657 | | 17.5 | | |
| 10 | 0.06348 | 0.0635 | 0.0647 ± 0.00189 | 16.9 | 17.3 ± 0.503 | 105 |
| | 0.06383 | 0.0638 | | 17.0 | | |
| | 0.06691 | 0.0669 | | 17.8 | | |
| 20 | 0.07091 | 0.0709 | 0.0733 ± 0.00230 | 18.9 | 19.5 ± 0.614 | 119 |
| | 0.07350 | 0.0735 | | 19.6 | | |
| | 0.07550 | 0.0755 | | 20.1 | | |
| 50 | 0.06545 | 0.0655 | 0.0664 ± 0.00166 | 17.5 | 17.7 ± 0.442 | 108 |
| | 0.06535 | 0.0654 | | 17.4 | | |
| | 0.06827 | 0.0683 | | 18.2 | | |

Abbreviations:
SD, standard deviation;
VC, vehicle control (1% water/1% acetonitrile)
Note:
For all calculations above, the resulting values are shown with at least three significant figures for display purposes only.

TABLE 9
Colchicine Effects on CYP2E1 Activity in Pooled Human Liver Microsomes

| | 6-Hydroxychlorzoxazone formation | | | Specific Activity | | |
|---|---|---|---|---|---|---|
| Colchicine | Raw | Adjusted (μM) | | (pmol/min/mg protein) | | Percent |
| (μM) | (μM) | Individual | Mean ± SD | Individual | Mean ± SD | of VC |
| 0 | 0.81146 | 0.811 | 0.787 ± 0.0207 | 216 | 210 ± 5.53 | 100 |
| (VC) | 0.76298 | 0.763 | | 203 | | |
| | 0.77971 | 0.780 | | 208 | | |
| | 0.79492 | 0.795 | | 212 | | |
| 0.2 | 0.76699 | 0.767 | 0.749 ± 0.0310 | 205 | 200 ± 8.27 | 95.2 |
| | 0.76713 | 0.767 | | 205 | | |
| | 0.71336 | 0.713 | | 190 | | |
| 2 | 0.81289 | 0.813 | 0.780 ± 0.0287 | 217 | 208 ± 7.65 | 99.1 |
| | 0.76807 | 0.768 | | 205 | | |
| | 0.75942 | 0.759 | | 203 | | |
| 10 | 0.72994 | 0.730 | 0.739 ± 0.00901 | 195 | 197 ± 2.40 | 93.9 |
| | 0.74788 | 0.748 | | 199 | | |
| | 0.74045 | 0.740 | | 197 | | |
| 20 | 0.79882 | 0.799 | 0.798 ± 0.0122 | 213 | 213 ± 3.26 | 101 |
| | 0.81021 | 0.810 | | 216 | | |
| | 0.78576 | 0.786 | | 210 | | |
| 50 | 0.78319 | 0.783 | 0.756 ± 0.0259 | 209 | 202 ± 6.91 | 96.0 |
| | 0.73161 | 0.732 | | 195 | | |
| | 0.75330 | 0.753 | | 201 | | |

Abbreviations:
SD, standard deviation;
VC, vehicle control (1% water/1% acetonitrile)
Note:
For all calculations above, the resulting values are shown with at least three significant figures for display purposes only.

TABLE 10
Colchicine Effects on CYP3A4 Activity in Pooled Human Liver Microsomes

| | 6β-Hydroxytestosterone formation | | | Specific Activity | | |
|---|---|---|---|---|---|---|
| Colchicine | Raw | Adjusted (μM) | | (pmol/min/mg protein) | | Percent |
| (μM) | (μM) | Individual | Mean ± SD | Individual | Mean ± SD | of VC |
| 0 | 0.43411 | 0.434 | 0.452 ± 0.0126 | 347 | 362 ± 10.1 | 100 |
| (VC) | 0.46351 | 0.464 | | 371 | | |
| | 0.45573 | 0.456 | | 365 | | |
| | 0.45533 | 0.455 | | 364 | | |
| 0.2 | 0.52049 | 0.520 | 0.539 ± 0.0162 | 416 | 431 ± 13.0 | 119 |
| | 0.55077 | 0.551 | | 441 | | |
| | 0.54570 | 0.546 | | 437 | | |
| 2 | 0.56131 | 0.561 | 0.559 ± 0.0147 | 449 | 447 ± 11.8 | 124 |
| | 0.54295 | 0.543 | | 434 | | |
| | 0.57211 | 0.572 | | 458 | | |
| 10 | 0.57041 | 0.570 | 0.543 ± 0.0262 | 456 | 435 ± 20.9 | 120 |
| | 0.51812 | 0.518 | | 414 | | |
| | 0.54193 | 0.542 | | 434 | | |
| 20 | 0.56052 | 0.561 | 0.786 ± 0.271 | 448 | 629 ± 217 | 174 |
| | 1.08621 | 1.09 | | 869 | | |
| | 0.71114 | 0.711 | | 569 | | |
| 50 | 0.74399 | 0.744 | 0.685 ± 0.0511 | 595 | 548 ± 40.9 | 151 |
| | 0.65574 | 0.656 | | 525 | | |
| | 0.65520 | 0.655 | | 524 | | |

Abbreviations:
SD, standard deviation;
VC, vehicle control (1% water/1% acetonitrile)
Note:
For all calculations above, the resulting values are shown with at least three significant figures for display purposes only.

Under these experimental conditions, no tested concentration of colchicine inhibited activity of CYP1A2 (Table 2), CYP2B6 (Table 4), CYP2C9 (Table 6), CYP2C19 (Table 7), CYP2D6 (Table 8), CYP2E1 (Table 9), or CYP3A4 (Table 10) in human liver microsomes at a statistically significant level (p>0.05 using an unpaired two-tailed t-test).

However, under these experimental conditions, colchicine did inhibit activities of CYP2A6 (Table 3) and CYP2C8 (Table 5) in human liver microsomes at one or more of the tested colchicine concentrations at a statistically significant level (p≦0.05 using an unpaired two-tailed t-test). IC₅₀ values were greater than 50 μM.

Additionally, under these experimental conditions, colchicine activated activity of CYP3A4 (Table 10) in human liver microsomes at one or more of the tested colchicine concentrations at a statistically significant level (p≦0.05 using an unpaired two-tailed t-test). The maximum activity observed was 174% of the control activity.

### Example 2

### Colchicine Induction of Cytochrome P450 Isozymes

The study of this example was performed to determine if there is induction or suppression of cytochrome P450 isozymes CYP1A2, CYP2A6, CYP2B6, CYP2C8, CYP2C9, CYP2C19, CYP2D6, CYP2E1, and CYP3A4 in human hepatocytes following in vitro exposure to colchicine. These induction/inhibition studies used cryopreserved human hepatocytes and compared enzymatic activity levels for each of these cytochrome P450 isozymes, using an appropriate enzyme substrate, in the human hepatocytes following in vitro exposure for 48±3 hrs to the presence or absence of colchicine.

Hepatocytes from three human donors were obtained from a cryopreserved hepatocyte bank (In Vitro Technologies, Inc., USA).

Donor 1 was reported to be a 40-year old Caucasian female who died of an accidental drug overdose, with a medical history including hypertension. Serology testing was negative except for cytomegalovirus. Donor 1 had a history of tobacco use (“half-pack per day for 20 years”) and drug abuse (cocaine, crack, crank, prescription drugs and marijuana). Recreational medications listed were LIBRIUM, LORTAB, and ATIVAN.

Donor 2 was reported to be a 51-year old Caucasian male who died of ischemic stroke, with a medical history including diabetes, hypertension, kidney stone removal, sleep apnea, depression/anxiety and colitis. No chronic medications were listed. Serology testing was negative except for cytomegalovirus. Donor 2 was known to smoke tobacco (“half-pack per day for 20 years”); alcohol and narcotic and cannabinoid use by Donor 2 reportedly ceased 15 years prior to donation.

Donor 3 was reported to be a 54-year old Caucasian female who died of cardiac arrest, with a medical history including high cholesterol. No chronic medications were reported Serology testing was negative, including cytomegalovirus. Donor 3 was known to smoke tobacco (“one-pack per day for 35 years”). No history of alcohol or other drug use.

After thawing, viable hepatocytes from each donor were transferred to collagen-coated 48-well plates for attachment in plating medium (DMEM stock (Dulbecco's modified Eagle's medium, supplemented with bovine serum albumin, fructose, N-(2-hydroxyethyl)piperazine-N′-(2-ethanesulfonate) (HEPES), and sodium bicarbonate), supplemented with antibiotics, bovine serum, hydrocortisone, insulin and minimum essential medium (MEM) nonessential amino acids). After attachment to the collagen matrix, plating medium was replaced with sandwich medium (incubation medium supplemented with VITROGEN and incubated until use. All incubations were conducted at 37±1° C., 95% air/5% CO2 and saturating humidity.

After establishment of the hepatocyte cultures, sandwich medium was removed and the hepatocytes were incubated with incubation solution (DMEM stock supplemented with antibiotics, hydrocortisone, insulin, and MEM non-essential amino acids) containing 0.25, 2.5, or 25 μM colchicine for 24±1.5 hrs. Incubation solution was aspirated and replaced with incubation solution containing the same concentration of colchicine and incubated for an additional 24±1.5 hrs. After the colchicine treatment period, the incubation solution was replaced with 150 μL Krebs-Henseleit (KHB) buffer supplemented with antibiotics, calcium chloride, heptanoic acid, HEPES, and sodium bicarbonate (supplemented KHB) and incubated for 10 minutes. The supplemented KHB was replaced with 150 μL supplemented KHB containing the appropriate isoform-selective substrate and incubated for 4 hrs prior to termination by adding 150 μL ice-cold methanol, except for the CYP2C8 incubations which were terminated by adding 150 μL acetonitrile. Samples were transferred to cryovials and analyzed after storage at −70° C. Three replicates were performed at each colchicine concentration for each cytochrome P450 isozyme.

Analogous vehicle control experiments were also performed to establish a baseline value for enzyme activity in the absence of colchicine. Vehicle control experiments were performed as described above for the test incubations, except that the incubation medium included no colchicine. Four replicates were performed of the vehicle control for each donor.

A table of the substrate, substrate concentration, metabolite formed, and metabolite assay method for each CYP isozyme studied is provided below. All substrates were dissolved in acetonitrile as 100× solutions. All 100× substrate solutions were diluted with supplemented KHB to the final concentrations listed below, except for paclitaxel, which was diluted with incubation medium.

TABLE 11
Isoform-selective substrates for cytochrome P450 isozymes in the colchicine
induction study.

| | Isoform-selective | Substrate | | | Metabolite |
|---|---|---|---|---|---|
| CYP isoform | substrate | concentration | | Metabolite formed | Assay |
| CYP1A2 | Phenacetin | 100 | μM | acetaminophen | LC/MS |
| CYP2A6 | Coumarin | 100 | μM | 7-hydroxycoumarin, | HPLC-UV |
| | | | | 7-hydroxy coumarin glucuronide, | |
| | | | | 7-hydroxycoumarin sulfate | |
| CYP2B6 | S-Mephenytoin | 1 | mM | nirvanol | LC/MS |
| CYP2C8 | Paclitaxel | 50 | μM | 6-hydroxy paclitaxel | HPLC-UV |
| CYP2C9 | Tolbutamide | 50 | μM | 4′-methylhydroxytolbutamide | LC/MS |
| CYP2C19 | S-Mephenytoin | 100 | μM | 4′-hydroxy mephenytoin | LC/MS |
| CYP2D6 | Dextromethorphan | 16 | μM | dextrorphan | LC/MS |
| CYP2E1 | Chlorzoxazone | 300 | μM | 6-hydroxychlorzoxazone | LC/MS |
| CYP3A4 | Testosterone | 125 | μM | 6β-hydroxy testosterone | HPLC-UV |

Colchicine 100× stock solutions were prepared in water as described above in Example 1 and diluted with incubation medium and acetonitrile to produce incubation solutions with final concentrations of 0.25, 2.5, and 25 μM colchicine, each containing 1% water and 1% acetonitrile.

Positive controls (n=4) were performed to verify that the test system was sensitive to known inducers by testing induction of CYP1A2 and CYP3A4 by 50CM omeprazole and 25 M rifampicin, respectively, using the appropriate isoform-selective enzyme substrate. Following treatment with 50 μM omeprazole, CYP1A2 activity was 653%, 765%, and 596% of the vehicle control in human hepatocytes prepared from Donors 1, 2, and 3, respectively. Following treatment with 25 μM rifampin, CYP3A4 activity was 2,796%, >2,092%, and 2,633% of the VC in human hepatocytes prepared from Donors 1, 2, and 3, respectively. Based on these increases in activities of CYP1A2 and CYP3A4 following treatment with the known inducers; the hepatocytes from the three donors were considered inducible.

Additionally, reference control samples were included to evaluate inducibility of CYP2B6, CYP2C8, CYP2C9, and CYP2C19 in the test system. The reference controls included 1 mM phenobarbital (for CYP2B6) or 25 μM rifampicin as the reference inducer. The reference controls showed a statistically significant amount of induction for each hepatocyte donor for CYP2C9, although the amount of induction varied between the three hepatocyte donors (299%, 306%, and 279% for donors 1, 2 and 3, respectively). For CYP2B6, phenobarbital-induced activity in donors 1, 2 and, 3 were 757%, 639%, and 419%, respectively. The induction for Donor 3 was calculated with the measured amounts of nirvanol formed, even thought the amount was less than the lower limit of quantitation (LLOQ) for the compound in each replicate of the vehicle control and in two of the four replicates of the rifampicin reference control. The reference controls for CYP2C8 showed a statistically significant amount of induction for each hepatocyte donor, although the amount of induction varied between the three hepatocyte donors and the measured amounts of 6-hydroxypaclitaxel formed were generally less than the lower limit of quantitation (LLOQ) for 6-hydroxypaclitaxel. For CYP2C19, rifampin induced activity in donors 1 (317%), 2 (247%) and 3 (277%). The induction of CYP2C19 by rifampicin was calculated with the measured amounts of 4′-hydroxymephenyloin formed, even though the amount was less than the lower limit of quantitation (LLOQ) for the compound in each replicate of the vehicle controls and the rifampicin reference controls. Therefore, CYP2B6, CYP2C8, CYP2C9, and CYP2C19 in the hepatocytes from these donors were considered induced by rifampin and phenobarbital.

Furthermore, interference controls were performed for each CYP isozyme to determine whether or not colchicine or its metabolites interfered with detection of the isoform-specific metabolites. In these controls, performed in duplicate, the hepatocytes were incubated with colchicine as for the test samples, and then incubated with the buffer of the isoform-specific substrate (without substrate) as for the test samples. No interference of colchicine or its metabolite was observed in any of the assays for detection of the isoform-specific metabolites formed in the test systems.

Results for each cytochrome P450 isozyme are shown in Tables 12-20. Induction was not observed at these experimental conditions for any of the tested isozymes: CYP1A2, CYP2A6, CYP2B6, CYP2C8, CYP2C9, CYP2C19, CYP2D6, CYP2E1, and CYP3A4. However, statistically significant inhibition in enzyme activity was observed for each of the nine CYPS studied. Statistical significance of a change in specific activity from that measured for the vehicle control (0 μM colchicine) was determined using a two-tailed t-test. Mean specific activity values with associated p-values≦0.05 were deemed to be statistically significant.

TABLE 12
CYP1A2 Activity in Cryopreserved Human Hepatocyte Monolayers Following
48 hr Incubation with Colchicine Prior to Substrate Addition

| | Acetaminophen formation | | | Specific Activity | | |
|---|---|---|---|---|---|---|
| Colchicine | Raw | Adjusted (μM) | | (pmol/min/million cells) | | Percent |
| (μM) | (μM) | Individual | Mean ± SD | Individual | Mean ± SD | of VC |

Human Donor 1

| | | | | | | |
|---|---|---|---|---|---|---|
| 0 | 0.18064 | 0.181 | 0.177 ± 0.00766 | 1.61 | 1.58 ± 0.0684 | 100 |
| (VC) | 0.16701 | 0.167 | | 1.49 | | |
| | 0.17518 | 0.175 | | 1.56 | | |
| | 0.18473 | 0.185 | | 1.65 | | |
| 0.25 | 0.03089 | 0.0309 | 0.0406 ± 0.0121 | 0.276 | 0.362 ± 0.108 | 22.9 |
| | 0.03671 | 0.0367 | | 0.328 | | |
| | 0.05413 | 0.0541 | | 0.483 | | |
| 2.5 | 0.04389 | 0.0439 | 0.0459 ± 0.00183 | 0.392 | 0.410 ± 0.0163 | 26.0 |
| | 0.04744 | 0.0474 | | 0.424 | | |
| | 0.04640 | 0.0464 | | 0.414 | | |
| 25 | 0.04071 | 0.0407 | 0.0415 ± 0.00248 | 0.363 | 0.371 ± 0.0222 | 23.5 |
| | 0.04433 | 0.0443 | | 0.396 | | |
| | 0.03958 | 0.0396 | | 0.353 | | |

Human Donor 2

| | | | | | | |
|---|---|---|---|---|---|---|
| 0 | 0.22463 | 0.225 | 0.236 ± 0.01034 | 2.01 | 2.11 ± 0.0923 | 100 |
| (VC) | 0.07823* | N/A | | N/A | | |
| | 0.23985 | 0.240 | | 2.14 | | |
| | 0.24437 | 0.244 | | 2.18 | | |
| 0.25 | 0.04672 | 0.0467 | 0.0462 ± 0.00145 | 0.417 | 0.413 ± 0.0130 | 19.6 |
| | 0.04732 | 0.0473 | | 0.423 | | |
| | 0.04456 | 0.0446 | | 0.398 | | |
| 2.5 | 0.04696 | 0.0470 | 0.0488 ± 0.00234 | 0.419 | 0.436 ± 0.0209 | 20.7 |
| | 0.04812 | 0.0481 | | 0.430 | | |
| | 0.05146 | 0.0515 | | 0.459 | | |
| 25 | 0.03998 | 0.0400 | 0.0423 ± 0.00207 | 0.357 | 0.378 ± 0.0185 | 17.9 |
| | 0.04382 | 0.0438 | | 0.391 | | |
| | 0.04323 | 0.0432 | | 0.386 | | |

Human Donor 3

| | | | | | | |
|---|---|---|---|---|---|---|
| 0 | 0.72070 | 0.721 | 0.757 ± 0.0439 | 6.43 | 6.76 ± 0.392 | 100 |
| (VC) | 0.71756 | 0.718 | | 6.41 | | |
| | 0.79898 | 0.799 | | 7.13 | | |
| | 0.79073 | 0.791 | | 7.06 | | |
| 0.25 | 0.15270 | 0.153 | 0.180 ± 0.0236 | 1.36 | 1.61 ± 0.211 | 23.8 |
| | 0.19188 | 0.192 | | 1.71 | | |
| | 0.19501 | 0.195 | | 1.74 | | |
| 2.5 | 0.16389 | 0.164 | 0.180 ± 0.0173 | 1.46 | 1.61 ± 0.155 | 23.8 |
| | 0.17886 | 0.179 | | 1.60 | | |
| | 0.19841 | 0.198 | | 1.77 | | |
| 25 | 0.15280 | 0.153 | 0.157 ± 0.00699 | 1.36 | 1.40 ± 0.0625 | 20.7 |
| | 0.16497 | 0.165 | | 1.47 | | |
| | 0.15291 | 0.153 | | 1.37 | | |

Abbreviations:
SD, standard deviation;
VC, vehicle control (1% water/1% acetonitrile);
N/A, not applicable
*Sample raw data value will be excluded from all calculations due to low cellular confluence observed during incubation.
Note:
For all calculations above, the resulting values are shown with at least three significant figures for display purposes only.

TABLE 13a
CYP2A6 Activity in Cryopreserved Human Hepatocyte Monolayers Following
48 hr Incubation with Colchicine Prior to Substrate Addition

| | Metabolite formation | | | Specific Activity | | |
|---|---|---|---|---|---|---|
| Colchicine | Raw | Adjusted (μM) | | (pmol/min/million cells) | | Percent |
| (μM) | (μM) | Individual | Mean ± SD | Individual | Mean ± SD | of VC |

7-Hydroxycoumarin (7-HC) Formation: Human Donor 1

| | | | | | | |
|---|---|---|---|---|---|---|
| 0 | 0.05799^{a} | <0.100 | <0.100 ± 0.000 | <0.893 | <0.893 ± 0.000 | 100 |
| (VC) | 0.05039^{a} | <0.100 | | <0.893 | | |
| | 0.06564^{a} | <0.100 | | <0.893 | | |
| | 0.03394^{a} | <0.100 | | <0.893 | | |
| 0.25 | 0.00000^{a} | <0.100 | <0.100 ± 0.000 | <0.893 | <0.893 ± 0.000 | 100 |
| | 0.00000^{a} | <0.100 | | <0.893 | | |
| | 0.00000^{a} | <0.100 | | <0.893 | | |
| 2.5 | 0.00000^{a} | <0.100 | <0.100 ± 0.000 | <0.893 | <0.893 ± 0.000 | 100 |
| | 0.00000^{a} | <0.100 | | <0.893 | | |
| | 0.00000^{a} | <0.100 | | <0.893 | | |
| 25 | 0.00000^{a} | <0.100 | <0.100 ± 0.000 | <0.893 | <0.893 ± 0.000 | 100 |
| | 0.00000^{a} | <0.100 | | <0.893 | | |
| | 0.00000^{a} | <0.100 | | <0.893 | | |

7-Hydroxycoumarin (7-HC) Formation: Human Donor 2

| | | | | | | |
|---|---|---|---|---|---|---|
| 0 | 0.00000^{a} | <0.100 | <0.100 ± 0.000 | <0.893 | <0.893 ± 0.000 | 100 |
| (VC) | 0.07417^{a} | <0.100 | | <0.893 | | |
| | 0.00000^{a} | <0.100 | | <0.893 | | |
| | 0.06269^{a} | <0.100 | | <0.893 | | |
| 0.25 | 0.06360^{a} | <0.100 | <0.100 ± 0.000 | <0.893 | <0.893 ± 0.000 | 100 |
| | 0.05338^{a} | <0.100 | | <0.893 | | |
| | 0.00000^{a} | <0.100 | | <0.893 | | |
| 2.5 | 0.00000^{a} | <0.100 | <0.100 ± 0.000 | <0.893 | <0.893 ± 0.000 | 100 |
| | 0.00000^{a} | <0.100 | | <0.893 | | |
| | 0.04979^{a} | <0.100 | | <0.893 | | |
| 25 | 0.00000^{a} | <0.100 | <0.100 ± 0.000 | <0.893 | <0.893 ± 0.000 | 100 |
| | 0.00000^{a} | <0.100 | | <0.893 | | |
| | 0.00000^{a} | <0.100 | | <0.893 | | |

7-Hydroxycoumarin (7-HC) Formation: Human Donor 3

| | | | | | | |
|---|---|---|---|---|---|---|
| 0 | 0.11042 | 0.110 | <0.104 ± 0.00501 | 0.986 | <0.928 ± 0.0447 | 100 |
| (VC) | 0.00000^{a} | <0.100 | | <0.893 | | |
| | 0.05331^{a} | <0.100 | | <0.893 | | |
| | 0.10546 | 0.105 | | 0.942 | | |
| 0.25 | 0.10795 | 0.108 | <0.103 ± 0.00459 | 0.964 | <0.917 ± 0.0410 | 98.7 |
| | 0.09518^{a} | <0.100 | | <0.893 | | |
| | 0.08086^{a} | <0.100 | | <0.893 | | |
| 2.5 | 0.08970^{a} | <0.100 | <0.100 ± 0.0000981 | <0.893 | <0.893 ± 0.000876 | 96.2 |
| | 0.10017 | 0.100 | | 0.894 | | |
| | 0.08526^{a} | <0.100 | | <0.893 | | |
| 25 | 0.19516 | 0.195 | <0.133 ± 0.0541 | 1.74 | <1.18 ± 0.483 | 128 |
| | 0.08873^{a} | <0.100 | | <0.893 | | |
| | 0.10294 | 0.103 | | 0.919 | | |

Abbreviations:
SD, standard deviation;
VC, vehicle control (1% water/1% acetonitrile)
^{a}The observed analyzed value (μM) was below the lowest concentration on the standard curve (0.1 μM).
Note:
For all calculations above, the resulting values are shown with at least three significant figures for display purposes only.

TABLE 13b
CYP2A6 Activity in Cryopreserved Human Hepatocyte Monolayers Following
48 hr Incubation with Colchicine Prior to Substrate Addition

| | Metabolite formation | | | Specific Activity | | |
|---|---|---|---|---|---|---|
| Colchicine | Raw | Adjusted (μM) | | (pmol/min/million cells) | | Percent |
| (μM) | (μM) | Individual | Mean ± SD | Individual | Mean ± SD | of VC |

7-Hydroxycoumarin Glucuronide (7-HCG) Formation: Human Donor 1

| | | | | | | |
|---|---|---|---|---|---|---|
| 0 | 1.32662 | 1.33 | 1.16 ± 0.196 | 11.8 | 10.4 ± 1.75 | 100 |
| (VC) | 1.26876 | 1.27 | | 11.3 | | |
| | 1.17175 | 1.17 | | 10.5 | | |
| | 0.88485 | 0.885 | | 7.90 | | |
| 0.25 | 0.21711 | 0.217 | 0.220 ± 0.00800 | 1.94 | 1.96 ± 0.0715 | 18.9 |
| | 0.21379 | 0.214 | | 1.91 | | |
| | 0.22901 | 0.229 | | 2.04 | | |
| 2.5 | 0.23406 | 0.234 | 0.240 ± 0.0125 | 2.09 | 2.14 ± 0.112 | 20.6 |
| | 0.23129 | 0.231 | | 2.07 | | |
| | 0.25418 | 0.254 | | 2.27 | | |
| 25 | 0.18343 | 0.183 | 0.148 ± 0.0322 | 1.64 | 1.32 ± 0.287 | 12.7 |
| | 0.13977 | 0.140 | | 1.25 | | |
| | 0.12069 | 0.121 | | 1.08 | | |

7-Hydroxycoumarin Glucuronide (7-HCG) Formation: Human Donor 2

| | | | | | | |
|---|---|---|---|---|---|---|
| 0 | 0.11407 | 0.114 | 0.102 ± 0.0121 | 1.02 | 0.908 ± 0.108 | 100 |
| (VC) | 0.10862 | 0.109 | | 0.970 | | |
| | 0.09688 | 0.0969 | | 0.865 | | |
| | 0.08699 | 0.0870 | | 0.777 | | |
| 0.25 | 0.00000^{a} | <0.0500 | <0.0500 ± 0.000 | <0.446 | <0.446 ± 0.000 | <49.2 |
| | 0.00000^{a} | <0.0500 | | <0.446 | | |
| | 0.00000^{a} | <0.0500 | | <0.446 | | |
| 2.5 | 0.01661^{a} | <0.0500 | <0.0500 ± 0.000 | <0.446 | <0.446 ± 0.000 | <49.2 |
| | 0.00000^{a} | <0.0500 | | <0.446 | | |
| | 0.00000^{a} | <0.0500 | | <0.446 | | |
| 25 | 0.00000^{a} | <0.0500 | <0.0500 ± 0.000 | <0.446 | <0.446 ± 0.000 | <49.2 |
| | 0.00000^{a} | <0.0500 | | <0.446 | | |
| | 0.00000^{a} | <0.0500 | | <0.446 | | |

7-Hydroxycoumarin Glucuronide (7-HCG) Formation: Human Donor 3

| | | | | | | |
|---|---|---|---|---|---|---|
| 0 | 1.47077 | 1.47 | 1.49 ± 0.0564 | 13.1 | 13.3 ± 0.504 | 100 |
| (VC) | 1.48203 | 1.48 | | 13.2 | | |
| | 1.42782 | 1.43 | | 12.7 | | |
| | 1.56287 | 1.56 | | 14.0 | | |
| 0.25 | 0.92785 | 0.928 | 0.722 ± 0.195 | 8.28 | 6.44 ± 1.74 | 48.6 |
| | 0.69597 | 0.696 | | 6.21 | | |
| | 0.54111 | 0.541 | | 4.83 | | |
| 2.5 | 0.67426 | 0.674 | 0.780 ± 0.101 | 6.02 | 6.97 ± 0.906 | 52.5 |
| | 0.79062 | 0.791 | | 7.06 | | |
| | 0.87632 | 0.876 | | 7.82 | | |
| 25 | 0.92590 | 0.926 | 0.772 ± 0.135 | 8.27 | 6.89 ± 1.20 | 52.0 |
| | 0.71307 | 0.713 | | 6.37 | | |
| | 0.67693 | 0.677 | | 6.04 | | |

Abbreviations:
SD, standard deviation;
VC, vehicle control (1% water/1% acetonitrile)
^{a}The observed analyzed value (μM) was below the lowest concentration on the standard curve (0.05 μM).
Note:
For all calculations above, the resulting values are shown with at least three significant figures for display purposes only.

TABLE 13c
CYP2A6 Activity in Cryopreserved Human Hepatocyte Monolayers Following
48 hr Incubation with Colchicine Prior to Substrate Addition

| | Metabolite formation | | | Specific Activity | | |
|---|---|---|---|---|---|---|
| Colchicine | Raw | Adjusted (μM) | | (pmol/min/million cells) | | Percent |
| (μM) | (μM) | Individual | Mean ± SD | Individual | Mean ± SD | of VC |

7-Hydroxycoumarin Sulfate (7-HCS) Formation: Human Donor 1

| | | | | | | |
|---|---|---|---|---|---|---|
| 0 | 0.13390^{a} | <0.150 | <0.150 ± 0.000 | <1.34 | <1.34 ± 0.000 | 100 |
| (VC) | 0.13422^{a} | <0.150 | | <1.34 | | |
| | 0.12167^{a} | <0.150 | | <1.34 | | |
| | 0.10325^{a} | <0.150 | | <1.34 | | |
| 0.25 | 0.00000^{a} | <0.150 | <0.150 ± 0.000 | <1.34 | <1.34 ± 0.000 | 100 |
| | 0.00000^{a} | <0.150 | | <1.34 | | |
| | 0.00000^{a} | <0.150 | | <1.34 | | |
| 2.5 | 0.00000^{a} | <0.150 | <0.150 ± 0.000 | <1.34 | <1.34 ± 0.000 | 100 |
| | 0.00000^{a} | <0.150 | | <1.34 | | |
| | 0.00000^{a} | <0.150 | | <1.34 | | |
| 25 | 0.00000^{a} | <0.150 | <0.150 ± 0.000 | <1.34 | <1.34 ± 0.000 | 100 |
| | 0.00000^{a} | <0.150 | | <1.34 | | |
| | 0.00000^{a} | <0.150 | | <1.34 | | |

7-Hydroxycoumarin Sulfate (7-HCS) Formation: Human Donor 2

| | | | | | | |
|---|---|---|---|---|---|---|
| 0 | 0.05679^{a} | <0.150 | <0.150 ± 0.000 | <1.34 | <1.34 ± 0.000 | 100 |
| (VC) | 0.05088^{a} | <0.150 | | <1.34 | | |
| | 0.00000^{a} | <0.150 | | <1.34 | | |
| | 0.00000^{a} | <0.150 | | <1.34 | | |
| 0.25 | 0.00000^{a} | <0.150 | <0.150 ± 0.000 | <1.34 | <1.34 ± 0.000 | 100 |
| | 0.00000^{a} | <0.150 | | <1.34 | | |
| | 0.00000^{a} | <0.150 | | <1.34 | | |
| 2.5 | 0.00000^{a} | <0.150 | <0.150 ± 0.000 | <1.34 | <1.34 ± 0.000 | 100 |
| | 0.00000^{a} | <0.150 | | <1.34 | | |
| | 0.00000^{a} | <0.150 | | <1.34 | | |
| 25 | 0.00000^{a} | <0.150 | <0.150 ± 0.000 | <1.34 | <1.34 ± 0.000 | 100 |
| | 0.00000^{a} | <0.150 | | <1.34 | | |
| | 0.00000^{a} | <0.150 | | <1.34 | | |

7-Hydroxycoumarin Sulfate (7-HCS) Formation: Human Donor 3

| | | | | | | |
|---|---|---|---|---|---|---|
| 0 | 0.00000^{a} | <0.150 | <0.150 ± 0.000 | <1.34 | <1.34 ± 0.000 | 100 |
| (VC) | 0.00000^{a} | <0.150 | | <1.34 | | |
| | 0.00000^{a} | <0.150 | | <1.34 | | |
| | 0.00000^{a} | <0.150 | | <1.34 | | |
| 0.25 | 0.00000^{a} | <0.150 | <0.150 ± 0.000 | <1.34 | <1.34 ± 0.000 | 100 |
| | 0.00000^{a} | <0.150 | | <1.34 | | |
| | 0.00000^{a} | <0.150 | | <1.34 | | |
| 2.5 | 0.00000^{a} | <0.150 | <0.150 ± 0.000 | <1.34 | <1.34 ± 0.000 | 100 |
| | 0.00000^{a} | <0.150 | | <1.34 | | |
| | 0.00000^{a} | <0.150 | | <1.34 | | |
| 25 | 0.00000^{a} | <0.150 | <0.150 ± 0.000 | <1.34 | <1.34 ± 0.000 | 100 |
| | 0.00000^{a} | <0.150 | | <1.34 | | |
| | 0.00000^{a} | <0.150 | | <1.34 | | |

Abbreviations:
SD, standard deviation;
VC, vehicle control (1% water/1% acetonitrile)
^{a}The observed analyzed value (μM) was below the lowest concentration on the standard curve (0.15 μM).
Note:
For all calculations above, the resulting values are shown with at least three significant figures for display purposes only.

TABLE 13D
CYP2A6 Activity in Cryopreserved Human Hepatocyte Monolayers Following
48 hr Incubation with Colchicine Prior to Substrate Addition

| | Metabolite formation | | | Specific Activity | | |
|---|---|---|---|---|---|---|
| Colchicine | Raw | Adjusted (μM) | | (pmol/min/million cells) | | Percent |
| (μM) | (μM) | Individual | Mean ± SD | Individual | Mean ± SD | of VC |

Total Metabolite Formation: Human Donor 1

| | | | | | | |
|---|---|---|---|---|---|---|
| 0 | 1.52^{a} | <1.58 | <1.41 ± 0.196 | <14.1 | <12.6 ± 1.75 | 100 |
| (VC) | 1.45^{a} | <1.52 | | <13.6 | | |
| | 1.36^{a} | <1.42 | | <12.7 | | |
| | 1.02^{a} | <1.13 | | <10.1 | | |
| 0.25 | 0.217^{a} | <0.467 | <0.470 ± 0.00800 | <4.17 | <4.20 ± 0.0715 | 33.3 |
| | 0.214^{a} | <0.464 | | <4.14 | | |
| | 0.229^{a} | <0.479 | | <4.28 | | |
| 2.5 | 0.234^{a} | <0.484 | <0.490 ± 0.0125 | <4.32 | <4.37 ± 0.112 | 34.7 |
| | 0.231^{a} | <0.481 | | <4.30 | | |
| | 0.254^{a} | <0.504 | | <4.50 | | |
| 25 | 0.183^{a} | <0.433 | <0.398 ± 0.0322 | <3.87 | <3.55 ± 0.287 | 28.2 |
| | 0.140^{a} | <0.390 | | <3.48 | | |
| | 0.121^{a} | <0.371 | | <3.31 | | |

Total Metabolite Formation: Human Donor 2

| | | | | | | |
|---|---|---|---|---|---|---|
| 0 | 0.171^{a} | <0.364 | <0.352 ± 0.0121 | <3.25 | <3.14 ± 0.108 | 100 |
| (VC) | 0.234^{a} | <0.359 | | <3.20 | | |
| | 0.0969^{a} | <0.347 | | <3.10 | | |
| | 0.150^{a} | <0.337 | | <3.01 | | |
| 0.25 | 0.0636^{b} | <0.300 | <0.300 ± 0.000 | <2.68 | <2.68 ± 0.000 | 85.3 |
| | 0.0534^{b} | <0.300 | | <2.68 | | |
| | 0.000^{b} | <0.300 | | <2.68 | | |
| 2.5 | 0.0166^{b} | <0.300 | <0.300 ± 0.000 | <2.68 | <2.68 ± 0.000 | 85.3 |
| | 0.000^{b} | <0.300 | | <2.68 | | |
| | 0.0498^{b} | <0.300 | | <2.68 | | |
| 25 | 0.000^{b} | <0.300 | <0.300 ± 0.000 | <2.68 | <2.68 ± 0.000 | 85.3 |
| | 0.000^{b} | <0.300 | | <2.68 | | |
| | 0.000^{b} | <0.300 | | <2.68 | | |

Total Metabolite Formation: Human Donor 3

| | | | | | | |
|---|---|---|---|---|---|---|
| 0 | 1.58^{c} | <1.73 | <1.74 ± 0.0581 | <15.5 | <15.5 ± 0.519 | 100 |
| (VC) | 1.48^{a} | <1.73 | | <15.5 | | |
| | 1.48^{a} | <1.68 | | <15.0 | | |
| | 1.67^{c} | <1.82 | | <16.2 | | |
| 0.25 | 1.04^{c} | <1.19 | <0.974 ± 0.199 | <10.6 | <8.70 ± 1.78 | 56.0 |
| | 0.791^{a} | <0.946 | | <8.45 | | |
| | 0.622^{a} | <0.791 | | <7.06 | | |
| 2.5 | 0.764^{a} | <0.924 | <1.03 ± 0.101 | <8.25 | <9.20 ± 0.906 | 59.2 |
| | 0.891^{c} | <1.04 | | <9.29 | | |
| | 0.962^{a} | <1.13 | | <10.1 | | |
| 25 | 1.12^{c} | <1.27 | <1.05 ± 0.188 | <11.3 | <9.42 ± 1.68 | 60.6 |
| | 0.802^{a} | <0.963 | | <8.60 | | |
| | 0.780^{c} | <0.930 | | <8.30 | | |

Abbreviations:
SD, standard deviation;
VC, vehicle control (1% water/1% acetonitrile)
^{a}The observed analyzed value (μM) for the 7-HC & 7-HCS metabolites were below the lowest concentration on the corresponding standard curve.
^{b}The observed analyzed value (μM) for all metabolites were below the lowest concentration on the corresponding standard curve.
^{c}The observed analyzed value (μM) for the 7-HCS metabolite was below the lowest concentration on the standard curve.
Note:
For all calculations above, the resulting values are shown with at least three significant figures for display purposes only.

TABLE 14
CYP2B6 Activity in Cryopreserved Human Hepatocyte Monolayers Following
48 hr Incubation with Colchicine Prior to Substrate Addition

| | Nirvanol formation | | | Specific Activity | | |
|---|---|---|---|---|---|---|
| Colchicine | Raw | Adjusted (μM) | | (pmol/min/million cells) | | Percent |
| (μM) | (μM) | Individual | Mean ± SD | Individual | Mean ± SD | of VC |

Human Donor 1

| | | | | | | |
|---|---|---|---|---|---|---|
| 0 | 0.07114 | 0.0711 | 0.0737 ± 0.00204 | 0.635 | 0.658 ± 0.0183 | 100 |
| (VC) | 0.07302 | 0.0730 | | 0.652 | | |
| | 0.07575 | 0.0758 | | 0.676 | | |
| | 0.07485 | 0.0749 | | 0.668 | | |
| 0.25 | 0.03628 | 0.0363 | 0.0362 ± 0.0000781 | 0.324 | 0.323 ± 0.000697 | 49.2 |
| | 0.03627 | 0.0363 | | 0.324 | | |
| | 0.03614 | 0.0361 | | 0.323 | | |
| 2.5 | 0.03089 | 0.0309 | 0.0357 ± 0.00414 | 0.276 | 0.318 ± 0.0370 | 48.4 |
| | 0.03834 | 0.0383 | | 0.342 | | |
| | 0.03776 | 0.0378 | | 0.337 | | |
| 25 | 0.03477 | 0.0348 | 0.0356 ± 0.00260 | 0.310 | 0.318 ± 0.0232 | 48.3 |
| | 0.03347 | 0.0335 | | 0.299 | | |
| | 0.03848 | 0.0385 | | 0.344 | | |

Human Donor 2

| | | | | | | |
|---|---|---|---|---|---|---|
| 0 | 0.06940 | 0.0694 | 0.0850 ± 0.0105 | 0.620 | 0.759 ± 0.0941 | 100 |
| (VC) | 0.09237 | 0.0924 | | 0.825 | | |
| | 0.08983 | 0.0898 | | 0.802 | | |
| | 0.08852 | 0.0885 | | 0.790 | | |
| 0.25 | 0.04447 | 0.0445 | 0.0420 ± 0.00424 | 0.397 | 0.375 ± 0.0378 | 49.3 |
| | 0.03706 | 0.0371 | | 0.331 | | |
| | 0.04432 | 0.0443 | | 0.396 | | |
| 2.5 | 0.04629 | 0.0463 | 0.0426 ± 0.00385 | 0.413 | 0.380 ± 0.0343 | 50.1 |
| | 0.03861 | 0.0386 | | 0.345 | | |
| | 0.04283 | 0.0428 | | 0.382 | | |
| 25 | 0.05201 | 0.0520 | 0.0510 ± 0.00314 | 0.464 | 0.455 ± 0.0281 | 60.0 |
| | 0.04747 | 0.0475 | | 0.424 | | |
| | 0.05351 | 0.0535 | | 0.478 | | |

Human Donor 3

| | | | | | | |
|---|---|---|---|---|---|---|
| 0 | 0.00601^{a} | <0.0250 | <0.0250 ± 0.000 | <0.223 | <0.223 ± 0.000 | 100 |
| (VC) | 0.00609^{a} | <0.0250 | | <0.223 | | |
| | 0.00681^{a} | <0.0250 | | <0.223 | | |
| | 0.00666^{a} | <0.0250 | | <0.223 | | |
| 0.25 | 0.00511^{a} | <0.0250 | <0.0250 ± 0.000 | <0.223 | <0.223 ± 0.000 | 100 |
| | 0.00502^{a} | <0.0250 | | <0.223 | | |
| | 0.00508^{a} | <0.0250 | | <0.223 | | |
| 2.5 | 0.00543^{a} | <0.0250 | <0.0250 ± 0.000 | <0.223 | <0.223 ± 0.000 | 100 |
| | 0.00527^{a} | <0.0250 | | <0.223 | | |
| | 0.00579^{a} | <0.0250 | | <0.223 | | |
| 25 | 0.00569^{a} | <0.0250 | <0.0250 ± 0.000 | <0.223 | <0.223 ± 0.000 | 100 |
| | 0.00600^{a} | <0.0250 | | <0.223 | | |
| | 0.00535^{a} | <0.0250 | | <0.223 | | |

Abbreviations:
SD, standard deviation;
VC, vehicle control (1% water/1% acetonitrile)
^{a}The observed analyzed value (μM) was below the lowest concentration on the standard curve (0.025 μM).
Note:
For all calculations above, the resulting values are shown with at least three significant figures for display purposes only.

TABLE 15
CYP2C8 Activity in Cryopreserved Human Hepatocyte Monolayers Following
48 hr Incubation with Colchicine Prior to Substrate Addition

| | 6-Hydroxypaclitaxel formation | | | Specific Activity | | |
|---|---|---|---|---|---|---|
| Colchicine | Raw | Adjusted (μM) | | (pmol/min/million cells) | | Percent |
| (μM) | (μM) | Individual | Mean ± SD | Individual | Mean ± SD | of VC |

Human Donor 1

| | | | | | | |
|---|---|---|---|---|---|---|
| 0 | 0.02374^{a} | <0.0500 | <0.0500 ± 0.0000 | <0.446 | <0.446 ± 0.000 | 100 |
| (VC) | 0.02292^{a} | <0.0500 | | <0.446 | | |
| | 0.01719^{a} | <0.0500 | | <0.446 | | |
| | 0.01654^{a} | <0.0500 | | <0.446 | | |
| 0.25 | 0.00815^{a} | <0.0500 | <0.0500 ± 0.0000 | <0.446 | <0.446 ± 0.000 | 100 |
| | 0.00808^{a} | <0.0500 | | <0.446 | | (39.7) |
| | 0.00769^{a} | <0.0500 | | <0.446 | | |
| 2.5 | 0.00823^{a} | <0.0500 | <0.0500 ± 0.0000 | <0.446 | <0.446 ± 0.000 | 100 |
| | 0.00802^{a} | <0.0500 | | <0.446 | | (40.5) |
| | 0.00815^{a} | <0.0500 | | <0.446 | | |
| 25 | 0.00781^{a} | <0.0500 | <0.0500 ± 0.0000 | <0.446 | <0.446 ± 0.000 | 100 |
| | 0.00748^{a} | <0.0500 | | <0.446 | | (38.8) |
| | 0.00810^{a} | <0.0500 | | <0.446 | | |

Human Donor 2

| | | | | | | |
|---|---|---|---|---|---|---|
| 0 | 0.01378^{a} | <0.0500 | <0.0500 ± 0.0000 | <0.446 | <0.446 ± 0.000 | 100 |
| (VC) | 0.01243^{a} | <0.0500 | | <0.446 | | |
| | 0.01247^{a} | <0.0500 | | <0.446 | | |
| | 0.01208^{a} | <0.0500 | | <0.446 | | |
| 0.25 | 0.00000^{a} | <0.0500 | <0.0500 ± 0.0000 | <0.446 | <0.446 ± 0.000 | 100 |
| | 0.00000^{a} | <0.0500 | | <0.446 | | |
| | 0.00000^{a} | <0.0500 | | <0.446 | | |
| 2.5 | 0.00000^{a} | <0.0500 | <0.0500 ± 0.0000 | <0.446 | <0.446 ± 0.000 | 100 |
| | 0.00000^{a} | <0.0500 | | <0.446 | | |
| | 0.00000^{a} | <0.0500 | | <0.446 | | |
| 25 | 0.00000^{a} | <0.0500 | <0.0500 ± 0.0000 | <0.446 | <0.446 ± 0.000 | 100 |
| | 0.00000^{a} | <0.0500 | | <0.446 | | |
| | 0.00000^{a} | <0.0500 | | <0.446 | | |

Human Donor 3

| | | | | | | |
|---|---|---|---|---|---|---|
| 0 | 0.02195^{a} | <0.0500 | <0.0500 ± 0.0000 | <0.446 | <0.446 ± 0.000 | 100 |
| (VC) | 0.02203^{a} | <0.0500 | | <0.446 | | |
| | 0.02217^{a} | <0.0500 | | <0.446 | | |
| | 0.02098^{a} | <0.0500 | | <0.446 | | |
| 0.25 | 0.00899^{a} | <0.0500 | <0.0500 ± 0.0000 | <0.446 | <0.446 ± 0.000 | 100 |
| | 0.00910^{a} | <0.0500 | | <0.446 | | (41.3) |
| | 0.00887^{a} | <0.0500 | | <0.446 | | |
| 2.5 | 0.00913^{a} | <0.0500 | <0.0500 ± 0.0000 | <0.446 | <0.446 ± 0.000 | 100 |
| | 0.00924^{a} | <0.0500 | | <0.446 | | (41.9) |
| | 0.00901^{a} | <0.0500 | | <0.446 | | |
| 25 | 0.00869^{a} | <0.0500 | <0.0500 ± 0.0000 | <0.446 | <0.446 ± 0.000 | 100 |
| | 0.00864^{a} | <0.0500 | | <0.446 | | (39.7) |
| | 0.00863^{a} | <0.0500 | | <0.446 | | |

Abbreviations:
SD, standard deviation;
VC, vehicle control (1% water/1% acetonitrile)
^{a}The observed analyzed value (μM) was below the lowest concentration on the standard curve (0.05 μM).
Note:
For all calculations above, the resulting values are shown with at least three significant figures for display purposes only.

TABLE 16
CYP2C9 Activity in Cryopreserved Human Hepatocyte Monolayers Following
48 hr Incubation with Colchicine Prior to Substrate Addition

| | 4′-Methylhydroxytolbutamide formation | | | Specific Activity | | |
|---|---|---|---|---|---|---|
| Colchicine | Raw | Adjusted (μM) | | (pmol/min/million cells) | | Percent |
| (μM) | (μM) | Individual | Mean ± SD | Individual | Mean ± SD | of VC |

Human Donor 1

| | | | | | | |
|---|---|---|---|---|---|---|
| 0 | 0.13088 | 0.131 | 0.157 ± 0.0176 | 1.17 | 1.40 ± 0.157 | 100 |
| (VC) | 0.16799 | 0.168 | | 1.50 | | |
| | 0.16772 | 0.168 | | 1.50 | | |
| | 0.16085 | 0.161 | | 1.44 | | |
| 0.25 | 0.06733 | 0.0673 | 0.0813 ± 0.0137 | 0.601 | 0.726 ± 0.122 | 51.8 |
| | 0.08193 | 0.0819 | | 0.732 | | |
| | 0.09465 | 0.0947 | | 0.845 | | |
| 2.5 | 0.05931 | 0.0593 | 0.0718 ± 0.0110 | 0.530 | 0.641 ± 0.0983 | 45.8 |
| | 0.07618 | 0.0762 | | 0.680 | | |
| | 0.08000 | 0.0800 | | 0.714 | | |
| 25 | 0.06804 | 0.0680 | 0.0723 ± 0.00488 | 0.608 | 0.645 ± 0.0436 | 46.1 |
| | 0.07118 | 0.0712 | | 0.636 | | |
| | 0.07762 | 0.0776 | | 0.693 | | |

Human Donor 2

| | | | | | | |
|---|---|---|---|---|---|---|
| 0 | 0.02947 | 0.0295 | 0.0342 ± 0.00673 | 0.263 | 0.305 ± 0.0601 | 100 |
| (VC) | N/A* | N/A* | | N/A* | | |
| | 0.03112 | 0.0311 | | 0.278 | | |
| | 0.04186 | 0.0419 | | 0.374 | | |
| 0.25 | 0.01570 | 0.0157 | 0.0138 ± 0.00170 | 0.140 | 0.123 ± 0.0152 | 40.4 |
| | 0.01323 | 0.0132 | | 0.118 | | |
| | 0.01243 | 0.0124 | | 0.111 | | |
| 2.5 | 0.01871 | 0.0187 | 0.0191 ± 0.00329 | 0.167 | 0.171 ± 0.0293 | 55.9 |
| | 0.01602 | 0.0160 | | 0.143 | | |
| | 0.02256 | 0.0226 | | 0.201 | | |
| 25 | 0.01745 | 0.0175 | 0.0163 ± 0.00102 | 0.156 | 0.145 ± 0.00906 | 47.7 |
| | 0.01558 | 0.0156 | | 0.139 | | |
| | 0.01583 | 0.0158 | | 0.141 | | |

Human Donor 3

| | | | | | | |
|---|---|---|---|---|---|---|
| 0 | 0.15807 | 0.158 | 0.162 ± 0.0228 | 1.41 | 1.45 ± 0.204 | 100 |
| (VC) | 0.14713 | 0.147 | | 1.31 | | |
| | 0.19520 | 0.195 | | 1.74 | | |
| | 0.14706 | 0.147 | | 1.31 | | |
| 0.25 | 0.08707 | 0.0871 | 0.0871 ± 0.00248 | 0.777 | 0.778 ± 0.0221 | 53.8 |
| | 0.08966 | 0.0897 | | 0.801 | | |
| | 0.08471 | 0.0847 | | 0.756 | | |
| 2.5 | 0.08908 | 0.0891 | 0.0886 ± 0.00565 | 0.795 | 0.791 ± 0.0504 | 54.7 |
| | 0.08271 | 0.0827 | | 0.738 | | |
| | 0.09397 | 0.0940 | | 0.839 | | |
| 25 | 0.08904 | 0.0890 | 0.0947 ± 0.00816 | 0.795 | 0.846 ± 0.0729 | 58.5 |
| | 0.10407 | 0.104 | | 0.929 | | |
| | 0.09104 | 0.0910 | | 0.813 | | |

Abbreviations:
SD, standard deviation;
VC, vehicle control (1% water/1% acetonitrile);
N/A, not applicable
*Sample was unavailable for analysis due to autosampler error.
Note:
For all calculations above, the resulting values are shown with at least three significant figures for display purposes only.

TABLE 17
CYP2C19 Activity in Cryopreserved Human Hepatocyte Monolayers Following
48 hr Incubation with Colchicine Prior to Substrate Addition

| | 4′-Hydroxymephenytoin formation | | | Specific Activity | | |
|---|---|---|---|---|---|---|
| Colchicine | Raw | Adjusted (μM) | | (pmol/min/million cells) | | Percent |
| (μM) | (μM) | Individual | Mean ± SD | Individual | Mean ± SD | of VC |

Human Donor 1

| | | | | | | |
|---|---|---|---|---|---|---|
| 0 | 0.00757^{a} | <0.0500 | <0.0500 ± 0.0000 | <0.446 | <0.446 ± 0.000 | 100 |
| (VC) | 0.00687^{a} | <0.0500 | | <0.446 | | |
| | 0.00763^{a} | <0.0500 | | <0.446 | | |
| | 0.00755^{a} | <0.0500 | | <0.446 | | |
| 0.25 | 0.00555^{a} | <0.0500 | <0.0500 ± 0.0000 | <0.446 | <0.446 ± 0.000 | 100 |
| | 0.00523^{a} | <0.0500 | | <0.446 | | (71.7%) |
| | 0.00516^{a} | <0.0500 | | <0.446 | | |
| 2.5 | 0.00491^{a} | <0.0500 | <0.0500 ± 0.0000 | <0.446 | <0.446 ± 0.000 | 100 |
| | 0.00524^{a} | <0.0500 | | <0.446 | | (69.2%) |
| | 0.00522^{a} | <0.0500 | | <0.446 | | |
| 25 | 0.00457^{a} | <0.0500 | <0.0500 ± 0.0000 | <0.446 | <0.446 ± 0.000 | 100 |
| | 0.00461^{a} | <0.0500 | | <0.446 | | (62.3%) |
| | 0.00466^{a} | <0.0500 | | <0.446 | | |

Human Donor 2

| | | | | | | |
|---|---|---|---|---|---|---|
| 0 | 0.00871^{a} | <0.0500 | <0.0500 ± 0.0000 | <0.446 | <0.446 ± 0.000 | 100 |
| (VC) | 0.00877^{a} | <0.0500 | | <0.446 | | |
| | 0.00826^{a} | <0.0500 | | <0.446 | | |
| | 0.00922^{a} | <0.0500 | | <0.446 | | |
| 0.25 | 0.00726^{a} | <0.0500 | <0.0500 ± 0.0000 | <0.446 | <0.446 ± 0.000 | 100 |
| | 0.00768^{a} | <0.0500 | | <0.446 | | (82.5%) |
| | 0.00670^{a} | <0.0500 | | <0.446 | | |
| 2.5 | 0.00707^{a} | <0.0500 | <0.0500 ± 0.0000 | <0.446 | <0.446 ± 0.000 | 100 |
| | 0.00649^{a} | <0.0500 | | <0.446 | | (79.8%) |
| | 0.00736^{a} | <0.0500 | | <0.446 | | |
| 25 | 0.00704^{a} | <0.0500 | <0.0500 ± 0.0000 | <0.446 | <0.446 ± 0.000 | 100 |
| | 0.00612^{a} | <0.0500 | | <0.446 | | (74.5%) |
| | 0.00636^{a} | <0.0500 | | <0.446 | | |

Human Donor 3

| | | | | | | |
|---|---|---|---|---|---|---|
| 0 | 0.01106^{a} | <0.0500 | <0.0500 ± 0.0000 | <0.446 | <0.446 ± 0.000 | 100 |
| (VC) | 0.01082^{a} | <0.0500 | | <0.446 | | |
| | 0.01317^{a} | <0.0500 | | <0.446 | | |
| | 0.01098^{a} | <0.0500 | | <0.446 | | |
| 0.25 | 0.00771^{a} | <0.0500 | <0.0500 ± 0.0000 | <0.446 | <0.446 ± 0.000 | 100 |
| | 0.00668^{a} | <0.0500 | | <0.446 | | (64.7%) |
| | 0.00795^{a} | <0.0500 | | <0.446 | | |
| 2.5 | 0.00844^{a} | <0.0500 | <0.0500 ± 0.0000 | <0.446 | <0.446 ± 0.000 | 100 |
| | 0.00808^{a} | <0.0500 | | <0.446 | | (70.6%) |
| | 0.00786^{a} | <0.0500 | | <0.446 | | |
| 25 | 0.00666^{a} | <0.0500 | <0.0500 ± 0.0000 | <0.446 | <0.446 ± 0.000 | 100 |
| | 0.00846^{a} | <0.0500 | | <0.446 | | (65.3%) |
| | 0.00744^{a} | <0.0500 | | <0.446 | | |

Abbreviations:
SD, standard deviation;
VC, vehicle control (1% water/1% acetonitrile)
^{a}The observed analyzed value (μM) was below the lowest concentration on the standard curve (0.05 μM).
Note:
For all calculations above, the resulting values are shown with at least three significant figures for display purposes only.

TABLE 18
CYP2D6 Activity in Cryopreserved Human Hepatocyte Monolayers Following
48 hr Incubation with Colchicine Prior to Substrate Addition

| | Dextrorphan formation | | | Specific Activity | | |
|---|---|---|---|---|---|---|
| Colchicine | Raw | Adjusted (μM) | | (pmol/min/million cells) | | Percent |
| (μM) | (μM) | Individual | Mean ± SD | Individual | Mean ± SD | of VC |

Human Donor 1

| | | | | | | |
|---|---|---|---|---|---|---|
| 0 | 0.06354 | 0.0635 | 0.0652 ± 0.00181 | 0.567 | 0.582 ± 0.0161 | 100 |
| (VC) | 0.06416 | 0.0642 | | 0.573 | | |
| | 0.06560 | 0.0656 | | 0.586 | | |
| | 0.06761 | 0.0676 | | 0.604 | | |
| 0.25 | 0.02619 | 0.0262 | 0.0285 ± 0.00204 | 0.234 | 0.254 ± 0.0182 | 43.6 |
| | 0.02910 | 0.0291 | | 0.260 | | |
| | 0.03012 | 0.0301 | | 0.269 | | |
| 2.5 | 0.02623 | 0.0262 | 0.0286 ± 0.00203 | 0.234 | 0.255 ± 0.0181 | 43.8 |
| | 0.02949 | 0.0295 | | 0.263 | | |
| | 0.02995 | 0.0300 | | 0.267 | | |
| 25 | 0.02551 | 0.0255 | 0.0288 ± 0.00291 | 0.228 | 0.258 ± 0.0260 | 44.2 |
| | 0.03019 | 0.0302 | | 0.270 | | |
| | 0.03084 | 0.0308 | | 0.275 | | |

Human Donor 2

| | | | | | | |
|---|---|---|---|---|---|---|
| 0 | 0.00900^{a} | <0.0100 | <0.0103 ± 0.000620 | <0.0893 | <0.0921 ± 0.00554 | 100 |
| (VC) | 0.00943^{a} | <0.0100 | | <0.0893 | | |
| | 0.01124 | 0.0112 | | 0.100 | | |
| | 0.00959^{a} | <0.0100 | | <0.0893 | | |
| 0.25 | 0.00000^{a} | <0.0100 | <0.0100 ± 0.000 | <0.0893 | <0.0893 ± 0.000 | 97.0 |
| | 0.00000^{a} | <0.0100 | | <0.0893 | | |
| | 0.00000^{a} | <0.0100 | | <0.0893 | | |
| 2.5 | 0.00000^{a} | <0.0100 | <0.0100 ± 0.000 | <0.0893 | <0.0893 ± 0.000 | 97.0 |
| | 0.00000^{a} | <0.0100 | | <0.0893 | | |
| | 0.00000^{a} | <0.0100 | | <0.0893 | | |
| 25 | 0.00000^{a} | <0.0100 | <0.0100 ± 0.000 | <0.0893 | <0.0893 ± 0.000 | 97.0 |
| | 0.00000^{a} | <0.0100 | | <0.0893 | | |
| | 0.00000^{a} | <0.0100 | | <0.0893 | | |

Human Donor 3

| | | | | | | |
|---|---|---|---|---|---|---|
| 0 | 0.16083 | 0.161 | 0.161 ± 0.00194 | 1.44 | 1.44 ± 0.0173 | 100 |
| (VC) | 0.16420 | 0.164 | | 1.47 | | |
| | 0.15972 | 0.160 | | 1.43 | | |
| | 0.16086 | 0.161 | | 1.44 | | |
| 0.25 | 0.08660 | 0.0866 | 0.0842 ± 0.00220 | 0.773 | 0.752 ± 0.0196 | 52.2 |
| | 0.08228 | 0.0823 | | 0.735 | | |
| | 0.08371 | 0.0837 | | 0.747 | | |
| 2.5 | 0.08375 | 0.0838 | 0.0849 ± 0.00101 | 0.748 | 0.758 ± 0.00904 | 52.6 |
| | 0.08517 | 0.0852 | | 0.760 | | |
| | 0.08571 | 0.0857 | | 0.765 | | |
| 25 | 0.09870 | 0.0987 | 0.0932 ± 0.00519 | 0.881 | 0.832 ± 0.0464 | 57.8 |
| | 0.08837 | 0.0884 | | 0.789 | | |
| | 0.09259 | 0.0926 | | 0.827 | | |

Abbreviations:
SD, standard deviation;
VC, vehicle control (1% water/1% acetonitrile)
^{a}The observed analyzed value (μM) was below the lowest concentration on the standard curve (0.01 μM).
Note:
For all calculations above, the resulting values are shown with at least three significant figures for display purposes only.

TABLE 19
CYP2E1 Activity in Cryopreserved Human Hepatocyte Monolayers Following
48 hr Incubation with Colchicine Prior to Substrate Addition

| | 6-Hydroxychlorzoxazone formation | | | Specific Activity | | |
|---|---|---|---|---|---|---|
| Colchicine | Raw | Adjusted (μM) | | (pmol/min/million cells) | | Percent |
| (μM) | (μM) | Individual | Mean ± SD | Individual | Mean ± SD | of VC |

Human Donor 1

| | | | | | | |
|---|---|---|---|---|---|---|
| 0 | 0.15265 | 0.153 | 0.163 ± 0.0106 | 1.36 | 1.45 ± 0.0949 | 100 |
| (VC) | 0.15873 | 0.159 | | 1.42 | | |
| | 0.16111 | 0.161 | | 1.44 | | |
| | 0.17752 | 0.178 | | 1.59 | | |
| 0.25 | 0.10828 | 0.108 | 0.109 ± 0.00240 | 0.967 | 0.977 ± 0.0214 | 67.4 |
| | 0.11224 | 0.112 | | 1.00 | | |
| | 0.10791 | 0.108 | | 0.963 | | |
| 2.5 | 0.11581 | 0.116 | 0.113 ± 0.00232 | 1.03 | 1.01 ± 0.0207 | 69.7 |
| | 0.11131 | 0.111 | | 0.994 | | |
| | 0.11261 | 0.113 | | 1.01 | | |
| 25 | 0.11066 | 0.111 | 0.112 ± 0.00738 | 0.988 | 0.997 ± 0.0659 | 68.7 |
| | 0.10490 | 0.105 | | 0.937 | | |
| | 0.11955 | 0.120 | | 1.07 | | |

Human Donor 2

| | | | | | | |
|---|---|---|---|---|---|---|
| 0 | 0.07977 | 0.0798 | 0.0806 ± 0.00616 | 0.712 | 0.720 ± 0.0550 | 100 |
| (VC) | 0.08298 | 0.0830 | | 0.741 | | |
| | 0.07255 | 0.0726 | | 0.648 | | |
| | 0.08712 | 0.0871 | | 0.778 | | |
| 0.25 | 0.04351 | 0.0435 | 0.0500 ± 0.00580 | 0.388 | 0.447 ± 0.0518 | 62.1 |
| | 0.05192 | 0.0519 | | 0.464 | | |
| | 0.05464 | 0.0546 | | 0.488 | | |
| 2.5 | 0.06000 | 0.0600 | 0.0588 ± 0.00184 | 0.536 | 0.525 ± 0.0164 | 72.9 |
| | 0.05666 | 0.0567 | | 0.506 | | |
| | 0.05965 | 0.0597 | | 0.533 | | |
| 25 | 0.05587 | 0.0559 | 0.0566 ± 0.000955 | 0.499 | 0.505 ± 0.00853 | 70.2 |
| | 0.05616 | 0.0562 | | 0.501 | | |
| | 0.05765 | 0.0577 | | 0.515 | | |

Human Donor 3

| | | | | | | |
|---|---|---|---|---|---|---|
| 0 | 0.04012 | 0.0401 | 0.0393 ± 0.00443 | 0.358 | 0.351 ± 0.0396 | 100 |
| (VC) | 0.04016 | 0.0402 | | 0.359 | | |
| | 0.03314 | 0.0331 | | 0.296 | | |
| | 0.04373 | 0.0437 | | 0.390 | | |
| 0.25 | 0.02186 | 0.0219 | 0.0177 ± 0.00512 | 0.195 | 0.158 ± 0.0457 | 45.1 |
| | 0.01931 | 0.0193 | | 0.172 | | |
| | 0.01199 | 0.0120 | | 0.107 | | |
| 2.5 | 0.01799 | 0.0180 | 0.0175 ± 0.000956 | 0.161 | 0.156 ± 0.00853 | 44.4 |
| | 0.01635 | 0.0164 | | 0.146 | | |
| | 0.01802 | 0.0180 | | 0.161 | | |
| 25 | 0.01562 | 0.0156 | 0.0187 ± 0.00311 | 0.139 | 0.167 ± 0.0277 | 47.7 |
| | 0.02183 | 0.0218 | | 0.195 | | |
| | 0.01872 | 0.0187 | | 0.167 | | |

Abbreviations:
SD, standard deviation;
VC, vehicle control (1% water/1% acetonitrile)
Note:
For all calculations above, the resulting values are shown with at least three significant figures for display purposes only.

TABLE 20
CYP3A4 Activity in Cryopreserved Human Hepatocyte Monolayers Following
48 hr Incubation with Colchicine Prior to Substrate Addition

| | 6β-Hydroxytestosterone formation | | | Specific Activity | | |
|---|---|---|---|---|---|---|
| Colchicine | Raw | Adjusted (μM) | | (pmol/min/million cells) | | Percent |
| (μM) | (μM) | Individual | Mean ± SD | Individual | Mean ± SD | of VC |

Human Donor 1

| | | | | | | |
|---|---|---|---|---|---|---|
| 0 | 0.41879 | 0.419 | 0.379 ± 0.0415 | 3.74 | 3.38 ± 0.370 | 100 |
| (VC) | 0.37573 | 0.376 | | 3.35 | | |
| | 0.39822 | 0.398 | | 3.56 | | |
| | 0.32251 | 0.323 | | 2.88 | | |
| 0.25 | 0.11124 | 0.111 | 0.166 ± 0.0510 | 0.993 | 1.48 ± 0.456 | 43.8 |
| | 0.21218 | 0.212 | | 1.89 | | |
| | 0.17479 | 0.175 | | 1.56 | | |
| 2.5 | 0.14595 | 0.146 | 0.152 ± 0.00674 | 1.30 | 1.36 ± 0.0602 | 40.2 |
| | 0.15160 | 0.152 | | 1.35 | | |
| | 0.15937 | 0.159 | | 1.42 | | |
| 25* | 0.00000^{a} | <0.100 | <0.100 ± 0.000 | <0.893 | <0.893 ± 0.000 | 26.4 |
| | 0.00000^{a} | <0.100 | | <0.893 | | |
| | 0.00000^{a} | <0.100 | | <0.893 | | |

Human Donor 2

| | | | | | | |
|---|---|---|---|---|---|---|
| 0 | 0.04579^{a} | <0.100 | <0.125 ± 0.0172 | <0.893 | <1.12 ± 0.153 | 100 |
| (VC) | 0.13621 | 0.136 | | 1.22 | | |
| | 0.13055 | 0.131 | | 1.17 | | |
| | 0.13513 | 0.135 | | 1.21 | | |
| 0.25 | 0.04686^{a} | <0.100 | <0.100 ± 0.000 | <0.893 | <0.893 ± 0.000 | 79.7 |
| | 0.08440^{a} | <0.100 | | <0.893 | | |
| | 0.08079^{a} | <0.100 | | <0.893 | | |
| 2.5 | 0.06037^{a} | <0.100 | <0.100 ± 0.000 | <0.893 | <0.893 ± 0.000 | 79.7 |
| | 0.06539^{a} | <0.100 | | <0.893 | | |
| | 0.06427^{a} | <0.100 | | <0.893 | | |
| 25* | 0.00000^{a} | <0.100 | <0.100 ± 0.000 | <0.893 | <0.893 ± 0.000 | 79.7 |
| | 0.00000^{a} | <0.100 | | <0.893 | | |
| | 0.00000^{a} | <0.100 | | <0.893 | | |

Human Donor 3

| | | | | | | |
|---|---|---|---|---|---|---|
| 0 | 0.50361 | 0.504 | 0.518 ± 0.0127 | 4.50 | 4.63 ± 0.113 | 100 |
| (VC) | 0.53433 | 0.534 | | 4.77 | | |
| | 0.51554 | 0.516 | | 4.60 | | |
| | 0.51948 | 0.519 | | 4.64 | | |
| 0.25 | 0.22105 | 0.221 | 0.201 ± 0.0380 | 1.97 | 1.80 ± 0.339 | 38.9 |
| | 0.22571 | 0.226 | | 2.02 | | |
| | 0.15772 | 0.158 | | 1.41 | | |
| 2.5 | 0.24948 | 0.249 | 0.244 ± 0.00500 | 2.23 | 2.18 ± 0.0446 | 47.0 |
| | 0.24106 | 0.241 | | 2.15 | | |
| | 0.24061 | 0.241 | | 2.15 | | |
| 25* | 0.00000^{a} | <0.100 | <0.118 ± 0.0157 | <0.893 | <1.05 ± 0.141 | <22.7 |
| | 0.13019 | 0.130 | | 1.16 | | |
| | 0.12285 | 0.123 | | 1.10 | | |

Abbreviations:
SD, standard deviation;
VC, vehicle control (1% water/1% acetonitrile)
*Test Article interference was observed in each of these samples.
^{a}The observed analyzed value (μM) was below the lowest concentration on the standard curve (0.1 μM).
Note:
For all calculations above, the resulting values are shown with at least three significant figures for display purposes only.

CYP1A2 activity in cryopreserved human hepatocytes was quantified by measuring the formation of acetaminophen from phenacetin. Following treatment with 50 μM omeprazole, a known inducer for CYP1A2, CYP1A2 activity was 653%, 765%, and 596% of the vehicle control (VC, 1% acetonitrile with 1% water) in human hepatocytes prepared from Donors 1, 2, and 3, respectively. CYP3A4 activity in cryopreserved human hepatocytes was quantified by measuring the formation of 6b hydroxytestosterone from testosterone. Following treatment with 25 μM rifampin, a known inducer for CYP3A4, CYP3A4 activity was 2,796%, >2,092%, and 2,633% of the VC in human hepatocytes prepared from Donors 1, 2, and 3, respectively. The increase in activities of CYP1A2 and CYP3A4 following treatment with the known inducers indicate that the hepatocytes from these donors were inducible.

Colchicine at the tested concentrations did not induce CYP1A2 activity in human hepatocytes prepared from all three donors, instead a significant suppression of enzyme activity was observed. This conclusion was based on CYP1A2 activity (22.9, 26.0, and 23.5% of the VC in Donor 1; 19.6, 20.7, and 17.9% of the VC in Donor 2; and 23.8, 23.8, and 20.7% of the VC in Donor 3) in hepatocytes treated with 0.25, 2.5, and 25 μM colchicine (Table 12). The assay method detected no chromatographic interference from colchicine or its metabolite.

CYP2A6 activity in cryopreserved human hepatocytes was quantified by adding coumarin to the hepatocytes and measuring the formation of 7-hydroxycoumarin (7-HC) and its conjugated derivatives, 7-hydroxycoumarin glucuronide (7-HCG) and 7-hydroxycoumarin sulfate (7-HCS). Colchicine at the tested concentrations did not induce CYP2A6 activity in human hepatocytes prepared from all three donors, instead a significant suppression of enzyme activity was observed. This conclusion was based on the amount of 7-HC, 7-HCG, 7-HCS, or the sum of the above three metabolites formed in hepatocytes treated with 0.25, 2.5, and 25 μM colchicine (Tables 13a-13d). The assay method detected no chromatographic interference from colchicine or its metabolite.

CYP2B6 activity in cryopreserved human hepatocytes was quantified by adding S-mephenyloin to the hepatocytes and measuring the formation of its metabolite, nirvanol. Colchicine at the tested concentrations did not induce CYP2B6 activity in human hepatocytes prepared from all three donors, instead significant suppression of enzyme activity was observed. This conclusion was based on CYP2B6 activity (49.2, 48.4, and 48.3% of the VC in Donor 1; 49.3, 50.1, and 60.0% of the VC in Donor 2; and 100, 100, and 100% of the VC in Donor 3) in hepatocytes treated with 0.25, 2.5, and 25 μM colchicine (Table 14). The assay method detected no chromatographic interference from colchicine or its metabolite.

CYP2C8 activity in cryopreserved human hepatocytes was quantified by adding paclitaxel to the hepatocytes and measuring the formation of its metabolite, 6-hydropaclitaxel. Colchicine at the tested concentrations did not induce CYP2C8 activity in human hepatocytes isolated from all three donors, instead significant suppression of enzyme activity was observed. This conclusion was based on CYP2C8 activity in hepatocytes treated with 0.25, 2.5, and 25 μM colchicine (Table 15) calculated for Donors 1 and 3 using the measured amounts of 6-hydroxypaclitaxel formed, even though these amounts were generally less than the LLOQ for 6-hydroxypaclitaxel, and on the observation that for Donor 2 that the measured amount of 6-hydroxypaclitaxel for the VC was lowered to an undetectable amount in each of the samples with colchicine. The assay method detected no chromatographic interference from colchicine or its metabolite.

CYP2C9 activity in cryopreserved human hepatocytes was quantified by adding tolbutamide to the hepatocytes and measuring the formation of its metabolite, 4′-methylhydroxytolbutamide. Colchicine at the tested concentrations did not induce CYP2C9 activity in human hepatocytes prepared from all three donors, but did significantly suppress CYP2C9 enzyme activity. This conclusion was based on CYP2C9 activity (51.8, 45.8, and 46.1% of the VC in Donor 1; 40.4, 55.9, and 47.7% of the VC in Donor 2; and 53.8, 54.7, and 58.5% of the VC in Donor 3) in hepatocytes treated with 0.25, 2.5, and 25 μM colchicine (Table 16). The assay method detected no chromatographic interference from colchicine or its metabolite.

CYP2C19 activity in cryopreserved human hepatocytes was quantified by adding S-mephenyloin to the hepatocytes and measuring the formation of its metabolite, 4′-hydroxymephenyloin. Levels of 4′-hydroxymephenyloin from CYP2C19 activity in hepatocytes treated with vehicle or 0.25, 2.5 and 25 mM of colchicine was below the LLOQ (Table 17), however when these measured values were used colchicine at the concentrations tested did not induce CYP2C19 activity in human hepatocytes isolated from all three donors. Instead statistically significant suppression of CYP2C19 enzyme activity was observed in all three donors. The assay method detected no chromatographic interference from colchicine or its metabolite.

CYP2D6 activity in cryopreserved human hepatocytes was quantified by adding dextromethorphan to the hepatocytes and measuring the formation of its metabolite, dextrorphan. Colchicine at the concentrations tested did not induce CYP2D6 activity in human hepatocytes isolated from all three donors, but it did suppress CYP2D6 activity at a statistically significant level. This conclusion was based on CYP2D6 activity (43.6, 43.8, and 44.2% of the VC in Donor 1; and 52.2, 52.6, and 57.8% of the VC in Donor 3) in hepatocytes treated with 0.25, 2.5, and 25 μM colchicine (Table 18). For Donor 2, the VC samples were below the LLOQ for dextrorphan, however for the samples of each of the three colchicine concentrations tested, no dextrorphan was measurable in the samples, an observation qualitatively consistent with suppression of CYP2D6 activity in Donor 2 as well. The assay method detected no chromatographic interference from colchicine or its metabolite.

CYP2E1 activity in cryopreserved human hepatocytes was quantified by adding chlorzoxazone to the hepatocytes and measuring the formation of its metabolite, 6-hydroxychlorzoxazone. Colchicine at the concentrations tested did not induce CYP2E1 activity in human hepatocytes isolated from all three donors, but it did suppress CYP2E1 activity at a statistically significant level. This conclusion was based on CYP2E1 activity (67.4, 69.7, and 68.7% of the VC in Donor 1; 62.1, 72.9, and 70.2% of the VC in Donor 2; and 45.1, 44.4, and 47.7% of the VC in Donor 3) in hepatocytes treated with 0.25, 2.5, and 25 μM colchicine (Table 19). The assay method detected no chromatographic interference from colchicine or its metabolite

CYP3A4 activity in cryopreserved human hepatocytes was quantified by adding testosterone to the hepatocytes and measuring the formation of its metabolite, 61 hydroxytestosterone. The assay method detected chromatographic interference from colchicine or its metabolite since colchicine or its metabolite eluted at a retention time close to that for 61 hydroxytestosterone (data not shown). In spite of this interference, the analytical method was still able to quantitate the amount of 61 hydroxytestosterone formed following treatment with colchicine at the concentrations of 0.25 or 2.5 μM, but not at the highest concentration used 25 μM. Although the amount of 61 hydroxytestosterone formed following treatment with 25 μM of colchicine could not be quantitated due to this interference, it was judged qualitatively to be less than that following treatment with 0.25 or 2.5 μM of colchicine based on the chromatograms. Therefore, no analytical method development was recommended and it was concluded that colchicine at the concentrations tested did not induce CYP3A4 activity in human hepatocytes isolated from all three donors based on the data in Table 20, but instead suppressed CYP3A4 activity.

In summary, colchicine at the tested concentrations did not induce activities of CYP1A2, CYP2A6, CYP2B6, CYP2C8, CYP2C9, CYP2C19, CYP2D6, CYP2E1, and CYP3A4 in hepatocytes from any of the three donors. To the contrary, colchicine actually decreased the enzyme activity levels of the nine CYPs studied.

### Example 3

### Comparison of CYP1A2 Induction/Suppression Potential of Colchicine and Vinblastine in Primary Human Hepatocytes

The purpose of this study was to assess the induction and/or suppression of hepatic cytochrome P450 1A2 activity and mRNA expression by colchicine and vinblastine in primary human hepatocyte cultures prepared from three independent human donors.

Primary cultures of human hepatocytes were prepared using liver tissue from 3 human donors (Hu4021, Hu485, and Hu503; Table 1). Information on the liver tissue donors is shown below in Table 21.

TABLE 21
Liver Source Information

| Donor | | | | | | Weight | | Alcohol | Drug | Viability after |
|---|---|---|---|---|---|---|---|---|---|---|
| ID | Sex | Race | Age | Obese | Height | (lbs) | Smoking | Abuse | Abuse | Isolation |
| Hu485 | Male | Caucasian | 70 | No | 5′9″ | 190 | Yes (55 yrs) | No | No | 85% |
| Hu4021 | Female | Caucasian | 64 | No | 5′4″ | 132 | No | No | No | 84% |
| Hu503 | Male | Caucasian | 64 | No | 5′7″ | 188 | No | No | No | 90% |

Hepatocytes were isolated by a collagenase perfusion method (LeCluyse, E. L., et al. (2005) Methods Mol Biol 290, 207-229). After isolation, hepatocytes were re-suspended in Dulbecco's modified Eagle medium (DMEM; Gibco BRL, Grand Island, N.Y.) containing 5% fetal bovine serum (FBS; Gibco BRL), insulin and dexamethasone (Gibco BRL) and added to plates coated with a collagen type I substratum (BD Biosciences, Bedford, Mass.). After attachment, serum-free William's E medium containing dexamethasone, insulin, transferrin, selenium (ITS⁺, BD Biosciences) was added. Cultures of hepatocytes were maintained for 36 to 48 hours prior to initiating experiments.

Hepatocyte cultures were treated for 3 consecutive days with colchicine (0.22, 2.2, and 22 □M; Sanmar Specialty Chemicals, Ltd., Chemai, India), vinblastine (1, 10, and 100 nM; Sigma Chemical Co, St. Louis, Mo.) and the positive control CYP1A inducer 3-methylcholanthrene (3-MC; 2 □M; Sigma Chemical Co.). Dosing solutions were prepared fresh daily in cell culture medium such that the final dimethyl sulfoxide (DMSO) concentration was 0.1%. At the conclusion of the treatment period, cells were incubated with substrates and harvested for total RNA isolations.

After completion of the treatment period, medium was aspirated, the cells were rinsed once with fresh medium, and William's E containing 100 μM phenacetin, a CYP450 marker substrate for CYP1A2, was added directly to the monolayers. Plates were incubated at 37° C. in a humidified chamber while mixing on an orbital shaker for 30 minutes. At the end of the incubation period, medium samples were collected and analyzed. Analysis of the in situ CYP1A2-mediated metabolism of phenacetin to APAP was performed by liquid chromatography coupled with mass spectrometry (LC-MS/MS). Mass spectrometric data were acquired, integrated, regressed, and quantified with MASSLYNX software, version 3.4 (Micromass, Manchester, UK).

Cells in 24-well format were washed with one volume (0.5 mL) of Hank's Balanced Salt Solution and lysed by addition of 350 μL of ABI Nucleic Acid Lysis Solution (Applied Biosystems, Foster City, Calif.) and frozen at −70° C. Lysates were thawed and total RNA was isolated from each sample using an ABI 6100 Prepstation. Isolated RNA was analyzed using a Nanoprop® spectrophotometer (Wilmington, Del.) to estimate the total RNA concentrations, and RNA was stored at −70° C. For reverse transcription (RT), approximately 200 ng of pooled total RNA was converted to cDNA following the manufacturer's procedure for the High Capacity cDNA Archive Kit (Applied Biosystems, Foster City, Calif.).

CYP1A2 cDNA from human hepatocyte cultures was analyzed from each RT reaction using gene-specific TAQMAN® primer/probe sets (Applied Biosystems). Reactions with the specific primer/probes for beta-actin were analyzed as an endogenous control for CYP450 expression. Amplifications were performed on an ABI 7500 Real-Time PCR System (Applied Biosystems) in relative quantification mode for 40 amplification cycles using standard conditions for TaqMan®-based assays. Threshold cycle (CT) determinations were performed by the ABI 7500 system software for both CYP450 and endogenous control gene □-actin. Relative-fold mRNA content was determined for each treatment group relative to the endogenous control gene expression and 0.1% DMSO vehicle control for each sample using the relationship:

2^{-ΔΔC}^{_{T}}=relative-fold mRNA content.

Data (activities and mRNA) were analyzed for mean and standard error. Statistical analysis of data using multiple comparisons methods was performed with XLSTAT 2006 (Addinsoft, New York, N.Y.) to determine which treatment groups were statistically different from the vehicle control group. Initially, data were examined for homoscedasticity and normality (of residuals from the ANOVA model fitting) using Levene's and Shapiro-Wilk tests, respectively. If either normality of residuals or homoscedasticity did not exist at the p≦0.01 level, then the response variable was transformed using log transformation (natural log) to achieve both homoscedasticity and normality. Once normality of residuals and homoscedasticity were achieved at the p≦0.01 level, Dunnett's t-test was used to perform multiple comparisons analysis versus vehicle control to determine statistically significant differences at p≦0.05.

Treatment of each of the 9 sample groups is summarized in Table 22 below. 3-methylcholanthrene (3-MC) at 2 μM was used as the positive induction control. Negative control cultures were treated with media alone (in the presence or absence of 0.1% DMSO).

TABLE 22
Summary of Treatment Groups

| GROUP # | TREATMENT |
|---|---|
| 1 | Negative control (Media only) |
| 2 | Negative Control: (Media + 0.1% DMSO) |
| 3 | CYP1A Positive Control: (Media + 2 μM 3-MC) |
| 4 | Colchicine (0.22 μM) |
| 5 | Colchicine (2.2 μM) |
| 6 | Colchicine (22 μM) |
| 7 | Vinblastine (1 nM) |
| 8 | Vinblastine (10 nM) |
| 9 | Vinblastine (100 nM) |

All subsequent calculations were performed at machine precision with the software program Microsoft Excel (Redmond, Wash.). Enzyme activity (V) was calculated as shown in Equation 1:

$$V = \frac{[ X ] \times Vol}{{M}_{X} \times T \times C} , \qquad Equation 1$$

where [X] is the concentration of APAP, Vol is the volume of the incubation, M_{X} is the molecular weight of APAP, T is the time of the incubation, and C is the number of cells in the incubation volume.

To quantify the inducibility of CYP1A2, the relative fold changes were calculated as the ratio of the mean of the activity of the treated samples to the vehicle (0.1% DMSO) control activity, as shown in Equation 2, where μ is the mean activity of the replicate samples for the group designated within the parentheses.

$$Fold Induction = \frac{μ ( sample )}{μ ( DMSO )} \qquad Equation 2$$

The percent of treatment induction response as compared to the positive control induction response was calculated as shown in Equation 3.

$$\% Positive Control Response = \frac{μ ( sample ) - μ ( DMSO )}{μ ( positive control ) - μ ( DMSO )} \times 100 , \qquad Equation 3$$

where μ(sample) is the mean of the sample activities, μ(DMSO) is the mean activity of 0.1% DMSO-treated vehicle control samples, and μ(positive control) is the mean activity of positive control (3-MC)-treated samples.

Similarly, the percent suppression of enzyme activity observed relative to the vehicle control samples was calculated using Equation 4:

$$Percent Suppression = \frac{μ ( DMSO ) - μ ( sample )}{μ ( DMSO )} \times 100 \qquad Equation 4$$

The presence or absence of acute cytotoxicity to hepatocytes was determined by evaluating morphological changes and lactate dehydrogenase leakage following treatment with colchicine or vinblastine.

The morphology of the cultures was assessed on representative treatments and compared to control cultures. Cell integrity was evaluated using phase contrast microscopy; morphological alterations were noted (e.g., cell shape, cytoplasmic alterations, accumulation of vacuoles suggestive of dilated organelles and lipid droplets). Images were captured using a Zeiss Axiovert inverted research microscope equipped with phase-contrast optics, a 3 CCD camera, and imaging computer with image analysis software to document appearance observed in samples as compared to vehicle controls. No marked changes in cell morphology consistent with cytotoxicity were observed with cultures of hepatocytes treated at colchicine concentrations of 0.22-22 μM. Only the highest concentration of vinblastine, 100 nM, caused any change in cell morphology; the change observed was slight.

The inability of hepatocytes to retain intracellular enzymes is an indicator of irreversible damage to the plasma membrane. To determine membrane integrity, lactate dehydrogenase (LDH) leakage from hepatocytes was measured. Lactate dehydrogenase (LDH) leakage from hepatocytes treated with colchicine and vinblastine was measured in the culture media and compared with that of controls at 72 hours. LDH leakage was determined with the CytoTox One LDH assay system (Promega Corporation, Madison, Wis.) according to the manufacturer's instructions. The activity in the medium was expressed as percentage of the LDH activity from control cells that were completely lysed by sonication. LDH activity in the three cell lines at 22 μM colchicine was 0.8% (HU503), 6.7% (HU485), and 7.7% (HU4021), while LDH activity in the three cell lines at 100 nM vinblastine was 0.4% (HU503), 6.8% (HU485), and 3.6% (HU4021) and in the vehicle control was 0.6% (HU503), 6.0% (HU485), and 3.7% (HU4021). LDH activity observed in each of the three different cell lines varied little with colchicine or vinblastine concentration.

Enzymatic activity determinations for each of the nine sample groups are shown below in Tables 23-25, for each of the three hepatocyte donors, respectively. Levels of CYP1A2 mRNA content determined by Quantitative RT-PCR analysis is presented for each of the nine sample groups for each of the three hepatocyte donors in Tables 26-28.

TABLE 23
Phenacetin O-Dealkylation Assay for the Determination of CYP1A2 Activity Induction
in Human Hepatocytes; Hepatocyte Lot # Hu485

| | | | Activity | Mean Activity | | % | | |
|---|---|---|---|---|---|---|---|---|
| | | Conc. | (pmol/min/ | (pmol/min/ | Standard | of | Fold | % Positive |
| Treatment | Sample ID | (ng/mL) | million cells) | million cells) | Error | VC | Change | Control |
| | Hu485-0-1 | 29.7 | 8.74 | | | | | |
| | Hu485-0-2 | 29.3 | 8.62 | | | | | |
| Control (No DMSO) | Hu485-0-3 | 24.2 | 7.10 | 8.15 | 0.53 | | | |
| | Hu485-1-1 | 19.6 | 5.76 | | | | | |
| | Hu485-1-2 | 15.8 | 4.63 | | | | | |
| Control (0.1% DMSO) | Hu485-1-3 | 17.7 | 5.19 | 5.19 | 0.33 | | 1.0 | |
| | Hu485-2-1 | 2070 | 609 | | | | | |
| | Hu485-2-2 | 1550 | 455 | | | | | |
| 3-MC (2 μM) | Hu485-2-3 | 1390 | 408 | 491 | 60 | | 94.5 | 100 |
| | Hu485-3-1 | 8.71 | 2.56 | | | | | |
| | Hu485-3-2 | 8.16 | 2.40 | | | | | |
| Colchicine (0.22 μM) | Hu485-3-3 | 6.11 | 1.80 | 2.25 | 0.23 | 43.4 | 0.4 | −0.61 |
| | Hu485-4-1 | 6.59 | 1.94 | | | | | |
| | Hu485-4-2 | 8.45 | 2.48 | | | | | |
| Colchicine (2.2 μM) | Hu485-4-3 | 8.31 | 2.44 | 2.29 | 0.18 | 44.1 | 0.4 | −0.60 |
| | Hu485-5-1 | 10.1 | 2.96 | | | | | |
| | Hu485-5-2 | 6.27 | 1.84 | | | | | |
| Colchicine (22 μM) | Hu485-5-3 | 5.95 | 1.75 | 2.18 | 0.39 | 42.0 | 0.4 | −0.62 |
| | Hu485-6-1 | 41.7 | 12.3 | | | | | |
| | Hu485-6-2 | 46.9 | 13.8 | | | | | |
| Vinblastine (1 nM) | Hu485-6-3 | 45.1 | 13.3 | 13.1 | 0.4 | 252 | 2.5 | 1.6 |
| | Hu485-7-1 | 43.1 | 12.7 | | | | | |
| | Hu485-7-2 | 56.2 | 16.5 | | | | | |
| Vinblastine (10 nM) | Hu485-7-3 | 42.6 | 12.5 | 13.9 | 1.3 | 268 | 2.7 | 1.8 |
| | Hu485-8-1 | 33.8 | 9.93 | | | | | |
| | Hu485-8-2 | 32.7 | 9.61 | | | | | |
| Vinblastine (100 nM) | Hu485-8-3 | 27.7 | 8.15 | 9.23 | 0.55 | 178 | 1.8 | 0.83 |

TABLE 24
Phenacetin O-Dealkylation Assay for the Determination of CYP1A2 Activity
Induction in Human Hepatocytes; Hepatocyte Lot # Hu4021

| | | | | Activity | Mean Activity | | % | | |
|---|---|---|---|---|---|---|---|---|---|
| | | | Conc. | (pmol/min/ | (pmol/min/ | Std | of | Fold | % Positive |
| Treatment | Sample ID | | (ng/mL) | million cells) | million cells) | Error | VC | Change | Control |
| Control (No | Hu4021-0-1 | | 10.9 | 3.20 | | | | | |
| DMSO) | Hu4021-0-2 | | 18.9 | 5.55 | | | | | |
| | Hu4021-0-3 | | 16.0 | 4.69 | | | | | |
| | Hu4021-0-4 | | 14.2 | 4.17 | | | | | |
| | Hu4021-0-5 | | 12.0 | 3.52 | | | | | |
| | Hu4021-0-6 | | 15.1 | 4.44 | 4.26 | 0.35 | | | |
| Control (0.1% | Hu4021-1-1 | | 21.3 | 6.26 | | | | | |
| DMSO) | Hu4021-1-2 | | 26.3 | 7.72 | | | | | |
| | Hu4021-1-3 | | 21.2 | 6.25 | | | | | |
| | Hu4021-1-4 | | 18.1 | 5.32 | | | | | |
| | Hu4021-1-5 | | 23.6 | 6.95 | | | | | |
| | Hu4021-1-6 | | 31.0 | 9.11 | 6.93 | 0.54 | | 1.0 | |
| 3-MC (2 μM) | Hu4021-2-1 | | 1160 | 341 | | | | | |
| | Hu4021-2-2 | | 1060 | 311 | | | | | |
| | Hu4021-2-3 | | 1170 | 343 | | | | | |
| | Hu4021-2-4 | | 1150 | 339 | | | | | |
| | Hu4021-2-5 | | 1280 | 376 | | | | | |
| | Hu4021-2-6* | N26 | 1740 | 511 | 342 | 10 | | 49.3 | 100 |
| Colchicine | Hu4021-3-1 | | 12.2 | 3.60 | | | | | |
| (0.22 μM) | Hu4021-3-2 | | 12.4 | 3.63 | | | | | |
| | Hu4021-3-3 | | 8.25 | 2.43 | 3.22 | 0.40 | 46.5 | 0.5 | −1.1 |
| Colchicine | Hu4021-4-1 | | 6.39 | 1.88 | | | | | |
| (2.2 μM) | Hu4021-4-2 | | 10.5 | 3.08 | | | | | |
| | Hu4021-4-3 | | 6.37 | 1.87 | 2.28 | 0.40 | 32.9 | 0.3 | −1.4 |
| Colchicine | Hu4021-5-1 | | 8.71 | 2.56 | | | | | |
| (22 μM) | Hu4021-5-2 | | 7.21 | 2.12 | | | | | |
| | Hu4021-5-3 | | 6.34 | 1.86 | 2.18 | 0.20 | 31.5 | 0.3 | −1.4 |
| Vinblastine | Hu4021-6-1 | | 27.2 | 7.99 | | | | | |
| (1 nM) | Hu4021-6-2 | | 22.0 | 6.47 | | | | | |
| | Hu4021-6-3 | | 24.0 | 7.06 | 7.17 | 0.44 | 103.5 | 1.0 | 0.1 |
| Vinblastine | Hu4021-7-1 | | 65.4 | 19.2 | | | | | |
| (10 nM) | Hu4021-7-2 | | 61.4 | 18.0 | | | | | |
| | Hu4021-7-3 | | 55.1 | 16.2 | 17.8 | 0.9 | 256.9 | 2.6 | 3.2 |
| Vinblastine | Hu4021-8-1 | | 17.7 | 5.20 | | | | | |
| (100 nM) | Hu4021-8-2 | | 18.3 | 5.39 | | | | | |
| | Hu4021-8-3 | | 19.1 | 5.63 | 5.41 | 0.12 | 78.1 | 0.8 | −0.5 |

N26 - NOT INCLUDED IN CALCULATIONS; OUTLIER BASED ON GRUBB'S TEST.

TABLE 25
Phenacetin O-Dealkylation Assay for the Determination of CYP1A2 Activity Induction
in Human Hepatocytes; Hepatocyte Lot # Hu503

| | | | | Activity | Mean Activity | | % | | |
|---|---|---|---|---|---|---|---|---|---|
| | | | Conc. | (pmol/min/ | (pmol/min/ | Standard | of | Fold | % Positive |
| Treatment | Sample ID | | (ng/mL) | million cells) | million cells) | Error | VC | Change | Control |
| | Hu503-0-1 | | 57.3 | 16.8 | | | | | |
| | Hu503-0-2 | | 59.7 | 17.6 | | | | | |
| | Hu503-0-3 | | 58.8 | 17.3 | | | | | |
| | Hu503-0-4 | | 69.2 | 20.3 | | | | | |
| | Hu503-0-5 | | 58.8 | 17.3 | | | | | |
| Control (No DMSO) | Hu503-0-6 | | 52.7 | 15.5 | 17.5 | 0.6 | | | |
| | Hu503-1-1 | | 55.5 | 16.3 | | | | | |
| | Hu503-1-2 | | 53.5 | 15.7 | | | | | |
| | Hu503-1-3 | | 62.7 | 18.4 | | | | | |
| | Hu503-1-4 | | 53.9 | 15.9 | | | | | |
| | Hu503-1-5 | | 57.0 | 16.7 | | | | | |
| Control (0.1% DMSO) | Hu503-1-6 | | 58.6 | 17.2 | 16.7 | 0.4 | | 1.0 | |
| | Hu503-2-1 | E2 | 2270 | 666 | | | | | |
| | Hu503-2-2 | E2 | 2000 | 589 | | | | | |
| | Hu503-2-3 | E2 | 2070 | 609 | | | | | |
| | Hu503-2-4 | | 1950 | 573 | | | | | |
| | Hu503-2-5 | E2 | 2070 | 608 | | | | | |
| 3-MC (2 μM) | Hu503-2-6 | E2 | 2270 | 667 | 619 | 16 | | 37.0 | 100 |
| | Hu503-3-1 | | 44.9 | 13.2 | | | | | |
| | Hu503-3-2 | | 39.1 | 11.5 | | | | | |
| Colchicine (0.22 μM) | Hu503-3-3 | | 40.0 | 11.8 | 12.2 | 0.5 | 73.1 | 0.7 | −0.76 |
| | Hu503-4-1 | | 28.0 | 8.22 | | | | | |
| | Hu503-4-2 | | 34.4 | 10.1 | | | | | |
| Colchicine (2.2 μM) | Hu503-4-3 | | 34.4 | 10.1 | 9.49 | 0.63 | 56.8 | 0.6 | −1.2 |
| | Hu503-5-1 | | 31.3 | 9.21 | | | | | |
| | Hu503-5-2 | | 29.3 | 8.63 | | | | | |
| Colchicine (22 μM) | Hu503-5-3 | | 21.6 | 6.34 | 8.06 | 0.88 | 38.0 | 0.5 | −1.4 |
| | Hu503-6-1 | | 72.9 | 21.4 | | | | | |
| | Hu503-6-2 | | 68.0 | 20.0 | | | | | |
| Vinblastine (1 nM) | Hu503-6-3 | | 63.6 | 18.7 | 20.0 | 0.8 | | 1.2 | 0.55 |
| | Hu503-7-1 | | 68.6 | 20.2 | | | | | |
| | Hu503-7-2 | | 69.0 | 20.3 | | | | | |
| Vinblastine (10 nM) | Hu503-7-3 | | 65.3 | 19.2 | 19.9 | 0.3 | | 1.2 | 0.53 |
| | Hu503-8-1 | | 58.8 | 17.3 | | | | | |
| | Hu503-8-2 | | 55.0 | 16.2 | | | | | |
| Vinblastine (100 nM) | Hu503-8-3 | | 57.8 | 17.0 | 16.8 | 0.3 | | 1.0 | 0.02 |

E2 - ESTIMATED VALUE; ABOVE THE ULOQ.

TABLE 26
Quantitative RT-PCR Analysis of CYP1A2 mRNA Content Induction In Human
Hepatocytes; Hepatocyte Lot# Hu485

| | Sample | Relative Fold | Mean Relative Fold | Standard | Percent of Adjusted |
|---|---|---|---|---|---|
| Treatment | ID | mRNA Content | mRNA Content | Error | Positive Control |
| DMSO (0.1%) | Hu485-1 | 1.00 | 0.817 | 0.14 | 0.00 |
| | Hu485-1 | 0.537 | | | |
| | Hu485-1 | 0.913 | | | |
| 3-MC (2 μM) | Hu485-2 | 361 | 396 | 25.50 | 100.00 |
| | Hu485-2 | 445 | | | |
| | Hu485-2 | 380 | | | |
| Colchicine (0.22 μM) | Hu485-3 | 0.056 | 0.057 | 0.003 | −0.19 |
| | Hu485-3 | 0.054 | | | |
| | Hu485-3 | 0.063 | | | |
| Colchicine (2.2 μM) | Hu485-4 | 0.064 | 0.056 | 0.004 | −0.19 |
| | Hu485-4 | 0.055 | | | |
| | Hu485-4 | 0.050 | | | |
| Colchicine (22 μM) | Hu485-5 | 0.036 | 0.029 | 0.004 | −0.20 |
| | Hu485-5 | 0.024 | | | |
| | Hu485-5 | 0.029 | | | |
| Vinblastine (1 nM) | Hu485-6 | 6.18 | 5.68 | 0.34 | 1.23 |
| | Hu485-6 | 5.02 | | | |
| | Hu485-6 | 5.83 | | | |
| Vinblastine (10 nM) | Hu485-7 | 5.88 | 5.47 | 0.40 | 1.18 |
| | Hu485-7 | 4.66 | | | |
| | Hu485-7 | 5.86 | | | |
| Vinblastine (100 nM) | Hu485-8 | 3.48 | 3.59 | 0.09 | 0.70 |
| | Hu485-8 | 3.52 | | | |
| | Hu485-8 | 3.78 | | | |

TABLE 27
Quantitative RT-PCR Analysis of CYP1A2 mRNA Content Induction In Human
Hepatocytes; Hepatocyte Lot# Hu4021

| | | Relative Fold mRNA | Mean Relative Fold | Standard | Percent of Positive |
|---|---|---|---|---|---|
| Treatment | Sample ID | Content | mRNA Content | Error | Control |
| DMSO (0.1%) | Hu4021-1 | 1.00 | 0.894 | 0.07 | 0.00 |
| | Hu4021-1 | 0.753 | | | |
| | Hu4021-1 | 0.930 | | | |
| 3-MC (2 μM) | Hu4021-2 | 68.7 | 71.9 | 2.97 | 100.00 |
| | Hu4021-2 | 69.1 | | | |
| | Hu4021-2 | 77.8 | | | |
| Colchicine (0.22 μM) | Hu4021-3 | 0.071 | 0.047 | 0.01 | −1.19 |
| | Hu4021-3 | 0.023 | | | |
| | Hu4021-3 | 0.047 | | | |
| Colchicine (2.2 μM) | Hu4021-4 | 0.017 | 0.015 | 0.002 | −1.24 |
| | Hu4021-4 | 0.011 | | | |
| | Hu4021-4 | 0.016 | | | |
| Colchicine (22 μM) | Hu4021-5 | 0.025 | 0.026 | 0.004 | −1.22 |
| | Hu4021-5 | 0.020 | | | |
| | Hu4021-5 | 0.033 | | | |
| Vinblastine (1 nM) | Hu4021-6 | 0.259 | 0.229 | 0.03 | −0.94 |
| | Hu4021-6 | 0.162 | | | |
| | Hu4021-6 | 0.264 | | | |
| Vinblastine (10 nM) | Hu4021-7 | 1.70 | 1.88 | 0.19 | 1.38 |
| | Hu4021-7 | 1.68 | | | |
| | Hu4021-7 | 2.25 | | | |
| Vinblastine (100 nM) | Hu4021-8 | 0.357 | 0.320 | 0.02 | −0.81 |
| | Hu4021-8 | 0.280 | | | |
| | Hu4021-8 | 0.323 | | | |

TABLE 28
Quantitative RT-PCR Analysis of CYP1A2 mRNA Content Induction In Human
Hepatocytes; Hepatocyte Lot# Hu503

| | | Relative Fold mRNA | Mean Relative Fold | Standard | Percent of Positive |
|---|---|---|---|---|---|
| Treatment | Sample ID | Content | mRNA Content | Error | Control |
| DMSO (0.1%) | Hu503-1 | 1.00 | 0.914 | 0.04 | 0.00 |
| | Hu503-1 | 0.867 | | | |
| | Hu503-1 | 0.875 | | | |
| 3-MC (2 μM) | Hu503-2 | 167 | 161 | 12.74 | 100.00 |
| | Hu503-2 | 137 | | | |
| | Hu503-2 | 180 | | | |
| Colchicine (0.22 μM) | Hu503-3 | 0.030 | 0.025 | 0.01 | −0.55 |
| | Hu503-3 | 0.015 | | | |
| | Hu503-3 | 0.031 | | | |
| Colchicine (2.2 μM) | Hu503-4 | 0.015 | 0.015 | 0.001 | −0.56 |
| | Hu503-4 | 0.013 | | | |
| | Hu503-4 | 0.016 | | | |
| Colchicine (22 μM) | Hu503-5 | 0.016 | 0.018 | 0.001 | −0.56 |
| | Hu503-5 | 0.018 | | | |
| | Hu503-5 | 0.020 | | | |
| Vinblastine (1 nM) | Hu503-6 | 4.18 | 3.604 | 0.29 | 1.68 |
| | Hu503-6 | 3.22 | | | |
| | Hu503-6 | 3.40 | | | |
| Vinblastine (10 nM) | Hu503-7 | 3.70 | 3.31 | 0.23 | 1.49 |
| | Hu503-7 | 3.32 | | | |
| | Hu503-7 | 2.90 | | | |
| Vinblastine (100 nM) | Hu503-8 | 1.01 | 0.896 | 0.06 | −0.01 |
| | Hu503-8 | 0.816 | | | |
| | Hu503-8 | 0.865 | | | |

A marked induction of CYP1A2-catalyzed APAP formation from phenacetin was observed with the positive control 3-MC (varying from 37.1 to 94.6-fold induction relative to the activity of the vehicle control (medium+0.1% DMSO) for the three hepatocyte preparations), demonstrating that the hepatocyte cultures were responding appropriately to a prototypical CYP1A inducer (Tables 25-27). At the concentrations tested, no induction of CYP1A activity was observed in human hepatocytes treated with colchicine at any of the concentrations examined, consistent with the data in Examples 1 and 2. Instead, a marked suppression of CYP1A2 enzyme activity was observed in all three preparations of human hepatocyte cultures, as observed in the Example 2 experiment shown in Table 13. In the current experiments, suppression ranged from 27.3% to 68.5% lower than the vehicle control for CYP1A2 enzyme activity (Tables 25-27). Observed CYP1A2 activity as a percent of the VC activity after treatment with 0.22, 2.2, and 22 μM colchicine, respectively was 43.4%, 44.1%, and 42.0% for Donor Hu485; 46.5%, 32.9%, and 31.5% for Donor Hu4021; and 73.1%, 56.8%, and 38.0% for Donor Hu503. These substantial reductions from the VC in hepatocyte cultures from three different human donors confirm the suppression of CYP1A2 enzyme activity observed in Donors 1-3 (Table 13) in Example 2. In contrast, for vinblastine, the alternate tubulin network disrupting agent tested, a very small induction (relative to the positive control response) was observed that ranged from −0.46 to 3.2% of the fold-change of the positive control.

A marked induction of CYP1A2 mRNA content was observed with the positive control inducer, 3-MC, over the vehicle control. The mean relative fold mRNA content ranged from 71.9 to 396 for the three hepatocyte preparations (Tables 28-30), demonstrating that the hepatocyte cultures were responding appropriately to the prototypical CYP1A inducer. No induction of CYP1A2 mRNA content was observed with colchicine treatment at any concentration (Tables 28-30), consistent with the observed lack of induction of enzyme activity by colchicine. Instead, a marked suppression of mRNA content was observed in all three preparations of human hepatocyte cultures. The mean relative fold mRNA content observed after treatment with colchicine at 0.22, 2.2, and 22 μM was only 0.057, 0.056, and 0.029 for Donor Hu485 (Table 28); 0.047, 0.015, and 0.026 for Donor Hu4021 (Table 29); and 0.025, 0.015, and 0.018 for Donor Hu503 (Table 30).

In contrast, for vinblastine, no marked suppression of enzyme activity or mRNA expression was observed with vinblastine at the concentrations examined. Instead, either a slight suppression or a small induction (with both suppression and induction expressed relative to the positive control induction) was observed in all three donor preparations of primary human hepatocyte cultures. The observed effect ranged from −0.94% to 1.68% of the positive control response.

Therefore, colchicine has a concentration-dependent suppressive effect on CYP1A2 mRNA expression and CYP1A2 enzyme activity at concentrations from 0.22 to 22 μM, while vinblastine, another tubulin disrupting compound, does not suppress CYP1A2 expression. Cytotoxicity did not appear to be the cause of the suppression observed at the range of colchicine concentrations studied.

### Example 4

### Metabolic Phenotyping of Colchicine

The purpose of this study was to identify the cytochrome P450 enzymes involved in the in vitro metabolism of Colchicine.

Human liver microsomes pooled from 15 individuals (male and female) (Pool HMMC-PL020; CellzDirect, Inc., In Vitro Products and Services Division) were utilized in some experiments of this study. This pool of human liver microsomes was characterized by CellzDirect, Inc. with respect to donor medical history, major cytochrome P450 enzyme activities and kinetic parameters, as well as for polymorphic forms for CYP2C9, CYP2C19, and CYP2D6 present in the individual donors of the pool.

Colchicine (MW=399.43, Lot # COL0206002 purity=97.05%) stock solutions at 430 μM were prepared in methanol and stored at −20° C. Stocks were diluted daily in the appropriate buffers such that the final organic solvent concentration was <1%.

Potassium phosphate monobasic, potassium phosphate dibasic, NADPH tetrasodium salts, and other reagents were purchased from Sigma Chemical Co. or equivalent vendors. Methanol (HPLC grade), water (HPLC grade), ethyl acetate, and other solvents were purchased from Fisher, Burdick & Jackson, J. T. Baker, Mallinckrodt, or equivalent vendors. All inhibitors were of the highest purity available. Individual suppliers are as follows: furafylline, pilocarpine, thio-TEPA, quercetin, sulfaphenazole, ticlopidine, quinidine, 4 methylpyrazole, and ketoconazole were obtained from Sigma Chemical Co.

A validated isocratic LC-MS/MS was developed to allow for chromatographic resolution and quantitation of colchicine contained within an incubation matrix. The following LC-MS/MS method was used for Colchicine quantitation:

TABLE 29
LC/MS method for colchicine quantitation.

| | |
|---|---|
| Substrate: Metabolite: | Colchicine |
| Standard Metabolite Range: | 4.31 to 552 μM |
| Mobile Phase (isocratic): | 50% Methanol, 50% 1 mM Ammonium |
| | Acetate Buffer, 0.1% Trifluroacetic Acid |
| | (TFA) |
| Detection Method: | LC-MS/MS |
| HPLC Column: | AQ12 |
| Flow rate (approx.): | 0.3 mL/min |
| Source: | Electrospray (positive ion) |
| Run Time (approx.): | 2 minutes |
| MRM (Colchicine): | 400 → 310 |
| Quantitation: | Least Squares Regression 1/X Weighting |

For this example, Micromass MASSLYNX® software (version 3.4, Manchester, UK) was used to collect and process chromatographic data. Data were graphed with the software program Microsoft EXCE® 2003 (Redmond, Wash.). Percent turnover of colchicine was calculated using Microsoft EXCEL at machine precision, using the following equation:

% Turnover=100−{[TA(final)]/[mean of TA(0 min)]}×100,

where TA(sample group)=Test Article (i.e., colchicine) and the particular sample group is noted within the parentheses.

Incubations of colchicine with pooled human liver microsomes were performed to establish appropriate protein concentrations and time points for Colchicine turnover. In particular, colchicine at 43 and 430 nM was incubated with 0, 0.02, 0.05, 0.1, 0.25 and 0.5 mg/mL pooled human liver microsomal protein for 30 minutes at 37° C. In addition, 43 and 430 nM of colchicine was incubated with 0.1 mg/mL pooled human liver microsomal protein for 0, 5, 10, 20, 40, and 60 minutes at 37° C. Incubation mixtures were prepared in 0.1 M phosphate buffer, pH 7.4. The reactions were initiated by addition of 1 mM NADPH. Reactions were performed in triplicate and were terminated at the appropriate time points by addition of 1 volume (relative to total reaction volume) of methanol. Negative controls (either no NADPH or heat-treated microsomes) were included to account for any non-enzymatic dependent reactions. The samples were centrifuged at approximately 3000 rpm and the clear supernatant was transferred to a clean tube and analyzed by the LC-MS/MS method described above.

Incubations with 43 and 430 nM colchicine with 0.1 mg/mL of pooled human liver microsomal protein for 0, 5, 10, 20, 40 and 60 minutes resulted in a decrease in colchicine concentration of less than 5% after 60 minutes incubations. These changes in colchicine concentration were not consistently greater in magnitude than the sample-to-sample variability.

Based on this matrix of data, a pooled human liver microsomal protein concentration of 1 mg/mL, and an incubation time of 60 minutes were used in subsequent experiments using the microsomal system.

Experiments were then performed to identify CYP450 isoform(s) involved in the metabolism of colchicine in a microsomal system using selective CYP chemical inhibitors. The microsomal system approximates the in vivo distribution of hepatic enzymes.

Isoform-selective chemical inhibitors were used to evaluate the effects of individual CYP450s in human liver microsomes on the metabolism of colchicine. The isoform-selective chemical inhibitors and concentrations used are detailed in Table 30 below.

TABLE 30
Isoform-selective chemical inhibitors and concentrations

| P450 Isoform | Selective chemical inhibitor | Concentration | |
|---|---|---|---|
| CYP1A2 | Furafylline | 50 | μM |
| CYP2A6 | Pilocarpine | 100 | μM |
| CYP2B6 | Thio-TEPA | 75 | μM |
| CYP2C8 | Quercetin | 10 | μM |
| CYP2C9 | Sulfaphenazole | 20 | μM |
| CYP2C19 | Ticlopidine | 1 | μM |
| CYP2D6 | Quinidine | 10 | μM |
| CYP2E1 | 4-Methylpyrazole | 500 | μM |
| CYP3A4 | Ketoconazole | 1 | μM |

Results are provided in Tables 31 and 32. Colchicine was only weakly metabolized by NADPH-dependent CYP450s with approximately less than 5% disappearance at the conditions examined. As turnover of colchicine in microsomes was found to be below the experimental noise, determinations of percent inhibition of turnover were not determined in these experiments.

TABLE 31
Chemical Inhibitor Data in Human Liver Microsomes with 43 nM Colchicine

| | | | Conc. | Mean | | % Turnover | |
|---|---|---|---|---|---|---|---|
| Inhibitor | CYP | Sample ID | (nM) | Conc. (nM) | % Turnover | Mean | Std. Error |
| | | 50 nM-T0-P1.0-1 | 43.8 | | | | |
| | | 50 nM-T0-P1.0-2 | 47.4 | | | | |
| | 0 min. | 50 nM-T0-P1.0-3 | 45.8 | 45.7 | | | |
| | | 50 nM-T60-P1.0-1 | 37.1 | | 18.7 | | |
| | | 50 nM-T60-P1.0-2 | 45.5 | | 0.30 | | |
| | 60 min. | 50 nM-T60-P1.0-3 | 48.7 | 43.8 | −6.5 | 4.2 | 7.6 |
| | | 50 nM-T60-P1.0-F-1 | 42.8 | | 6.2 | | |
| | | 50 nM-T60-P1.0-F-2 | 48.1 | | −5.2 | | |
| Furafylline | 1A2 | 50 nM-T60-P1.0-F-3 | 45.7 | 45.5 | 0.06 | 0.35 | 3.29 |
| | | 50 nM-T60-P1.0-P-1 | 45.8 | | −0.26 | | |
| | | 50 nM-T60-P1.0-P-2 | 48.8 | | −6.8 | | |
| Pilocarpine | 2A6 | 50 nM-T60-P1.0-P-3 | 45.8 | 46.8 | −0.29 | −2.4 | 2.2 |
| | | 50 nM-T60-P1.0-TT-1 | 46.3 | | −1.4 | | |
| | | 50 nM-T60-P1.0-TT-2 | 47.8 | | −4.6 | | |
| Thio-TEPA | 2B6 | 50 nM-T60-P1.0-TT-3 | 50.1 | 48.1 | −9.6 | −5.2 | 2.4 |
| | | 50 nM-T60-P1.0-Qr-1 | 45.8 | | −0.31 | | |
| | | 50 nM-T60-P1.0-Qr-2 | 46.6 | | −2.0 | | |
| Quercetin | 2C8 | 50 nM-T60-P1.0-Qr-3 | 45.3 | 45.9 | 0.79 | −0.52 | 0.83 |
| | | 50 nM-T60-P1.0-S-1 | 48.5 | | −6.1 | | |
| | | 50 nM-T60-P1.0-S-2 | 48.3 | | −5.8 | | |
| Sulfaphenazole | 2C9 | 50 nM-T60-P1.0-S-3 | 45.0 | 47.3 | 1.5 | −3.5 | 2.5 |
| | | 50 nM-T60-P1.0-Ti-1 | 52.4 | | −14.8 | | |
| | | 50 nM-T60-P1.0-Ti-2 | 48.6 | | −6.4 | | |
| Ticlopidine | 2C19 | 50 nM-T60-P1.0-Ti-3 | 48.4 | 49.8 | −5.9 | −9.0 | 2.9 |
| | | 50 nM-T60-P1.0-Qi-1 | 50.0 | | −9.5 | | |
| | | 50 nM-T60-P1.0-Qi-2 | 47.7 | | −4.4 | | |
| Quinidine | 2D6 | 50 nM-T60-P1.0-Qi-3 | 48.6 | 48.8 | −6.5 | −6.8 | 1.5 |
| | | 50 nM-T60-P1.0-4MP-1 | 45.6 | | 0.24 | | |
| | | 50 nM-T60-P1.0-4MP-2 | 43.4 | | 4.9 | | |
| 4-Methylpyrazole | 2E1 | 50 nM-T60-P1.0-4MP-3 | 43.7 | 44.2 | 4.3 | 3.2 | 1.5 |
| | | 50 nM-T60-P1.0-K-1 | 48.4 | | −5.9 | | |
| | | 50 nM-T60-P1.0-K-2 | 51.8 | | −13.3 | | |
| Ketoconazole | 3A4 | 50 nM-T60-P1.0-K-3 | 48.4 | 49.5 | −6.0 | −8.4 | 2.5 |

TABLE 32
Chemical Inhibitor Data in Human Liver Microsomes with 430 nM
Colchicine

| | | | | Conc. | Mean | % | % Turnover | |
|---|---|---|---|---|---|---|---|---|
| Inhibitor | CYP | Sample ID | | (nM) | Conc. (nM) | Turnover | Mean | Std. Error |
| | | 500 nM-T0-P1.0-1 | | 468 | | | | |
| | | 500 nM-T0-P1.0-2 | | 463 | | | | |
| | 0 min. | 500 nM-T0-P1.0-3 | | 464 | 465 | | | |
| | | 500 nM-T60-P1.0-1 | | 458 | | 1.5 | | |
| | | 500 nM-T60-P1.0-2 | | 456 | | 1.9 | | |
| | 60 min. | 500 nM-T60-P1.0-3 | | 441 | 452 | 5.2 | 2.8 | 1.2 |
| | | 500 nM-T60-P1.0-F-1 | | 441 | | 5.2 | | |
| | | 500 nM-T60-P1.0-F-2 | | 457 | | 1.6 | | |
| Furafylline | 1A2 | 500 nM-T60-P1.0-F-3 | | 463 | 454 | 0.45 | 2.4 | 1.4 |
| | | 500 nM-T60-P1.0-P-1 | | 468 | | −0.77 | | |
| | | 500 nM-T60-P1.0-P-2 | | 442 | | 4.9 | | |
| Pilocarpine | 2A6 | 500 nM-T60-P1.0-P-3 | | 456 | 456 | 1.8 | 2.0 | 1.6 |
| | | 500 nM-T60-P1.0-TT-1 | | 461 | | −0.77 | | |
| | | 500 nM-T60-P1.0-TT-2 | | 466 | | −0.33 | | |
| Thio-TEPA | 2B6 | 500 nM-T60-P1.0-TT-3 | | 462 | 463 | 0.54 | 0.33 | 0.34 |
| | | 500 nM-T60-P1.0-Qr-1 | | 473 | | −1.9 | | |
| | | 500 nM-T60-P1.0-Qr-2 | | 470 | | −1.1 | | |
| Quercetin | 2C8 | 500 nM-T60-P1.0-Qr-3 | | 470 | 471 | −1.1 | −1.4 | 0.3 |
| | | 500 nM-T60-P1.0-S-1 | | 448 | | 3.5 | | |
| | | 500 nM-T60-P1.0-S-2 | | 541 | | −16.5 | | |
| Sulfaphenazole | 2C9 | 500 nM-T60-P1.0-S-3 | | 522 | 504 | −12.3 | −8.4 | 6.1 |
| | | 500 nM-T60-P1.0-Ti-1 | | 493 | | −6.1 | | |
| | | 500 nM-T60-P1.0-Ti-2 | | 502 | | −8.1 | | |
| Ticlopidine | 2C19 | 500 nM-T60-P1.0-Ti-3 | N14 | NA | 498 | NA | −7.1 | 1.0 |
| | | 500 nM-T60-P1.0-Qi-1 | | 479 | | −3.0 | | |
| | | 500 nM-T60-P1.0-Qi-2 | | 469 | | −0.85 | | |
| Quinidine | 2D6 | 500 nM-T60-P1.0-Qi-3 | | 474 | 474 | −2.1 | −2.0 | 0.6 |
| | | 500 nM-T60-P1.0-4MP-1 | | 462 | | 0.61 | | |
| | | 500 nM-T60-P1.0-4MP-2 | | 476 | | −2.3 | | |
| 4-Methylpyrazole | 2E1 | 500 nM-T60-P1.0-4MP-3 | | 466 | 468 | −0.30 | −0.68 | 0.88 |
| | | 500 nM-T60-P1.0-K-1 | | 489 | | −5.3 | | |
| | | 500 nM-T60-P1.0-K-2 | | 485 | | −4.4 | | |
| Ketoconazole | 3A4 | 500 nM-T60-P1.0-K-3 | | 487 | 487 | −4.7 | −4.8 | 0.3 |

N14 - NOT INCLUDED IN CALCULATIONS; NO PEAK DETECTED.

Experiments with individual human recombinant cytochrome P450 enzymes were conducted to determine which specific CYP450s metabolize colchicine.

Commercially available microsomes from baculovirus-infected insect cells containing singly-expressed recombinant human CYP enzymes and cDNA-expressed human cytochrome p450 oxidoreductase [BD SUPERSOMES® Enzymes; BD Biosciences Discovery Labware (Woburn, Mass.)] were used. For CYP2A6, CYP2C9, CYP2C19, and CYP2E1, the SUPERSOMES® also expressed human cytochrome b5 in addition to human cytochrome p450 oxidoreductase and the human CYP isozyme. For CYP2C9 and CYP2D6, SUPERSOMES singly-expressing different allelic variants of the cytochrome p450 isozyme were commercially available. For each of CYP2C9 and CYP2D6, only the *1 allele was tested in these experiments.

Incubation mixtures containing CYP1A2, CYP2A6, CYP2B6, CYP2C8, CYP2C9, CYP2C19, CYP2D6, CYP2E1, or CYP3A4 expressed enzymes at 5 or 20 pmol cytochrome P450 were incubated in 0.1 M potassium phosphate buffer, pH 7.4 with β-NADPH (1 mM) and with colchicine for 60 minutes at 37° C. The incubations, performed in triplicate, were terminated by addition of approximately 1 volume (relative to the total reaction volume) of methanol. The samples were extracted and analyzed by LC-MS/MS, as described above. The rates of colchicine depletion by the CYP were compared depletion by a control for native activity (microsomes expressing no recombinant CYP450 enzyme).

An additional control for each isoform included verification that the isoform was active by incubating mixtures with the universal CYP substrate, phenanthrene, and monitoring turnover of phenanthrene fluorometrically at 254 nm (excitation) and 378 nm (emission). The universal CYP450 positive control substrate phenanthrene, examined with each recombinant enzyme to ensure proper activity, confirmed that each recombinant enzyme microsomal preparation was active (data not shown).

The results measuring colchicine disappearance at either 43 or 430 nM in incubations containing either 5 or 20 pmol of a recombinant cytochrome P450 isozyme is summarized below in Tables 33-34.

TABLE 33
Metabolism of Colchicine (43 nM) by Expressed Recombinant Human Cytochromes
P450 at 5 or 20 pmol

| | 5 pmol cyp | | | % Turnover | Std | 20 pmol cyp | | | % Turnover | |
|---|---|---|---|---|---|---|---|---|---|---|
| 43 nM col | [col], nM | Mean | Std dev | Mean | Error | [col], nM | Mean | Std dev | Mean | Std Error |
| control | 54.6 | | | | | 54.6 | | | | |
| control | 55.3 | 55.5 | 1.01 | | | 55.3 | 55.5 | 1.01 | | |
| control | 56.6 | | | | | 56.6 | | | | |
| 1A2 | 56.9 | 55.1 | 1.70 | | | 57.1 | 55.2 | 1.72 | | |
| 1A2 | 53.5 | | | | | 53.7 | | | | |
| 1A2 | 55.0 | | | 0.68 | 1.77 | 54.9 | | | 0.46 | 1.81 |
| 2A6 | 55.1 | 54.5 | 3.39 | | | 54.9 | 53.6 | 1.68 | | |
| 2A6 | 57.5 | | | | | 54.2 | | | | |
| 2A6 | 50.8 | | | 1.8 | 3.5 | 51.7 | | | 3.4 | 1.7 |
| 2B6 | 54.0 | 54.6 | 1.16 | | | 48.9 | 48.1 | 0.917 | | |
| 2B6 | 55.9 | | | | | 47.1 | | | | |
| 2B6 | 53.8 | | | 1.7 | 1.2 | 48.3 | | | 13.3 | 1.0 |
| 2C8 | 57.6 | 54.4 | 3.25 | | | 50.2 | 50.8 | 3.69 | | |
| 2C8 | 54.4 | | | | | 47.5 | | | | |
| 2C8 | 51.1 | | | 2.1 | 3.4 | 54.8 | | | 8.4 | 3.8 |
| 2C9 | 48.2 | 51.1 | 2.55 | | | 46.2 | 48.3 | 1.89 | | |
| 2C9 | 52.8 | | | | | 49.8 | | | | |
| 2C9 | 52.4 | | | 7.9 | 2.7 | 49.0 | | | 12.9 | 2.0 |
| 2C19 | 51.9 | 55.3 | 2.97 | | | 49.1 | 50.4 | 2.08 | | |
| 2C19 | 57.4 | | | | | 52.8 | | | | |
| 2C19 | 56.6 | | | 0.37 | 3.10 | 49.3 | | | 9.1 | 2.2 |
| 2D6 | 57.5 | 52.6 | 4.76 | | | 56.9 | 55.7 | 1.5o | | |
| 2D6 | 52.3 | | | | | 54.0 | | | | |
| 2D6 | 48.0 | | | 5.2 | 5.0 | 56.1 | | | −0.35 | 1.54 |
| 2E1 | 49.7 | 53.0 | 2.86 | | | 55.9 | 54.6 | 1.67 | | |
| 2E1 | 54.7 | | | | | 52.7 | | | | |
| 2E1 | 54.6 | | | 4.5 | 3.0 | 55.1 | | | 1.6 | 1.7 |
| 3A4 | 53.9 | 55.4 | 1.3 | | | 43.4 | 44.2 | 0.721 | | |
| 3A4 | 56.1 | | | | | 44.4 | | | | |
| 3A4 | 56.2 | | | 0.19 | 1.33 | 44.8 | | | 20.3 | 0.7 |

TABLE 34
Metabolism of Colchicine (430 nM) by Expressed Recombinant Human Cytochromes
P450 at 5 or 20 pmol

| 430 nM | 5 pm cyp | | | % Turnover | Std | 20 pm cyp | | | % Turnover | |
|---|---|---|---|---|---|---|---|---|---|---|
| colchicine | [col], nM | Mean | Std dev | Mean | Error | [col], nM | Mean | Std dev | Mean | Std Error |
| control | 553 | | | | | 553 | | | | |
| control | 519 | 531 | 19 | | | 519 | 531 | 19 | | |
| control | 522 | | | | | 522 | | | | |
| 1A2 | 531 | 358 | 308 | | | 520 | 522 | 8 | | |
| 1A2 | 540 | | | | | 515 | | | | |
| 1A2 | 2.94 | | | −0.80 | 0.86 | 530 | | | 1.9 | 0.8 |
| 2A6 | 507 | 500 | 11 | | | 500 | 494 | 13 | | |
| 2A6 | 506 | | | | | 479 | | | | |
| 2A6 | 488 | | | 5.9 | 1.2 | 502 | | | 7.1 | 1.4 |
| 2B6 | 526 | 528 | 10 | | | 510 | 527 | 15 | | |
| 2B6 | 519 | | | | | 536 | | | | |
| 2B6 | 538 | | | 0.70 | 1.01 | 534 | | | 0.91 | 1.56 |
| 2C8 | 481 | 489 | 8 | | | 485 | 484 | 7 | | |
| 2C8 | 496 | | | | | 490 | | | | |
| 2C8 | 490 | | | 7.9 | 0.8 | 477 | | | 9.0 | 0.7 |
| 2C9 | 505 | 505 | 11 | | | 525 | 512 | 14 | | |
| 2C9 | 494 | | | | | 497 | | | | |
| 2C9 | 515 | | | 5.1 | 1.1 | 515 | | | 3.6 | 1.5 |
| 2C19 | 539 | 520 | 19 | | | 505 | 505 | 7 | | |
| 2C19 | 519 | | | | | 498 | | | | |
| 2C19 | 502 | | | 2.2 | 2.0 | 512 | | | 5.0 | 0.8 |
| 2D6 | 542 | 535 | 7 | | | 500 | 511 | 12 | | |
| 2D6 | 528 | | | | | 523 | | | | |
| 2D6 | 535 | | | −0.65 | 0.81 | 509 | | | 4.0 | 1.3 |
| 2E1 | 538 | 534 | 4 | | | 538 | 527 | 18 | | |
| 2E1 | 530 | | | | | 537 | | | | |
| 2E1 | 533 | | | −0.41 | 0.45 | 506 | | | 1.4 | 1.8 |
| 3A4 | 538 | 529 | 12 | | | 441 | 438 | 8 | | |
| 3A4 | 515 | | | | | 443 | | | | |
| 3A4 | 533 | | | 0.52 | 1.29 | 429 | | | 17.7 | 0.8 |

Based on these experiments, several CYP450s appeared to metabolize colchicine at 43 nM including CYP3A4, CYP2B6, CYP2C9, and CYP2C19, which showed percent turnovers of 20.3, 13.3, 12.9, and 9.1% at 20 pmol CYP450, and 0.19, 1.7, 7.9, and 0.37% at 5 pmol CYP450, respectively. However, none of the enzymes showed a statistically significant level of depletion of colchicine relative to the control (p≦0.05 using a t-test) in the experiments using only 5 pmol of the CYP isozyme, although the level of colchicine measured after exposure to CYP2C9 was almost significantly different than the control (p=0.051). At 20 pmol CYP, CYP2C9, CYP2C19, and CYP3A4 showed reduction in colchicine that was significantly different from the initial colchicine present in the control (p≦0.05).

At 430 nM concentrations of colchicine CYP3A4, CYP2C8, CYP2A6, CYP2C9, and CYP2C19 were most effective with percent turnovers of 17.7, 9.0, 7.1, 3.6, and 5.0% at 20 pmol CYP450, and 0.52, 7.9, 5.9, 5.1, and 2.2% at 5 pmol CYP450, respectively. CYP2D6 also moderately metabolized 430 nM colchicine (maximum turnover of 3.96%). However, when the amount of colchicine detected in the metabolism samples were compared with the amount of colchicine detected in the control using a t-test to determine if the two were statistically different, only CYP2C8 had a p≦0.05 at both 5 and 20 pmol CYP. CYP2A6 and CYP3A4 showed statistically significant colchicine loss only at 20 pmol CYP.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of determining risk of an adverse event in administration of colchicine comprising
determining for a patient to whom colchicine is going to be administered or is being administered whether a substance that is currently being or will be administered to the patient is a substrate of CYP1A2; and
determining risk for the patient of an adverse event during coadministration of colchicine and the substance resulting from reduced metabolism of the substance by CYP1A2.

2. The method of claim 1, comprising
determining risk for the patient of an adverse event during coadministration of colchicine and the substance resulting from reduced metabolism of the substance by CYP1A2,
wherein the reduced metabolism of the substance by CYP1A2 is due to down-regulation of CYP1A2 expression by colchicine.

3. The method of claim 1, wherein determining risk comprises
accessing a pharmacy management system.

4. The method of claim 1, further comprising
administering colchicine to the patient with the substance if there is not a risk of an adverse event.

5. The method of claim 1, further comprising
administering colchicine to the patient with the substance if risk of an adverse event is determined to be acceptable.

6. The method of claim 1, further comprising
administering colchicine to the patient but not administering the substance if there is a risk of an adverse event.

7. The method of claim 1, comprising
administering colchicine to the patient but not administering the substance if there is an unacceptable risk of an adverse event.

8. The method of claim 1, wherein the patient has gout or an attack of acute gouty arthritis.

9.-10. (canceled)

11. A method of coadministration of colchicine and a substrate of CYP1A2 to a patient comprising,
administering colchicine and a substrate of CYP1A2 to a patient in need of colchicine and the substrate;
monitoring the patient during coadministration of the colchicine and the substrate; and
adjusting the dosing of colchicine or the substrate in response to the monitoring such that an adverse event associated with the coadministration of colchicine and a substrate of CYP1A2 is avoided.

12. The method of claim 11, wherein monitoring the patient comprises
monitoring the patient's plasma concentration of the substrate;
monitoring the patient for an adverse reaction associated with elevated substrate plasma concentration;
monitoring the patient for a symptom of an active agent interaction between the substrate and colchicine;
monitoring the patient for an adverse reaction resulting from coadministration of the substance and the substrate;
monitoring the patient for an adverse reaction or side effect associated with the substrate;
monitoring the patient for a substrate-associated toxicity; or
monitoring the patient for a symptom of elevated plasma concentration of the substrate.

13. The method of claim 11, wherein the patient has acute gouty arthritis; chronic gout; a cystic disease comprising polycystic kidney disease or cystic fibrosis; a lentiviral infection; a demyelinating disease of central or peripheral origin; multiple sclerosis; cancer; an inflammatory disorder comprising rheumatoid arthritis; glaucoma; Dupuytren's contracture; idiopathic pulmonary fibrosis; primary amyloidosis; recurrent pericarditis; acute pericarditis; asthma; postpericardiotomy syndrome; proliferative vitreoretinopathy; Behçet's disease; Familial Mediterranean fever; idiopathic thrombocytopenic purpura; primary biliary cirrhosis; or pyoderma gangrenosum; or is in need of enhanced bone mineral density.

14.-32. (canceled)

33. A method of administering colchicine to a patient in need thereof, comprising
receiving information that colchicine
a) is metabolized by cytochrome P450 2A6, 2B6, 2C8, 2C9 or 2C19;
b) inhibited cytochrome P450 2A6 or 2C8 enzyme activity in an in vitro inhibition study;
c) activated CYP3A4 enzyme activity in an in vitro inhibition study;
d) suppressed enzyme activity of cytochrome P450 1A2, 2A6, 2C19, 2D6, or 2E1 in an in vitro induction study; or
e) suppressed mRNA expression of cytochrome P450 1A2 in an in vitro induction study;
and
adjusting administration of colchicine and an active agent to a patient in response to the information to avoid an adverse event in the patient.

34. The method of claim 33, wherein the method further comprises
informing the patient or the patient's medical care worker that administration of colchicine with an active agent that is a known substrate of cytochrome P450 1A2, 2A6, 2C19, 2D6, or 2E1 can result in reduced metabolism of the active agent or increased plasma concentration of the active agent; or
monitoring the patient during administration of colchicine.

35. The method of claim 34, wherein monitoring the patient comprises:
monitoring the patient's plasma concentration of the active agent or colchicine;
monitoring the patient for symptoms of an active agent interaction between the active agent and colchicine;
monitoring the patient for an adverse event associated with elevated plasma concentration of the active agent; or
monitoring the patient for an adverse reaction or side effect resulting from coadministration of the active agent and colchicine.

36.-45. (canceled)
